(19) United States
(12) Patent Application Publication
(10) Pub. No.: US 2016/0271513 A1
(43) Pub. Date: Sep. 22, 2016

(54) DRYING TECHNIQUES FOR MICROFLUIDIC AND OTHER SYSTEMS

(71) Applicant: PRESIDENT AND FELLOWS OF HARVARD COLLEGE, Cambridge, MA (US)

(72) Inventors: David A. Weitz, Bolton, MA (US); Esther Amstad, Cambridge, MA (US); Frans Spaepen, Cambridge, MA (US)

(21) Appl. No.: 15/032,920

(22) PCT Filed: Oct. 29, 2014
(86) PCT No.: PCT/US14/62785

§ 371 Date: Apr. 28, 2016

Related U.S. Application Data
(60) Provisional application No. 61/897,144, filed on Oct. 29, 2013.

(51) Int. Cl. B01D 1/18; C01F 11/46; B01L 3/00; A61K 9/16; A61K 31/216; A61K 31/58; C01F 11/18; C01G 49/02

(52) U.S. Cl. CPC B01D 1/18; C01F 11/181; C01F 11/462; C01G 49/02; A61K 9/16; A61K 31/216; A61K 9/1641; A61K 31/58; B01L 3/502761; B01L 2300/0896; B01L 2200/0647

(57) ABSTRACT

The present invention generally relates to microfluidics, and to spray drying and other drying techniques. Various embodiments of the invention are generally directed to systems and methods for drying fluids contained within a channel such as a microfluidic channel. For example, a fluid may be partially or completely dried within a microfluidic channel, prior to being sprayed into a collection region. In some embodiments, the fluids may be dried relatively rapidly, resulting in spray-dried particles that are partially or completely amorphous. For instance, the fluid may contain salts, drugs, small molecules, ceramics, inorganic species, metals, sugars, polymers, etc., which may be dried to form partially or completely amorphous nanoparticles containing these species.

### RELATED APPLICATIONS

This application claims the benefit of U.S. Provisional Patent Application Ser. No. 61/897,144, filed Oct. 29, 2013, entitled “Drying Techniques for Microfluidic and Other Systems,” by Weitz, et al. incorporated herein by reference in its entirety.

### FIELD OF INVENTION

The present invention generally relates to microfluidics, and to spray drying and other drying techniques.

### BACKGROUND

Spray drying is a technique that is commonly used to dry fluids, and is often used in diverse applications such as the spray drying of food (e.g., milk powder, coffee, tea, eggs, cereal, spices, flavorings, etc.), pharmaceutical compounds (e.g., antibiotics, medical ingredients, drugs, additives, etc.), industrial compounds (e.g., paint pigments, ceramic materials, catalysts, etc.), or the like. In spray drying, a fluid to be dried is typically expelled from a nozzle into a region that is dried and/or heated in order to cause the drying of the fluid to occur. The fluid is often liquid, although other fluids or materials may also be dried, for example wet or slushy solid materials. The region used for drying may contain air, nitrogen, or other inert gases, and in some cases is heated. The fluid is typically broken up, e.g., using a nozzle, to increase the surface area and decrease the drying time of the fluid. However, many techniques offer limited control over droplet size; this limits the degree of control over the size of subsequent particles. In addition, the use of heated air may create the risk of thermal degradation of the spray-dried product in some cases.

### SUMMARY

The present invention generally relates to microfluidics, and to spray drying and other drying techniques. The subject matter of the present invention involves, in some cases, interrelated products, alternative solutions to a particular problem, and/or a plurality of different uses of one or more systems and/or articles.

In one aspect, the present invention is generally directed to a composition. In one set of embodiments, the composition comprises a plurality of particles that are substantially amorphous. In some cases, at least about 90% of the particles comprise at least about 75 wt % of a metal.

In another aspect, the present invention is generally directed to a method. According to a first set of embodiments, the method comprises acts of providing a fluidic droplet having an average diameter of less than about 100 nm, and drying the fluidic droplet within a microfluidic channel to remove at least about 30 wt % of the fluid from the droplet to produce a substantially amorphous particle comprising the species. In some cases, the droplet initially comprises less than about 10 wt % of a species contained within a fluid. In certain embodiments, the species has a solubility of at least about 0.1 g/L in the fluid.

The method in another set of embodiments, is generally directed to a method of evaporating a liquid. In certain embodiments, the method includes an act of passing a liquid droplet comprising a metal through a microfluidic channel such that at least about 20 vol % of the liquid evaporates from the droplet while the droplet is contained within the microfluidic channel to produce a substantially amorphous particle comprising at least 75 wt % of the metal. The method, in yet another set of embodiments, includes an act of passing a liquid droplet comprising a carbohydrate through a microfluidic channel such that at least about 20 vol % of the liquid evaporates from the droplet while the droplet is contained within the microfluidic channel to produce a substantially amorphous particle comprising at least 50 wt % of the carbohydrate. In still another set of embodiments, the method includes an act of passing a liquid droplet comprising a polymer through a microfluidic channel such that at least about 20 vol % of the liquid evaporates from the droplet while the droplet is contained within the microfluidic channel to produce a substantially amorphous particle comprising at least 50 wt % of the polymer.

In accordance with yet another set of embodiments, the method includes acts of providing a fluidic droplet having an average diameter of less than 100 nm, and drying the fluidic droplet within a microfluidic channel to remove at least about 50 wt % of the fluid from the droplet to produce a substantially amorphous particle. In some cases, the droplet comprises less than about 10 wt % of a species contained within a fluid.

In one set of embodiments, the method comprises passing a liquid comprising a metal through a microfluidic channel such that at least about 25 vol % of the liquid evaporates within the microfluidic channel, and spraying the unevaporated liquid into a collection region external of the microfluidic channel to produce amorphous particles comprising at least 75 wt % of the metal. In another set of embodiments, the method comprises passing a liquid comprising a carbohydrate through a microfluidic channel such that at least about 25 vol % of the liquid evaporates within the microfluidic channel, and spraying the unevaporated liquid into a collection region external of the microfluidic channel to produce amorphous particles comprising at least 50 wt % of the carbohydrate. In yet another set of embodiments, the method comprises passing a liquid comprising a polymer through a microfluidic channel such that at least about 25 vol % of the liquid evaporates within the microfluidic channel, and spraying the unevaporated liquid into a collection region external of the microfluidic channel to produce amorphous particles comprising at least 50 wt % of the polymer.

In another aspect, the present invention encompasses methods of making one or more of the embodiments described herein, for example, spray drying and other drying techniques involving microfluidics. In still another aspect, the present invention encompasses methods of using one or more of the embodiments described herein, for example, spray drying and other drying techniques involving microfluidics.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 illustrates a channel used for drying a fluid, in accordance with one embodiment of the invention;

FIGS. 2A-2C illustrate a microfluidic device for drying a fluid, in accordance with another embodiment of the invention;

FIGS. 3A-3I illustrate a microfluidic device for drying a fluid, in accordance with one embodiment of the invention;

FIGS. 4A-4E illustrate the morphology of certain spray-dried particles, in accordance with certain embodiments of the invention;

FIGS. 5A-5F illustrate nucleation and crystal growth, in some embodiments of the invention;

FIGS. 6A-6I illustrate certain inorganic nanoparticles, in some embodiments of the invention;

FIGS. 7A-7H illustrate certain characteristics of a microfluidic device, in yet another embodiment of the invention;

FIGS. 8A-8H illustrate operation of a spray dryer in still another embodiment of the invention;

FIGS. 9A-9D illustrate flow profiles in yet another embodiment of the invention;

FIGS. 10A-10F illustrate spray-dried CaCO₃ particles of some embodiments of the invention; and

FIGS. 11A-11F illustrate spray-dried organic solutions of certain embodiments of the invention;

FIGS. 12A-12D illustrate the stability of amorphous fenofibrate, in one embodiment of the invention;

FIGS. 13A-13C illustrate the spray drying of drugs with a T_{g} above room temperature, in certain embodiments of the invention;

FIGS. 14A-14D illustrate co-spray drying fenofibrate with Pluronics excipients, in accordance with some embodiments of the invention;

FIGS. 15A-15D illustrate co-spray drying danazol with Pluronic excipients, in some embodiments of the invention;

FIGS. 16A-16B illustrate co-spray drying drugs with poly(vinyl pyrrolidone), in yet other embodiments of the invention;

FIGS. 17A-17B illustrate the spray drying of drugs onto a PVP matrix, in still another embodiment of the invention.

### DETAILED DESCRIPTION

The present invention generally relates to microfluidics, and to spray drying and other drying techniques. Various embodiments of the invention are generally directed to systems and methods for drying fluids contained within a channel such as a microfluidic channel. For example, a fluid may be partially or completely dried within a microfluidic channel, prior to being sprayed into a collection region. In some embodiments, the fluids may be dried relatively rapidly, resulting in spray-dried particles that are partially or completely amorphous. For instance, the fluid may contain salts, drugs, small molecules, ceramics, inorganic species, metals, sugars, polymers, etc., which may be dried to form partially or completely amorphous nanoparticles containing these species.

Certain aspects of the invention are generally directed to systems and methods for forming nanoparticles. As discussed, in some cases, the fluid is dried relatively quickly, such that species contained within the fluid (e.g., dissolved and/or suspended therein) do not have time to crystallize as the fluid dries, and thus, the species form amorphous solids instead, or at least regions of the solid may be amorphous. Thus, for example, a plurality of fluidic droplets, containing a species, may be dried to form particles comprising the species, which may be partially or completely amorphous. In some cases, the particles are nanoparticles.

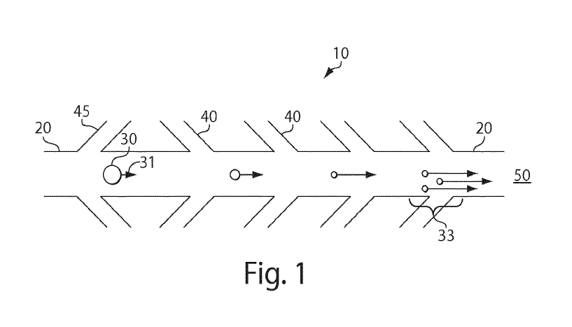

In one set of embodiments, the fluid is dried by forming fluidic droplets and causing drying of the droplets within a channel such as a microfluidic channel, such as described below and in FIG. 1. However, it should be understood that the invention is not limited to only drying within a specific type or configuration of channel or microfluidic channel. In other embodiments of the invention, fluidic droplets are dried relatively quickly in other configurations of microfluidic channels to produce particles that are partially or completely amorphous. For example, in one set of embodiments, fluidic droplets (such as those described herein) are dried within a microfluidic channel to remove at least about 50 wt % of the fluid from the droplet to produce a substantially amorphous particle. In some embodiments, the fluid may be dried using other techniques instead of within a channel such as a microfluidic channel. Any technique for drying a fluid quickly can be used in some cases, as is discussed herein.

The liquid or other fluid to be dried may be present within a channel within the spray dryer in any suitable form, for example, as individual droplets (such as those previously discussed), as a film (e.g., coating a wall of the channel), a jet, or the like. If droplets are present, the droplets may exhibit dripping behavior, jetting behavior, etc. In certain instances, as discussed herein, if the fluid is present as a liquid, the liquid may at least partially evaporate within the channel. Thus, for example, the liquid (or other fluid) may be relatively volatile, e.g., having a relatively high vapor pressure or partial pressure. In addition, in some cases, the liquid or other fluid may be disrupted to form droplets, which may be partially or fully dried within the channel in certain embodiments, e.g. forming particles.

Any suitable liquid may be dried. For example, the liquid may be aqueous (e.g., miscible in water), or an oil or other non-aqueous liquid (e.g., immiscible in water). Examples of aqueous liquids include, but are not limited to, water, alcohols (e.g., butanol (e.g., n-butanol), isopropanol (IPA), propanol (e.g., n-propanol), ethanol, methanol, acetone, dimethylformamide, dimethyl sulfoxide, or the like), saline solutions, blood, acids (e.g., formic acid, acetic acid, or the like), amines (e.g., dimethyl amine, diethyl amine, or the like), mixtures of these, and/or other similar fluids. It should also be understood that although liquids are described in many of the examples and embodiments below, the present invention is not limited to only liquids and methods for drying liquids, but also encompasses the drying of other fluids or materials, for example, wet or slushy solid materials, viscoelastic solids, liquid emulsions, syrupy materials, or the like, in still other embodiments of the invention. For example, a material may contain a liquid or other volatile fluid which is to be dried.

In various embodiments, the droplets within the channel (before or after disruption), may have an average diameter of less than about 1 mm, less than about 500 micrometers, less than about 300 micrometers, less than about 200 micrometers, less than about 100 micrometers, less than about 75 micrometers, less than about 50 micrometers, less than about 30 micrometers, less than about 25 micrometers, less than about 20 micrometers, less than about 15 micrometers, less than about 10 micrometers, less than about 5 micrometers, less than about 3 micrometers, less than about 2 micrometers, less than about 1 micrometer, less than about 500 nm, less than about 300 nm, less than about 100 nm, or less than about 50 nm. The average diameter of the droplets may also be at least about 30 nm, at least about 50 nm, at least about 100 nm, at least about 300 nm, at least about 500 nm, at least about 1 micrometer, at least about 2 micrometers, at least about 3 micrometers, at least about 5 micrometers, at least about 10 micrometers, at least about 15 micrometers, or at least about 20 micrometers in certain cases. The “average diameter” of a population of droplets is the arithmetic average of the diameters of the droplets.

In certain cases, the droplets may be relatively small at the time crystallization nuclei start to form. This does not necessarily require that the droplets initially be relatively small, however. For example, the droplets can be relatively large if the initial solute concentration is low. As the fluid leaves the droplets, the droplets can shrink in size, causing the species concentration within the droplet to increase. However, crystallization nuclei may form only when the species concentration exceeds the saturation concentration. Thus, the droplet may initially shrink from relatively larger sizes prior to crystallization.

In some aspects, a fluid within a channel may contain a species such as a chemical, biochemical, or biological entity, a cell, a particle, a bead, gases, molecules, a pharmaceutical agent, a drug, DNA, RNA, proteins, a fragrance, a reactive agent, a biocide, a fungicide, a pesticide, a preservative, or the like. Thus, the species can be any substance that can be contained in a fluid and can be differentiated from the fluid containing the species. For example, the species may be dissolved or suspended in the fluid. The species may be present in one or more of the fluids. If the fluids contain droplets, the species can be present in some or all of the droplets. Additional non-limiting examples of species that may be present include, for example, biochemical species such as nucleic acids such as siRNA, RNAi and DNA, proteins, peptides, or enzymes. Still other examples of species include, but are not limited to, nanoparticles, quantum dots, fragrances, proteins, indicators, dyes, fluorescent species, chemicals, or the like. As yet another example, the species may be a drug, pharmaceutical agent, or other species that has a physiological effect when ingested or otherwise introduced into the body, e.g., to treat a disease, relieve a symptom, or the like. In some embodiments, the drug may be a small-molecule drug, e.g., having a molecular weight of less than about 2000 Da, less than about 1500 Da, less than about 1000 Da, or less than about 500 Da.

In another set of embodiments, the species may be one or more metal species, including alkali metals and alkali earth metals, as well as other metals within the Periodic Table that are not alkali metals or alkali earth metals. In some cases, the metal may be used to form amorphous particles of pure metal (i.e., as opposed to species such as NaCl or BaSO₄, where the metal is bound to another element and is not present in pure form). For example, particles that are formed may comprise at least about 50 wt %, at least about 60 wt %, at least about 70 wt %, at least about 75 wt %, at least about 80 wt %, at least about 85 wt %, at least about 90 wt %, at least about 95 wt %, or at least about 99 wt % of the metal. Non-limiting examples of such metals include beryllium, magnesium, zinc, aluminum, gallium, indium, iron, cobalt, copper, gold, silver, titanium, nickel, etc. In some cases, mixtures or alloys of any of these and/or other metals may also be used. In some cases, the metals may be present as one or more ions (e.g., Be²⁺, Mg²⁺, Zn²⁺, Al³⁺, Ga²⁺, In2+, Fe²⁺, Fe³⁺, Co²⁺, Cu⁺, Cu²⁺, Au²⁺, Au³⁺, Ag⁺, Ni²⁺, etc.) that are reduced to a metal state, e.g., during the drying process. The metals may be dissolved and/or suspended in water, or another suitable liquid (e.g., including those described herein). In some cases, the metals may initially be present within a fluidic droplet as dissolved ions, then as the droplet dries, the metal coalescences to form a solid particle, or regions within a solid particle. As discussed herein, in some cases, the drying process may be sufficiently rapid such that the solid particle comprising the metal that is formed is partially or completely amorphous.

The species, in other embodiments, may include one or more sugars or carbohydrates. Non-limiting examples include glucose, sucrose, fructose, mannose, trehalose, starch, cellulose, dextran, cyclodextrin, alginate, or the like. The sugars or carbohydrates may be unsubstituted or substituted in some cases, e.g., with OH or halogen groups (Cl, I, F, etc.). The sugars or carbohydrates may be dissolved and/or suspended in water, or another suitable liquid (e.g., including those described herein). In some cases, the sugar or carbohydrate may have a relatively low molecular weight, e.g., less than about 2 kDa, less than about 1.5 kDa, less than about 1 kDa, or less than about 500 Da in some cases. In addition, in certain cases, more than one sugar and/or carbohydrate may be used, including any of these and/or other sugars or carbohydrates. In still another set of embodiments, the species may include one or more polymers. Examples of suitable polymers include, but are not limited to, poly(ethylene glycol), poly(oxazoline), poly(acrylic acid), poly(lactic acid), poly(L-lysine), poly(lactic-co-glycolide acid), shellac, chitin, chitosan, cyclodextrin, etc. Combinations of these polymers and/or other polymers may also be used in some instances. The polymers may be dissolved and/or suspended in water, or another suitable liquid (e.g., including those described herein).

Such species may be partially or completely dissolved or suspended within the liquid used to form the fluidic droplets, and as the droplets dry, the species coalesce or precipitate to form particles. In some cases, the particles may be partially or completely amorphous, e.g., if the particles are dried relatively rapidly, such that the species do not have sufficient time to crystallize. In some cases, the particles that are formed may comprise at least about 10 wt %, at least about 20 wt %, at least about 30 wt %, at least about 40 wt %, at least about 50 wt %, at least about 60 wt %, at least about 70 wt %, at least about 75 wt %, at least about 80 wt %, at least about 85 wt %, at least about 90 wt %, at least about 95 wt %, or at least about 99 wt % of the species, e.g., sugar, carbohydrate, polymer, metal, or other species, etc.

In yet another set of embodiments, the species may be one or more inorganic species, such as salts or ceramics. Non-limiting examples include, but are not limited to, CaCO₃, NaCl, BaSO₄, FeO, Fe₂O₃, Fe₃O₄, or the like. Typically, an inorganic compound is one that does not contain any C—H covalent bonds, although in some cases, the inorganic compound may contain carbon atoms, such as CaCO₃, and/or hydrogen atoms, such as HCl, Ca(HCO₃)₂, or H₂CO₃. Examples of inorganic species include, but are not limited to, those discussed in International Patent Application No. PCT/US2011/048822, filed Aug. 23, 2011, entitled “Particles for Drug Delivery and Other Applications,” published as WO 2012/027378 on Mar. 1, 2012, incorporated herein by reference in its entirety.

As a specific non-limiting example, a first fluid containing carbonate ions and a second fluid containing calcium ions may be mixed together within a droplet, e.g., as the droplet is formed, where the carbonate ions and the calcium ions combine to form CaCO₃, which under some conditions may precipitate, e.g., as is discussed herein. Other ions may be used instead of or in addition to calcium ions, for example, magnesium ions, sodium ions, potassium ions, silicon ions, or the like. Carbonate and/or other ions may be introduced into the first fluid using any suitable technique. For instance, carbonate salts such as Na₂CO₃, K₂CO₃, or (NH₄)₂CO₃, NaHCO₃, KHCO₃, (NH₄)HCO₃, etc. may be dissolved in the first fluid, or salts such as CaCl₂ (optionally in the form of a hydrate such as CaCl₂.2H₂O), Ca(NO₃)₂, calcium acetate, MgCl₂, Mg(NO₃)₂, magnesium acetate, NaCl, Na₂CO₃, NaNO₃, sodium acetate, KCl, K₂CO₃, KNO₃, potassium acetate, etc. may be dissolved in the second fluid. In some cases, the precipitate may comprise more than one carbonate (for example, one or more of calcium carbonate, magnesium carbonate, sodium carbonate, potassium carbonate, etc.).

In certain embodiments, the fluidic droplets are created by forming droplets from two separate fluids containing species that react together (e.g., by precipitation, changes in pH, chemical reaction, or the like) to produce the inorganic species. In some cases, the species may have relatively low solubility, and thus precipitate upon reaction, e.g., when the droplets are formed. Non-limiting examples of techniques useful for forming droplets from two different fluid sources include those described in U.S. patent application Ser. No. 11/246,911, filed Oct. 7, 2005, entitled “Formation and Control of Fluidic Species,” by Link, et al., published as U.S. Patent Application Publication No. 2006/0163385 on Jul. 27, 2006, and U.S. patent application Ser. No. 11/360,845, filed Feb. 23, 2006, entitled “Electronic Control of Fluidic Species,” by Link, et al., published as U.S. Patent Application Publication No. 2007/0003442 on Jan. 4, 2007, each incorporated herein by reference in its entirety. In some embodiments, the species may be formed within the combined droplet and allowed to dry relatively quickly, e.g., to form partially or completely amorphous comprising the species, before the species has time to crystallize.

However, it should be understood that the invention is not limited only to relatively insoluble species or species that can only be suspended in the fluid. Highly soluble species, such as NaCl or KCl, are also contemplated in other embodiments. Other examples of highly soluble species include, but are not limited to, CaCl₂, MgCl₂, HAuCl₄, Ag(NO₃), etc. Combinations of any of these and/or other species may also be used in some cases. In some cases, a relatively highly soluble species may be dissolved in a fluid that is used to form the droplets, without necessarily requiring any chemical reactions such as those previously described. The soluble species may have, for example, a solubility of at least about 0.1 g/L, at least about 0.2 g/L, at least about 0.3 g/L, at least about 0.5 g/L, at least about 1 g/L, at least about 2 g/L, at least about 3 g/L, at least about 5 g/L, at least about 10 g/L, at least about 20 g/L, at least about 30 g/L, at least about 50 g/L, at least about 100 g/L, at least about 150 g/L, at least about 200 g/L, at least about 250 g/L, or at least about 300 g/L in the fluid. Such species often exhibit a strong tendency to crystallize, since the concentration of the species increases as a fluid containing the species dries, thereby resulting in a high concentration of species in the fluid just before solidification, which normally facilitates crystallization. However, surprisingly, under conditions such as those described herein, fluids containing such species may be dried to form amorphous particles, or amorphous regions within the particles, rather than crystalline particles. Accordingly, in certain embodiments of the invention, fluidic droplets having sizes such as those discussed herein may be produced containing such solubilities, and the fluidic droplets can be subsequently dried to produce partially or completely amorphous nanoparticles.

In some embodiments, the fluidic droplets that are to be dried may contain a relatively low concentration of the species. Without wishing to be bound by any theory, it is believed that solutes can start to form crystalline nuclei if their concentration exceeds the saturation concentration. In some cases, a lower the initial solute concentration can reduce the time crystalline nuclei can form as the solute concentration exceeds its saturation concentration only in late stages of the drop evaporation. Thus, using lower concentrations of solute may facilitate the formation of amorphous particles, or amorphous regions within the particles. Thus, for example, the fluidic droplets may initially contain less than about 50 wt % of the species, or in some cases, the droplets may contain less than about 25 wt %, less than about 15 wt %, less than about 10 wt %, less than about 8 wt %, or less than about 5 wt % of the species. For instance, in one set of embodiments, a fluidic droplet having an average diameter of less than about 100 nm or less than about 50 nm, where the droplet comprises less than about 10 wt % solute contained within a fluid may be dried to produce a substantially amorphous particle. As discussed, the species may be a metal, a sugar, a carbohydrate, a polymer, a salt, an inorganic species, or any other suitable species as is discussed herein.

It should also be understood that the droplet may also contain more than one species, including more than one of any of the species, in any configuration or combination, described herein. For example, the droplets may contain more than one salt, more than one sugar, a metal and a polymer, a salt and a metal, a salt and a polymer, or the like. In addition, in certain embodiments, relatively small fluidic droplets are used, and/or the droplets are elongated or disrupted to produce smaller droplets, as is discussed herein. Without wishing to be bound by any theory, it is believed that smaller droplets facilitates more rapid drying, e.g., due to the increased surface-to-volume ratio of the droplets, and/or lesser amounts of fluid that would need to be removed from the droplets in order to effectuate drying.

Thus, for example, the average diameter of the droplets within the a channel may be less than about 1 mm, less than about 500 micrometers, less than about 300 micrometers, less than about 200 micrometers, less than about 100 micrometers, less than about 75 micrometers, less than about 50 micrometers, less than about 30 micrometers, less than about 25 micrometers, less than about 20 micrometers, less than about 15 micrometers, less than about 10 micrometers, less than about 5 micrometers, less than about 3 micrometers, less than about 2 micrometers, less than about 1 micrometer or less in certain cases. For example, droplets having such sizes may be created within a channel, such as a microfluidic channel as is discussed herein. As mentioned, the species may be partially or completely dissolved and/or suspended within the fluid used to form the fluidic droplets, and as the droplets dry, the species precipitate to form particles. The particles may be partially or completely amorphous in some cases. For instance, if the particles are dried relatively rapidly, the species may not have sufficient time to crystallize. In some embodiments, the particles that are formed may comprise at least about 50 wt %, at least about 60 wt %, at least about 70 wt %, at least about 75 wt %, at least about 80 wt %, at least about 85 wt %, at least about 90 wt %, at least about 95 wt %, or at least about 99 wt % of the species, e.g., an inorganic species, metal, polymer, salt, etc., as previously discussed.

For example, in one set of embodiments, relatively small fluidic droplets may be produced, e.g., having an average diameter of less than about 100 nm or less than about 50 nm, containing a species therein, which can then be dried to produce a substantially amorphous particle containing the species, e.g., as determined by a lack of long-range order, indicated by the absence of statistically significant diffraction peaks in a single-crystal X-ray diffraction spectrum, or by using other techniques such as differential scanning calorimetry (DSC), e.g., as described herein. Surprisingly, almost any species can be dried to produce amorphous particles, including metals, polymers, carbohdyrates, inorganic species, ionic salts, etc., including ionic salts that are highly soluble and/or normally readily crystallize under typical ambient conditions. In some cases, amorphous particles may be produced starting with fluidic droplets having a relatively low concentration of species initially present, e.g., less than about 20 wt %, less than about 10 wt %, or other lower weight percentages as described herein. As discussed, a lower concentration of species may minimize the time crystalline nuclei can form during the drying process of such droplets, thereby minimizing the probably crystalline nucleic can form. This can lead to the formation of amorphous particles, or at least amorphous regions within the particle.

As mentioned, in some cases, at least a portion of the fluids within the individual droplets may harden or solidify, e.g., within the collection region and/or within a microfluidic channel. For example, some of the droplets, and/or a portion of some of the droplets, can harden to form particles. In addition, in some cases, the particles may form or solidify after the drops exit the device. The particles can then be subsequently collected. The particles may, in some embodiments, be smaller than the fluidic droplets. The size of the particles can be determined, for instance, by the initial solute concentration and the drop size. In some embodiments, the particles are monodisperse, e.g., as discussed above, and/or the particles may be spherical, or non-spherical in certain cases. In some cases, some or all of the particles may be microparticles and/or nanoparticles. Microparticles generally have an average diameter of less than about 1 mm (e.g., such that the average diameter of the particles is typically measured in micrometers), while nanoparticles generally have an average diameter of less than about 1 micrometer (e.g., such that the average diameter of the particles is typically measured in nanometers). In some cases, the nanoparticles may have an average diameter of less than about 100 nm. In some cases, the particles may have a distribution in diameters such that at least about 50%, at least about 60%, at least about 70%, about 80%, at least about 85%, at least about 90%, at least about 95%, at least about 97%, or at least about 99% of the droplets have a diameter that is no more than about 10% different, no more than about 7% different, no more than about 5% different, no more than about 4% different, no more than about 3% different, no more than about 2% different, or no more than about 1% different from the average diameter of the particles. In addition, as discussed, the particles may be partially or completely amorphous in some cases.

In one set of embodiments, the average diameter of the particles is less than about 1 mm, less than about 500 micrometers, less than about 300 micrometers, less than about 200 micrometers, less than about 100 micrometers, less than about 75 micrometers, less than about 50 micrometers, less than about 30 micrometers, less than about 25 micrometers, less than about 20 micrometers, less than about 15 micrometers, less than about 10 micrometers, less than about 5 micrometers, less than about 3 micrometers, less than about 2 micrometers, less than about 1 micrometer, less than about 500 nm, less than about 300 nm, less than about 100 nm, or less than about 50 nm. The average diameter of the particles may also be at least about 30 nm, at least about 50 nm, at least about 100 nm, at least about 300 nm, at least about 500 nm, at least about 1 micrometer, at least about 2 micrometers, at least about 3 micrometers, at least about 5 micrometers, at least about 10 micrometers, at least about 15 micrometers, or at least about 20 micrometers in certain cases.

In addition, various embodiments of the present invention are generally directed to systems and methods for at least partially drying a liquid droplet (or other fluidic droplet) within a channel such as a microfluidic channel, for example, such that at least about 10 vol % of the liquid within the droplet evaporates while the droplet is contained within the channel, prior to exiting the microfluidic channel, e.g., exiting through a nozzle into a collection region. In some embodiments, even higher amounts of drying may occur within the channel, e.g., at least about 20 vol %, at least about 30 vol %, at least about 40 vol %, at least about 50 vol %, at least about 60 vol %, at least about 70 vol %, at least about 75 vol %, at least about 80 vol %, at least about 85 vol %, at least about 90 vol %, or at least about 95 vol % of the liquid may evaporate from the droplet while the droplet is contained within the channel. As mentioned, in some embodiments, the droplets may solidify, e.g., to form particles, as liquid evaporates therefrom. For instance, a species contained within the droplets may remain to form particles as liquid evaporates. In some cases, a substantial portion of the particles may be formed from the species. The particles may form within the microfluidic channel, and/or upon expulsion of the liquid droplets into the collection region. The solid particles may be crystalline, or amorphous in certain embodiments, for example, depending on the amount of time crystalline nuclei can form as the fluid within the droplets evaporates. Typically the droplets form particles as the concentration of the species reaches or exceeds the saturation limit, although in some cases, the concentration may substantially exceed the saturation limit, e.g., such that supersaturation occurs.

In some cases, as mentioned, the drying time may be relatively rapid, e.g., such that the species within the fluidic droplet does not have sufficient time to crystallize as the fluidic particle dries and fluid is removed from the droplet, and thus, the species forms a solid phase that is partially or completely amorphous. For example, the drying time of a fluidic droplet may be less than about 50 microseconds, less than about 25 microseconds, less than about 20 microseconds, less than about 15 microseconds, less than about 10 microseconds, less than about 5 microseconds, less than about 3 microseconds, less than about 1 microseconds, or less in some cases.

The drying time within a channel, such as a microfluidic channel as is discussed herein, may be controlled, for instance, by controlling the size of the fluidic droplets contained within the microfluidic channel, by controlling the concentration of the species within the fluidic droplet, by controlling characteristics of the gases within the microfluidic channel (e.g., the temperature, relative humidity, pressure, flow rate, number of channels for inserting gas, angle of the channels, etc., as is discussed in detail herein), etc. In some embodiments, at least about 20 vol %, at least about 30 vol %, at least about 40 vol %, at least about 50 vol %, at least about 60 vol %, at least about 70 vol %, at least about 75 vol %, at least about 80 vol %, at least about 85 vol %, at least about 90 vol %, or at least about 95 vol % of the liquid may evaporate from the droplet while the droplet is contained within the channel. In some cases, controlling the concentration of species within the fluidic droplet may be used to control the time crystalline nuclei can form.

The degree of crystallization (or lack thereof) within particles produced as discussed herein may be determined using techniques known to those of ordinary skill in the art, such as X-ray diffraction (XRD) measurements or differential scanning calorimetry (DSC) techniques. For example, in one set of embodiments, a sample is analyzed using DSC to determine if the sample shows any melting peaks (Tₘ) that would be indicative of crystallinity in the sample; an amorphous sample would not contain any melting peaks, although other peaks, such as glass transition temperature changes (T_{g}), may be present. Tₘ peaks can be readily identified using a suitable control sample that is known to be crystalline. As another example, amorphous particles may be determined by as a lack of statistically significant diffraction peaks in a single-crystal X-ray diffraction spectrum produced using commonly-accepted X-ray diffraction measurements. The X-ray source typically used to perform these measurements is a CuKα (alpha) source with an X-ray wavelength of 0.15418 nm.

Certain embodiments of the present invention are generally directed to spray dryers for at least partially drying fluids (typically, liquids), e.g., to produce particles such as microparticles or nanoparticles. Other examples of spray dryers are discussed in U.S. Provisional Patent Application Ser. No. 61/704,422, filed Sep. 21, 2012, entitled “Systems and Methods for Spray Drying in Microfluidic and Other Systems,” incorporated herein by reference in its entirety. In one set of embodiments, the fluid may contain one or more species, as previously discussed, which may be dried to form nanoparticles, e.g., that are partially or completely amorphous. In a spray dryer, a fluid is dried, at least in part, by spraying the fluid as small droplets, e.g., through a nozzle into a collection region. However, in some embodiments, as discussed herein, the fluid may be at least partially dried prior to being sprayed into the collection region. For example, gases such as air may be directed into a microfluidic channel containing a fluid (which may be present within the channel, e.g., as droplets or films), which can cause at least partial drying of the fluid within the channel and/or cause the liquid to become disrupted to form smaller droplets, which may enhance drying.

In some embodiments, a fluid may be accelerated within the channel due to the introduction of such gases. In some cases, fluids within the channel may become elongated or disrupted under certain conditions, e.g., breaking into smaller droplets. This may speed up or accelerate the drying process. In addition, in certain embodiments, evaporation may occur within the channel more quickly, such that the air within the channel does not have to be heated. Furthermore, in some instances, the fluids within the channel may reach supersonic speeds, further increasing the rate of evaporation. Thus, for instance, the droplets may partially or completely dry within the channel, e.g., forming particles, and/or the droplets may be expelled into a drying region (for example, a region that is heated and/or has reduced humidity) to finish the drying process, e.g., in the manner of a conventional spray dryer.

Spray drying techniques such as those discussed herein may be used in a variety of applications where drying is desired. For example, spray drying may be used to dry thermally sensitive materials or thermally degradable materials, and/or to dry a fluid. In some cases, spray drying may also be used to create relatively uniform particles, e.g., due to drying of the fluid at a controlled rate. In some cases, the fluid may comprise one or more solvents, e.g., a mixture of solvents. Also, as discussed herein, in some cases, spray drying may be used to create nanoparticles that are partially or completely amorphous.

One example of an embodiment of the invention is now described with respect to FIG. 1, although other configurations may be used in other embodiments, e.g., as discussed in more detail below. In FIG. 1, microfluidic includes a microfluidic channel 20 in which fluidic droplet 30 can flow prior to being expelled from a nozzle into collection region 50, which may be heated and/or contain relatively low humidites in some cases. The microfluidic system may be formed from any suitable materials, for example, a polymer such as polydimethylsiloxane. Microfluidic channel 20 is straight in this figure, although microfluidic channel 20 need not be in other embodiments. Microfluidic channel 20 also may have a constant or a varying cross-sectional area, e.g., one that increases or decreases downstream. In addition, although only one fluidic droplet 30 is discussed here for purposes of clarity, in other embodiments, more than one fluidic droplet may be present within microfluidic channel 20.

In certain embodiments, while fluidic droplet 30 flows through microfluidic channel 20, at least some liquid from fluidic droplet 30 may evaporate. For example, if fluidic droplet 30 comprises a liquid carrying a species (e.g., suspended or dissolved therein), at least some of the liquid may evaporate from the droplet, and in certain embodiments, sufficient liquid may evaporate such that the droplet is able to solidify, e.g., to form a particle containing or even consisting essentially of the species therein. In addition, in some cases, fluidic droplet 30 may flow at relatively high velocities, which may facilitate drying and evaporation of liquid from the droplet. In contrast, in many other spray-drying systems, most of the drying occurs after the fluidic droplets have been expelled from the nozzle into a drying region. In addition, in certain embodiments of the present invention, the droplet may not necessarily solidify, and still remain at least partially liquid or fluid. Furthermore, in certain embodiments, the droplet may dry to the point of supersaturation without necessarily solidifying into a particle.

In one set of embodiments, the evaporation process may be facilitated by heating microfluidic channel 20, and/or by exposing fluidic droplet 30 to a gas such as air, into which the evaporating liquid is able to evaporate into. The gas may be heated and/or dried in some cases. However, in some embodiments, the gas may not be heated; this may be useful, for example, in the drying of thermo-sensitive materials. The gas may be present in microfluidic channel 20 when fluidic droplet 30 is introduced therein, and/or the gas may be introduced into microfluidic channel 20 at one or more locations while fluidic droplet 30 flows within the channel. For instance, as is shown in FIG. 1, a plurality of side channels 40 intersect microfluidic channel 20. Side channels 40 may each intersect microfluidic channel 20 at any suitable angle (e.g., a right angle, or a non-right angle such as an acute angle, an obtuse angle, etc.), and the various side channels may each intersect at the same or different angles. For example, as is shown here, side channels 40 are positioned at about 45° (relative to the upstream direction) to allow the entering gas to assist the flow of fluidic droplet 30 within the channel. In some embodiments, the entering gas may also cause fluidic droplet 30 to accelerate within microfluidic channel 20 (as depicted by arrows 31 of increasing length within the channel), and under some conditions, such that fluidic droplet 30 is sheared or disrupted into smaller fluidic droplets, as are illustrated by droplets 33 in FIG. 1.

Also shown in this figure are optional side channels 45, which intersect microfluidic channel 20 upstream of side channels 40. In this example, side channels 45 intersect channel 20 at an angle of about 135°, although other angles (acute, right, or obtuse) are possible in other embodiments. Side channels 45, when present, may be used to introduce a gas into microfluidic channel 20 to cause a fluid entering microfluidic channel 20 to begin forming fluidic droplets 30, e.g., in the manner of a flow-focusing device. As a non-limiting example, side channels 45 may be positioned so as to cause the flow of droplets within microfluidic channel 20 to move more rapidly, where the droplets break up to form smaller fluidic droplets 30 at essentially the same position within the channel. In some cases, the droplets are broken into smaller droplets by the application of high shear forces on the droplets.

The above discussion is a non-limiting example of an embodiment of the present invention that can be used to dry a fluid. However, other embodiments are also possible. For instance, some aspects of the invention are directed to systems and methods of drying or otherwise manipulating fluids in a channel such as a microfluidic channel. In certain embodiments, for example, the present invention is generally directed to a spray dryer for use in drying liquids or other fluids or materials, e.g., to produce particles or solids, or at least to promote drying. In some embodiments, the spray dryer contains an article containing one or more channels such as microfluidic channels, through which a liquid or other fluid is at least partially dried therein.

A variety of methods can be used to accelerate a fluid within a channel (e.g., present as droplets, a film, etc.), or otherwise change its velocity, in addition to the introduction of air and/or other gases into the channel, e.g., through one or more side channels as noted herein. As non-limiting examples, a second liquid or fluid may be used to accelerate the fluid, an external force may be applied to the fluid (e.g., gravitational, centripetal, etc.), or if the fluid is magnetically or electrically susceptible, the application of suitable magnetic or electric fields, respectively, may be used to accelerate the fluid within the channel, e.g., at one or more accelerator regions, which may be the same or different. Thus, as a non-limiting example, a liquid (e.g., a droplet, or a film of liquid) within a channel may be accelerated at a first accelerator region through introduction of a gas or other fluid, and accelerated at a second accelerator region through introduction of a gas or other fluid (which may be the same or different from the first accelerator region), an electric field, a magnetic field, gravity, or the like. There may be any suitable number of accelerator regions present within the device, e.g., 2, 3, 4, 5, 6, 7, 8, etc., and the acceleration techniques that are used may be the same or different.

The article can be formed, in accordance with one set of embodiments, from polymeric, flexible, and/or elastomeric polymers and/or other materials, e.g., silicone polymers such as polydimethylsiloxane (“PDMS”), glass, thermoplastics, metals, etc. In some embodiments, the article may comprise or even consist essentially of such polymers and/or other materials. Other examples of potentially suitable polymers and other materials are discussed in detail below. The article may be planar, or non-planar in some embodiments (e.g., curved). The article can be formed from a material that is at least partially mechanically deformable in some cases, e.g., such that the article can be visibly mechanically deformed by an average person without the use of tools. In other embodiments, however, the article may be formed of more relatively rigid materials such that the article is not as mechanically deformable by the average person.

As mentioned, in one set of embodiments, the channel through which a liquid or other fluid can flow may be intersected by one or more side channels. Any suitable number of side channels may be present, e.g., 1, 2, 3, 4, 5, 6, 7, 8, 9, 10, 11, 12, etc. The side channels can intersect the main channel at any suitable angle (e.g., a right angle, an acute angle, an obtuse angle, etc.), and the side channels can each intersect the main channel at the same or different angles. For example, the angle of intersection may be about 20°, about 30°, about 40°, about 45°, about 50°, about 60°, about 70°, about 80°, about 90°, about 100°, about 110°, about 120°, about 130°, about 135°, about 140°, about 150°, or about 160°. The side channels may be positioned or angled, for instance, such that gases entering the main channel from the side channels cause acceleration and/or drying of the liquid or other fluid. Thus, for example, if a plurality of side channels are present, the liquid or other fluid may be accelerated within the channel at one or more locations within the channel, e.g., due to gases entering from one or more of the side channels.

In one set of embodiments, one or more of the side channels are positioned at an acute angle relative to the main channel, which may facilitate the entry of gases into the main channel, e.g., such that the gases flow downstream in the main channel, which may be used to increase the velocity of liquids or other fluids contained within the main channel. Non-limiting examples of such side channels may be seen in FIG. 1 with side channels 40 intersecting main channel 20. In certain cases, more than one such side channel can be used. For instance, in some cases, the side channels may be positioned in pairs on either side of the main channel. This may be useful, for example, to keep the fluid within the main channel moving downstream without getting pushed to one side or the other. However, in other embodiments, the side channels may not necessarily intersect in pairs along the main channel.

Also shown in FIG. 1 are side channels 45. In one set of embodiments, such side channels may be positioned relative to the main channel such that these channels are arranged in a “flow-focusing” configuration, e.g., in which a first fluid in a first channel is sheathed or surrounded by a second fluid delivered using side channels (e.g., a second channel and sometimes a third channel or additional channels) in order to cause the first fluid to form discrete droplets contained within the second fluid. The first fluid and the second fluid can be miscible or immiscible. Channel configurations to create such discrete droplets may be found, for example, in U.S. patent application Ser. No. 11/024,228, filed Dec. 28, 2004, entitled “Method and Apparatus for Fluid Dispersion,” by Stone, et al., now U.S. Pat. No. 7,708,949, issued May 4, 2010, incorporated herein by reference in its entirety.

Unlike side channels 40, side channels 45 intersect main channel 20 at an obtuse angle in FIG. 1, rather than an acute angle. However, the angle of intersection may also be, in other embodiments, a right angle or an acute angle, e.g., as discussed above (or in some embodiments, no such side channels 45 may be present). Any such angle may be used, e.g., channel at the same or different angles. For example, the angle of intersection may be about 20°, about 30°, about 40°, about 45°, about 50°, about 60°, about 70°, about 80°, about 90°, about 100°, about 110°, about 120°, about 130°, about 135°, about 140°, about 150°, or about 160°, etc.

In some cases, there may be a change in the dimensions of the main channel as side channels 45 intersect. In this figure, upon intersection of the side channels, the main channel increases in cross-sectional area. The change in area may be effected by a change in any dimension, e.g., width, length, or both, depending on the embodiment. In other cases, however, the main channel may not necessarily change in cross-sectional area.

In some embodiments, gases entering from a side channel may be dried and/or heated, which may facilitate drying of liquids or other fluids within the main channel. For example, the gases may be introduced to the liquids or other fluids at a temperature of at least about 40° C., at least about 50° C., at least about 60° C., at least about 70° C., at least about 80° C., at least about 90° C., etc. The gases may be introduced from one or more suitable sources. One or more than one gas may be used, e.g., introduced through one or more channels. In addition, the same or different gases may be introduced through the various side channels. In some embodiments, the entering gases may be relatively unsaturated with an evaporating liquid, thereby allowing the liquid within the channel to continue dry without saturation of the gas within the channel with evaporated liquid. In some cases, air or other gases that are at least partially saturated with solvent or other fluid from the droplets may be quickly brought to an outlet and replaced by “dry” air or gases that are relatively unsaturated. The gas may be any suitable gas, for example, air, nitrogen, argon, carbon dioxide, helium, etc., as well as combinations of these and/or other gases. The gas may be at ambient pressure, or the gas may be pressurized in some instances. For instance, the pressure of the incoming gas may be at least about 0.01 bar, at least about 0.03 bar, at least about 0.05 bar, at least about 0.07 bar, at least about 0.1 bar, at least about 0.2 bar, at least about 0.3 bar, at least about 0.4 bar, at least about 0.5 bar, at least about 0.7 bar, at least about 1 bar, at least about 2 bar, at least about 3 bar, at least about 4 bar, at least about 5 bar, at least about 6 bar, at least about 8 bar, at least about 10 bar, at least about 12 bar, at least about 15 bar, at least about 18 bar, at least about 20 bar, etc. In some cases, the gases are inert relative to the fluids and/or species contained therein.

In addition, in one set of embodiments, liquids or other fluids within a channel may be prevented from coming into contact with a wall of the channel, or at least a portion of the channel. In some embodiments, the liquid is prevented from coming into contact with a wall of the channel substantially throughout the length of the channel. In addition, in some cases, one or more walls or regions within the channel may be chemically treated, e.g., as discussed herein. By preventing the droplets from contacting the walls of the channel, reactions or interactions between a fluid and the walls of the channel may be reduced or eliminated. For instance, the fluid may contain a species (e.g., dissolved or suspended therein) that is able to bind to (or “foul”) a wall of the channel if the species comes into contact with the wall; by preventing, reducing, or minimizing contact between the fluid and the wall, the ability of the species to bind to the wall is reduced or eliminated. Such binding may be specific or non-specific. Examples include, but are not limited to, chemical modification groups such as perfluorinated silanes, hydrocarbon-based silanes, poly(ethylene glycol)-based silanes, polyelectrolytes, polyelectrolyte multilayers, parylene, SiO₂ produced through sol-gel methods, and the like. Examples of sol-gel and other coating methods are described in more detail herein.

In some embodiments, liquids or other fluids within a channel may be prevented from coming into contact with a wall of the channel based on the channel dimensions or geometry. For example, upon intersection of one or more side channels to the main channel, the main channel may exhibit an increase or a decrease in cross-sectional area. For instance, the main channel may exhibit a change in any dimension, e.g., width, length, or both.

Another aspect of the present invention is generally directed to systems and methods for accelerating a fluid within a channel, such as a microfluidic channel. This may occur in a spray dryer, or in other systems or devices (e.g., any suitable microfluidic device) in some cases, not necessarily only in spray dryers. For instance, a fluid within a channel (e.g., present as droplets, a jet, a film, etc.) may be accelerated by the entering gases, which may cause the fluid to flow faster within the channel in some embodiments, and optionally such that the fluid becomes disrupted or dispersed to form smaller droplets. Other methods of accelerating a fluid within a channel are also possible, for example, electrical or magnetic techniques.

The average velocity of the fluid within the channel may be increased by at least about 5%, at least about 10%, at least about 20%, at least about 30%, at least about 50%, at least about 75%, at least about 100%, etc., using techniques such as those described herein. In addition, even higher increases in velocity may be achieved in certain embodiments, for example, the fluid velocity may be accelerated by a factor of at least about 2 times, at least about 3 times, at least about 5 times, at least about 7 times, at least about 10 times, at least about 20 times, at least about 30 times, at least about 50 times, at least about times, at least about 70 times, at least about 100 times, at least about 200 times, at least about 300 times, at least about 500 times, at least about 700 times, at least about 1000 times, at least about 2000 times, at least about 3000 times, etc. In some cases, the average velocity may be increased to at least about 1 m/s, at least about 2 m/s, at least about 3 m/s, at least about 5 m/s, at least about 7 m/s, at least about 10 m/s, at least about 20 m/s, at least about 30 m/s, at least about 40 m/s, at least about 50 m/s, at least about 60 m/s, at least about 70 m/s, etc.

This increase in average velocity of the fluid can be determined relative to the average velocity of the fluid before the gas is introduced into the microfluidic channel. In some embodiments, the channel may be formed from materials that are relatively inelastic and unable to expand (although in some cases, the channel may be formed from materials that allow some expansion to occur, e.g., homogenously). Accordingly, under such conditions, the flow of the fluid within the channel may increase as gases enter the channel, e.g., at one or more locations within the channel, thereby causing the fluid to flow or move faster within the channel.

In addition, under some conditions, the increased velocity may create shear forces on the fluid, and may in some cases cause the fluid to become disrupted, thereby forming smaller droplets within the channel. For example, the forces applied to the droplets may be such that the inertial forces overcome the surface tension forces within the droplets. Smaller droplets may also facilitate drying of the fluidic droplet or evaporation of liquid, prior to being sprayed into the collection region. Thus, as a non-limiting example, a fluid droplet or film may be disrupted or dispersed to form smaller droplets by accelerating the fluid within the channel. For example, smaller droplet sizes would result in greater surface area and a smaller volume-to-area area ratio for the smaller droplets, thereby promoting additional drying.

In some embodiments of the invention, other materials instead of and/or in addition to gases may be introduced through one or more of the side channels. Examples of other materials that may be introduced include, for example, particles (e.g., to disrupt fluids within the channel), additional fluids, other reactants (e.g., able to react with a fluid and/or species contained within a fluid), other liquids or materials for introduction into or association with the final dried solid material, or the like. For example, in one set of embodiments, excipients or other materials, such as salts, carriers, buffering agents, emulsifiers, diluents, chelating agents, fillers, drying agents, antioxidants, antimicrobials, preservatives, binding agents, bulking agents, silicas, solubilizers, or stabilizers, may be introduced.

In certain embodiments, liquid droplets within a channel (e.g., prior to being expelled) may be dried to the point where the liquid becomes saturated or supersaturated with a species contained therein. In certain cases, supersaturated droplets may be expelled at a surface, e.g., of a collection chamber, and one or more particles may form upon impacting the surface. In other embodiments, however, the supersaturated droplets may solidify prior to being expelled into a drying or collection region, e.g., to form one or more particles.

Additionally, in accordance with some aspects, there may be one or more openings on nozzles in one or more of the channels that are used to expel droplets and/or particles into a collection region, or into more than one collection region in some cases. The openings can be, for instance, a simple opening or a hole in the side of a channel, an open end of a channel, or there may be an additional structure associated with the opening that the droplets and/or particles pass through before being expelled into a drying region, for example, a pipe or a tube having varying cross sectional area that can be used to direct or modify the flow of the fluid. The opening can act as a nozzle through which a droplets and/or particles can be expelled from the channel into the drying region. The opening or nozzle may have a cross-sectional aspect ratio that is the same or different from the channel. In some cases, the cross-sectional aspect ratio of the opening or nozzle may be about 1:1, at least about 1:1, at least about 2:1, at least about 3:1, at least about 4:1, at least about 5:1, at least about 6:1, at least about 7:1, at least about 8:1, at least about 10:1, at least about 12:1, at least about 15:1, or at least about 20:1. In some cases, the opening may be constructed and arranged to cause a fluid to form a spray or a mist of droplets. In other embodiments, the droplets can be expelled as a regular or steady stream of droplets and/or particles, e.g., a single file stream of droplets.

In some cases, one or more gases may be delivered to cause a fluid to break up into discrete droplets upon expulsion of the fluid into the collection region, and in some cases, such that a spray or a mist of droplets is formed. Without wishing to be bound by any theory, it is believed that fluid break-up can occur if the droplets experience forces such that the inertial forces exceed the surface tension forces, i.e., the external forces felt by the fluidic droplet exceed the inherent ability of the fluid to keep itself together as a droplet under surface tension. In addition, in some embodiments, the droplets can form through Rayleigh-Plateau instabilities or absolute instabilities. In many cases, the higher the acceleration felt by the droplet, the smaller the droplets that are subsequently formed after break-up. This may also accelerate solvent evaporation, since solvent evaporation is typically proportional to the exposed surface area. For example, the gas may be any of the gases described herein, and at any of the pressures described herein. The gas may be the same or different than other gases within the channel (e.g., used to cause acceleration and/or drying within the channel).

Thus, the droplets and/or particles formed from solidifying droplets (completely or partially solidified) may then be sprayed (or spray-dried), or otherwise expelled, into a suitable collection region. The collection region may be open, e.g., open to the atmosphere, or closed, for example, partially or completely surrounded by a chamber into which the droplets and/or particles are expelled. For example, a collection chamber can be formed of glass, plastic, or any other suitable material which can be used to at least partially contain or enclose a suitable drying gas for drying fluids expelled into the collection region. The collection region may have any suitable volume. The drying gas may be air, nitrogen, carbon dioxide, argon, oxygen, or other suitable gases. In some embodiments, the gas is chosen so as to be relatively inert or unreactive to the expelled fluids or other materials; however, in other embodiments, the gas may react with one or more of the expelled fluids or other materials. The drying gas can also be dehumidified using various techniques, for example, refrigeration or condensing cycles, electronic methods (e.g., Peltier heat pumps), desiccants (e.g., phosphorus pentoxide), or hygroscopic materials. In some embodiments, the relative humidity within the collection region is no more than about 50%, no more than about 40%, no more than about 35%, no more than about 30%, no more than about 25%, no more than about 20%, no more than about 15%, no more than about 10%, or no more than about 5%. Other techniques for controlling the relative humidity of a region will be known to those of ordinary skill in the art.

In some cases, the collection region is heated, e.g., using one or more heaters. The temperature of the collection region may be chosen, for example, to allow partial or complete drying of the expelled fluids or other materials to occur (depending on the application), in some cases without causing adverse degradation or reaction with the expelled fluids or other materials. For example, the heater may be used to heat the collection region to a temperature of at least about 30° C., at least about 40° C., at least about 60° C., at least about 80° C., at least about 100° C., at least about 125° C., at least about 150° C., at least about 200° C., at least about 300° C., at least about 400° C., at least about 500° C., etc. Any suitable method may be used to heat the collection region. For example, the collection region may be heated using induction heating, burning of a fuel, exposure to radiation (e.g., infrared radiation), chemical reaction, or the like.

In some cases, a population of droplets is formed upon expulsion of fluids from the channel into the collection region. The average diameter of this population may or may not necessarily be the same as the average droplets within the channel, prior to being expelled into the collection region. Those of ordinary skill in the art will be able to determine the average diameter of a population of droplets, for example, using laser light scattering or other known techniques. The droplets so formed can be spherical, or non-spherical in certain cases. The diameter of a droplet, in a non-spherical droplet, may be taken as the diameter of a perfect mathematical sphere having the same volume as the non-spherical droplet. The droplets may be formed steadily, for example, forming a steady or linear stream of droplets, or in other embodiments, larger numbers of droplets may be formed, for example, creating a mist or a spray of individual droplets, e.g., within the collection region.

In some cases, as previously discussed, liquid may evaporate from the droplets, which may cause the average diameter of the droplets to decrease in some embodiments. In certain embodiments, as non-limiting examples, the average diameter of the droplets can be less than about 1 mm, less than about 500 micrometers, less than about 300 micrometers, less than about 200 micrometers, less than about 100 micrometers, less than about 75 micrometers, less than about 50 micrometers, less than about 30 micrometers, less than about 25 micrometers, less than about 20 micrometers, less than about 15 micrometers, less than about 10 micrometers, less than about 5 micrometers, less than about 3 micrometers, less than about 2 micrometers, less than about 1 micrometer, less than about 500 nm, less than about 300 nm, less than about 100 nm, or less than about 50 nm. The average diameter of the droplets may also be at least about 30 nm, at least about 50 nm, at least about 100 nm, at least about 300 nm, at least about 500 nm, at least about 1 micrometer, at least about 2 micrometers, at least about 3 micrometers, at least about 5 micrometers, at least about 10 micrometers, at least about 15 micrometers, or at least about 20 micrometers in certain cases.

In certain embodiments, the fluidic droplets within the collection region, e.g., after being expelled from a channel, may be substantially monodisperse. For example, the fluidic droplets may have a distribution in diameters such that no more than about 5%, no more than about 2%, or no more than about 1% of the droplets have a diameter less than about 90% (or less than about 95%, or less than about 99%) and/or greater than about 110% (or greater than about 105%, or greater than about 101%) of the overall average diameter of the plurality of droplets. However, in other embodiments, the fluidic droplets within the collection region are polydisperse.

Other aspects of the present invention include the following. Certain embodiments of the present invention present a versatile tool, e.g., for the development of new formulations. For example, small quantities of a drug, pharmaceutical agent, or other species (e.g., as discussed herein) can be tested in some cases. In certain embodiments, for instance, a drug, pharmaceutical agent, or other species may be tested for its spray drying characteristics relatively rapidly, and/or without requiring a large initial amount of sample for testing purposes. Conditions for spray drying may be changed relatively rapidly, e.g., before and/or during spray drying experiments, in order to experiment or optimize various formulations, and in some cases without requiring a relatively large amount of drug, pharmaceutical agent, or other species. For instance, no more than about 100 g, no more than about 50 g, no more than about 30 g, no more than about 10 g, no more than about 5 g, no more than about 3 g, no more than about 1 g, no more than about 500 mg, no more than about 300 mg, or no more than about 100 mg of drug, pharmaceutical agent, or other species may be used in the spray dryer in certain embodiments, e.g., to produce particles. In some cases, relatively small numbers or masses of particles may be produced in a given spray drying experiment, e.g., allowing conditions to be rapidly changed, for example, as discussed above. For instance, no more than about 100 g, no more than about 50 g, no more than about 30 g, no more than about 10 g, no more than about 5 g, no more than about 3 g, no more than about 1 g, no more than about 500 mg, no more than about 300 mg, or no more than about 100 mg of particles or solids may be formed using the spray dryer. In some cases, the composition of the particles may be easily controlled, e.g., by controlling fluid flow into the spray dryer, and/or by joining two or more different fluid streams containing different dissolved substances into one, e.g., just before droplet formation.

In addition, in some embodiments, a spray dryer as discussed herein may have a relatively low dead volume, which may thus reduce waste of sample and/or facilitate experiments that use minimal amounts of drugs, pharmaceutical agents, or other species. The dead volume of the spray dryer includes volumes within the spray dryer which contain volumes of fluid that are not able to be expelled by the spray dryer into the drying region during normal operation of the spray dryer.

In some cases, a suspension may be produced using spray dryers such as those discussed herein. Such suspensions may be used, for example, to enhance the dissolution rate and bioavailability of hydrophobic drugs. For instance, a suspension can be prepared by spraying a fluid into a carrier liquid. In some embodiments, the carrier liquid may contain a stabilizer or a surfactant, e.g., as in a solution. In other embodiments, however, no stabilizer or surfactant may be present in the carrier liquid. In some cases, the fluid being expelled may be dried sufficiently to produce particles prior to contacting the carrier liquid; in other cases, however, the fluids may enter the solution not fully dried, for example, to form a liquid suspension in the carrier liquid.

In addition, in some embodiments, a spray dryer may be directly connected to a vial, a sample holder, an ampoule, etc., without necessarily requiring intermediate processing and/or storage, for example, fluid transport or filling from a collection chamber to a vial, which can cause waste, alteration of physical or chemical properties, etc. For example, one or more relatively small vials (or other collection chambers) may be used to directly collect material produced by the spray dryer. The vial or other collection chamber may have a relatively small volume, e.g., less than about 100 ml, less than about 50 ml, less than about 30 ml, less than about 20 ml, less than about 15 ml, less than about 10 ml, less than about 5 ml, etc. In some cases, one collection chamber is used, although in other cases, more than one may be used, e.g., such that one is replaced by the next (manually or automatically) after a certain time and/or after a certain amount has been collected therein.

As mentioned, in various aspects of the invention, liquid droplets may pass through channels, and gases may also be introduced into the channel through side channels. The main channel and the side channels may be the same size or different, and one or both may be microfluidic channels. These channels may be relatively straight, e.g., as is depicted in FIG. 1, or one or more of the channels may be bent, curved, wiggly, etc., depending on the application. In various embodiments, the channels may exhibit a constant cross-sectional shape or area, or one that varies, e.g., one that increases or decreases in area downstream. In addition, there can be any number of channels present within an article, and the channels may be arranged in any suitable configuration. The channels may be all interconnected, or there can be more than one network of channels present.

Thus, as a non-limiting example, FIG. 1 illustrates a first (main) channel, and second, third, fourth, fifth, sixth, seventh, eighth, ninth, tenth, and eleventh side channels intersecting the first channel at various intersections, i.e., second and third channels at a first intersection, fourth and fifth channels at a second intersection, sixth and seventh channels at a third intersection, eighth and ninth channels at a fourth intersection, and tenth and eleventh channels at a fifth intersection. As previously mentioned, this is by way of illustration only, and in other embodiments of there may be more or few side channels present, and their configuration (e.g., angle of intersection, orientation, numbers present at an intersection, etc.) may vary.

Fluids (e.g., liquids, gases, etc., such as those described herein) may be delivered into channels such as those described above from one or more fluid sources. Any suitable source of fluid can be used, and in some cases, more than one source of fluid is used. For example, a pump, gravity, capillary action, surface tension, electroosmosis, centrifugal forces, etc. may be used to deliver a fluid from a fluid source into one or more channels in the article. Non-limiting examples of pumps include syringe pumps, peristaltic pumps, pressurized fluid sources, or the like. The article can have any number of fluid sources associated with it, for example, 1, 2, 3, 4, 5, 6, 7, 8, 9, 10, etc., or more fluid sources. The fluid sources need not be used to deliver fluid into the same channel, e.g., a first fluid source can deliver a first fluid to a first channel while a second fluid source can deliver a second fluid to a second channel, etc.

In some embodiments, the fluids flow through the channel at relatively high flow rates or speeds, for example. The flow within the channels can be laminar or turbulent. In some cases, flow through the channel occurs such that the Reynolds number of the flow is at least about 0.001, at least about 0.003, at least about 0.005, at least about 0.01, at least about 0.03, at least about 0.05, at least about 0.1, at least about 0.3, or at least about 0.5. Higher Reynolds numbers may be used in other embodiments (e.g., corresponding to turbulent flow), for instance, Reynolds numbers of at least about 1, at least about 3, at least about 5, at least about 10, at least about 30, at least about 50, at least about 100, at least about 300, at least about 500, at least about 1000, at least about 3000, at least about 5000, at least about 10,000, at least about 20,000, at least about 30,000, at least about 40,000, at least about 50,000, etc. In still other embodiments, however, flow through the channel may occur such that the Reynolds number of the flow is less than about 50,000, less than about 40,000, less than about 30,000, less than about 20,000, less than about 10,000, less than about 5000, less than about 3000, less than about 2000, less than about 1000, less than about 300, less than about 100, less than about 30, less than about 10, less than about 3, or less than about 1. In yet other embodiments of the invention, the volumetric flow rate of fluid through the channel may be at least about 0.01 ml/h at least about 0.03 ml/h, at least about 0.05 ml/h, at least about 0.1 ml/h, at least about 0.3 ml/h, at least about 0.5 ml/h, at least about 1 ml/h, at least about 3 ml/h, at least about 5 ml/h, at least about 10 m/l, at least about 30 ml/h, at least about 50 ml/h, or at least about 100 ml/h.

Relatively high flow rates may be achieved, for example, by increasing or controlling the difference in pressure between one or more of the fluid sources within the article containing channels, and the pressure within the drying region of the spray dryer, and/or through parallelization. For example, the pressure within the drying region may be at ambient pressure (approximately 1 atm), and/or the pressure may be higher or lower. As specific non-limiting examples, the pressure within the drying region may be less than about 50 mmHg, less than about 100 mmHg, less than about 150 mmHg, less than about 200 mmHg, less than about 250 mmHg, less than about 300 mmHg, less than about 350 mmHg, less than about 400 mmHg, less than about 450 mmHg, less than about 500 mmHg, at least 550 mmHg, at least 600 mmHg, at least 650 mmHg, less than about 700 mmHg, or less than about 750 mmHg below atmospheric pressure. As another example, the pressure of one or more of the fluid sources within the article may be at least about 1 bar, at least about 1.1 bars, at least about 1.2 bars, at least about 1.3 bars, at least about 1.4 bars, at least about 1.5 bars, at least about 1.7 bars, at least about 2 bars, at least about 2.5 bars, at least about 3 bars, at least about 4 bars, at least about 5 bars, etc.

In some embodiments, at least some of the channels within the article are microfluidic channels. “Microfluidic,” as used herein, refers to a device, article, or system including at least one fluid channel having a cross-sectional dimension of less than about 1 mm. The “cross-sectional dimension” of the channel is measured perpendicular to the direction of net fluid flow within the channel. Thus, for example, some or all of the fluid channels in an article can have a maximum cross-sectional dimension less than about 2 mm, and in certain cases, less than about 1 mm. In one set of embodiments, all fluid channels in an article are microfluidic and/or have a largest cross sectional dimension of no more than about 2 mm or about 1 mm. In certain embodiments, the fluid channels may be formed in part by a single component (e.g. an etched substrate or molded unit). Of course, larger channels, tubes, chambers, reservoirs, etc. can be used to store fluids and/or deliver fluids to various elements or systems in other embodiments of the invention. In one set of embodiments, the maximum cross-sectional dimension of the channels in an article is less than about 1 mm, less than about 500 micrometers, less than about 300 micrometers, less than about 200 micrometers, less than about 100 micrometers, less than about 75 micrometers, less than about 50 micrometers, less than about 30 micrometers, less than about 25 micrometers, less than about 20 micrometers, less than about 15 micrometers, less than about 10 micrometers, less than about 5 micrometers, less than about 3 micrometers, less than about 2 micrometers, less than about 1 micrometer, less than about 500 nm, less than about 300 nm, less than about 100 nm, or less than about 50 nm.

A channel can have any cross-sectional shape (circular, oval, triangular, irregular, square or rectangular, or the like) and can be covered or uncovered. In embodiments where it is completely covered, at least one portion of the channel can have a crosssection that is completely enclosed, or the entire channel may be completely enclosed along its entire length with the exception of its inlets and/or outlets or openings. An open channel generally will include characteristics that facilitate control over fluid transport, e.g., structural characteristics (an elongated indentation) and/or physical or chemical characteristics (hydrophobicity vs. hydrophilicity) or other characteristics that can exert a force (e.g., a containing force) on a fluid. The fluid within the channel may partially or completely fill the channel. In some cases where an open channel is used, the fluid may be held within the channel, for example, using surface tension (i.e., a concave or convex meniscus).

The channel may be of any size, for example, having a largest dimension perpendicular to net fluid flow of less than about 5 mm or 2 mm, or less than about 1 mm, less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm. In some cases, the dimensions of the channel are chosen such that fluid is able to freely flow through the article or substrate. The dimensions of the channel may also be chosen, for example, to allow a certain volumetric or linear flow rate of fluid in the channel. Of course, the number of channels and the shape of the channels can be varied by any method known to those of ordinary skill in the art. In some cases, more than one channel may be used. For example, two or more channels may be used, where they are positioned adjacent or proximate to each other, positioned to intersect with each other, etc.

In one set of embodiments, the channels within the article are arranged in a quasi-2-dimensional pattern. In a “quasi-2-dimensional pattern,” the channels within the article are constructed and arranged such that at least one plane can be defined relative to the article such that, when all of the channels within the article are “shadowed” or perpendicularly projected onto the plane, any two channels that appear to be fluidically connected are, in fact, fluidically connected (i.e., there are no “bridges” within the article separating those fluids in separate channels). Such articles are useful in certain cases, for example, due to their ease of manufacturing, creation, or preparation.

In certain embodiments, one or more of the channels within the article may have an average cross-sectional dimension of less than about 10 cm. In certain instances, the average cross-sectional dimension of the channel is less than about 5 cm, less than about 3 cm, less than about 1 cm, less than about 5 mm, less than about 3 mm, less than about 1 mm, less than 500 micrometers, less than 200 micrometers, less than 100 micrometers, less than 50 micrometers, or less than 25 micrometers. The “average cross-sectional dimension” is measured in a plane perpendicular to net fluid flow within the channel. If the channel is non-circular, the average cross-sectional dimension may be taken as the diameter of a circle having the same area as the cross-sectional area of the channel. Thus, the channel may have any suitable cross-sectional shape, for example, circular, oval, triangular, irregular, square, rectangular, quadrilateral, or the like. In some embodiments, the channels are sized so as to allow laminar flow of one or more fluids contained within the channel to occur.

The channel may also have any suitable cross-sectional aspect ratio. The “cross-sectional aspect ratio” is, for the cross-sectional shape of a channel, the largest possible ratio (large to small) of two measurements made orthogonal to each other on the cross-sectional shape. For example, the channel may have a cross-sectional aspect ratio of less than about 2:1, less than about 1.5:1, or in some cases about 1:1 (e.g., for a circular or a square cross-sectional shape). In other embodiments, the cross-sectional aspect ratio may be relatively large. For example, the cross-sectional aspect ratio may be at least about 2:1, at least about 3:1, at least about 4:1, at least about 5:1, at least about 6:1, at least about 7:1, at least about 8:1, at least about 10:1, at least about 12:1, at least about 15:1, or at least about 20:1. Relatively large cross-sectional aspect ratios are useful in accordance with some embodiments, as is discussed herein, for preventing or minimizing contact between a fluid within a channel and one or more walls within the channel.

As mentioned, the channels can be arranged in any suitable configuration within the article. Different channel arrangements may be used, for example, to manipulate fluids, droplets, and/or other species within the channels. For example, channels within the article can be arranged to create droplets (e.g., discrete droplets, single emulsions, double emulsions or other multiple emulsions, etc.), to mix fluids and/or droplets or other species contained therein, to screen or sort fluids and/or droplets or other species contained therein, to split or divide fluids and/or droplets, to cause a reaction to occur (e.g., between two fluids, between a species carried by a first fluid and a second fluid, or between two species carried by two fluids to occur), or the like. As a specific non-limiting example, two or more channels can be arranged to cause “flow-focusing” of different fluids within the channels to form droplets.

In some cases, there are a relatively large number and/or a relatively large length of channels present in the article. For example, in some embodiments, the channels within an article, when added together, can have a total length of at least about 100 micrometers, at least about 300 micrometers, at least about 500 micrometers, at least about 1 mm, at least about 3 mm, at least about 5 mm, at least about 10 mm, at least about 30 mm, at least 50 mm, at least about 100 mm, at least about 300 mm, at least about 500 mm, at least about 1 m, at least about 2 m, or at least about 3 m in some cases. As another example, an article can have at least 1 channel, at least 3 channels, at least 5 channels, at least 10 channels, at least 20 channels, at least 30 channels, at least 40 channels, at least 50 channels, at least 70 channels, at least 100 channels, etc.

The channel may also be coated in some embodiments. For example, the coating may render the walls (or a portion thereof) of the channel more hydrophobic or more hydrophilic, depending on the application. As a specific non-limiting example, a fluid may be relatively hydrophilic and the channel walls may be relatively hydrophobic, and/or coated to render the walls more hydrophobic, such that the fluid is generally repelled (does not wet) the walls of the channel, thereby assisting in preventing the fluid from contacting the hydrophobic walls defining the fluidic channel. Such a configuration may be useful, for instance, for droplet formation. In some embodiments, for example, for film formation, the channel walls may be chosen to be relatively hydrophilic (e.g., for a relatively hydrophilic fluid) or relatively hydrophobic (e.g., for a relatively hydrophobic fluid).

As yet another example, the fluid may be relatively hydrophobic and the channel walls may be relatively hydrophilic. Typically, a “hydrophilic” material or surface is one that wets water, e.g., water on such a surface has a contact angle of less than 90°, while a “hydrophobic” material or surface has a contact angle of greater than 90°. However, hydrophobicity may also be determined in other embodiments in a relative sense, i.e., a first material may be more hydrophilic than a second material (e.g., have a smaller contact angle), although the materials may both be hydrophilic or both be hydrophobic.

Any suitable method may be used to coat or treat the walls (or a portion thereof) of a channel. For instance, a wall can be treated with oxygen plasma treatment, or coated with a sol-gel material, a silane, a polyelectrolyte, parylene, etc. that can be used to alter the hydrophobicity of the wall and/or to render the walls chemically more inert, etc. A portion of the sol-gel may be exposed to light, such as ultraviolet light, which can be used to induce a chemical reaction in the sol-gel that alters its hydrophobicity. The sol-gel can include a photoinitiator which, upon exposure to light, produces radicals. Optionally, the photoinitiator is conjugated to a silane or other material within the sol-gel. The radicals so produced may be used to cause a condensation or polymerization reaction to occur on the surface of the sol-gel, thus altering the hydrophobicity of the surface. As another non-limiting example, a metal oxide may be coated onto a wall to alter its hydrophobicity. Still other examples are disclosed below, and in International Patent Application No. PCT/US2009/000850, filed Feb. 11, 2009, entitled “Surfaces, Including Microfluidic Channels, With Controlled Wetting Properties,” by Abate, et al., published as WO 2009/120254 on Oct. 1, 2009, and U.S. patent application Ser. No. 12/733,086, filed Feb. 5, 2010, entitled “Metal Oxide Coating on Surfaces,” by Weitz, et al., published as U.S. Patent Application Publication No. 2010/0239824 on Sep. 23, 2010, each of which is incorporated herein by reference in its entirety.

Non-limiting examples of systems for manipulating fluids, droplets, and/or other species are discussed below. Additional examples of suitable manipulation systems can also be seen in U.S. patent application Ser. No. 11/246,911, filed Oct. 7, 2005, entitled “Formation and Control of Fluidic Species,” by Link, et al., published as U.S. Patent Application Publication No. 2006/0163385 on Jul. 27, 2006; U.S. patent application Ser. No. 11/024,228, filed Dec. 28, 2004, entitled “Method and Apparatus for Fluid Dispersion,” by Stone, et al., now U.S. Pat. No. 7,708,949, issued May 4, 2010; U.S. patent application Ser. No. 11/885,306, filed Aug. 29, 2007, entitled “Method and Apparatus for Forming Multiple Emulsions,” by Weitz, et al., published as U.S. Patent Application Publication No. 2009/0131543 on May 21, 2009; and U.S. patent application Ser. No. 11/360,845, filed Feb. 23, 2006, entitled “Electronic Control of Fluidic Species,” by Link, et al., published as U.S. Patent Application Publication No. 2007/0003442 on Jan. 4, 2007; each of which is incorporated herein by reference in its entirety.

A variety of materials and methods, according to certain aspects of the invention, can be used to form articles or components such as those described herein, e.g., channels such as microfluidic channels, chambers, etc. For example, various articles or components can be formed from solid materials, in which the channels can be formed via micromachining, film deposition processes such as spin coating and chemical vapor deposition, laser fabrication, photolithographic techniques, etching methods including wet chemical or plasma processes, 3D printing, and the like. See, for example, Scientific American, 248:44-55, 1983 (Angell, et al).

In one set of embodiments, various structures or components of the articles described herein can be formed of a polymer, for example, an elastomeric polymer such as polydimethylsiloxane (“PDMS”), polytetrafluoroethylene (“PTFE” or Teflon®), epoxy, norland optical adhesive, or the like. For instance, according to one embodiment, a microfluidic channel may be implemented by fabricating the fluidic system separately using PDMS or other soft lithography techniques (details of soft lithography techniques suitable for this embodiment are discussed in the references entitled “Soft Lithography,” by Younan Xia and George M. Whitesides, published in the Annual Review of Material Science, 1998, Vol. 28, pages 153-184, and “Soft Lithography in Biology and Biochemistry,” by George M. Whitesides, Emanuele Ostuni, Shuichi Takayama, Xingyu Jiang and Donald E. Ingber, published in the Annual Review of Biomedical Engineering, 2001, Vol. 3, pages 335-373; each of these references is incorporated herein by reference).

Other examples of potentially suitable polymers include, but are not limited to, polyethylene terephthalate (PET), polyacrylate, polymethacrylate, polycarbonate, polystyrene, polyethylene, polypropylene, polyvinylchloride, cyclic olefin copolymer (COC), polytetrafluoroethylene, a fluorinated polymer, a silicone such as polydimethylsiloxane, polyvinylidene chloride, bis-benzocyclobutene (“BCB”), a polyimide, a fluorinated derivative of a polyimide, or the like. Combinations, copolymers, or blends involving polymers including those described above are also envisioned. The device may also be formed from composite materials, for example, a composite of a polymer and a semiconductor material.

In some embodiments, various structures or components of the article are fabricated from polymeric and/or flexible and/or elastomeric materials, and can be conveniently formed of a hardenable fluid, facilitating fabrication via molding (e.g. replica molding, injection molding, cast molding, etc.). The hardenable fluid can be essentially any fluid that can be induced to solidify, or that spontaneously solidifies, into a solid capable of containing and/or transporting fluids contemplated for use in and with the fluidic network. In one embodiment, the hardenable fluid comprises a polymeric liquid or a liquid polymeric precursor (i.e. a “prepolymer”). Suitable polymeric liquids can include, for example, thermoplastic polymers, thermoset polymers, waxes, metals, or mixtures or composites thereof heated above their melting point. As another example, a suitable polymeric liquid may include a solution of one or more polymers in a suitable solvent, which solution forms a solid polymeric material upon removal of the solvent, for example, by evaporation. Such polymeric materials, which can be solidified from, for example, a melt state or by solvent evaporation, are well known to those of ordinary skill in the art. A variety of polymeric materials, many of which are elastomeric, are suitable, and are also suitable for forming molds or mold masters, for embodiments where one or both of the mold masters is composed of an elastomeric material. A non-limiting list of examples of such polymers includes polymers of the general classes of silicone polymers, epoxy polymers, and acrylate polymers. Epoxy polymers are characterized by the presence of a three-membered cyclic ether group commonly referred to as an epoxy group, 1,2-epoxide, or oxirane. For example, diglycidyl ethers of bisphenol A can be used, in addition to compounds based on aromatic amine, triazine, and cycloaliphatic backbones. Another example includes the well-known Novolac polymers. Non-limiting examples of silicone elastomers suitable for use according to the invention include those formed from precursors including the chlorosilanes such as methylchlorosilanes, ethylchlorosilanes, phenylchlorosilanes, dodecyltrichlorosilanes, etc.

Silicone polymers are used in certain embodiments, for example, the silicone elastomer polydimethylsiloxane. Non-limiting examples of PDMS polymers include those sold under the trademark Sylgard by Dow Chemical Co., Midland, Mich., and particularly Sylgard 182, Sylgard 184, and Sylgard 186. Silicone polymers including PDMS have several beneficial properties simplifying fabrication of various structures of the invention. For instance, such materials are inexpensive, readily available, and can be solidified from a prepolymeric liquid via curing with heat. For example, PDMSs are typically curable by exposure of the prepolymeric liquid to temperatures of about, for example, about 65° C. to about 75° C. for exposure times of, for example, about an hour, about 3 hours, about 12 hours, etc. Also, silicone polymers, such as PDMS, can be elastomeric and thus may be useful for forming very small features with relatively high aspect ratios, necessary in certain embodiments of the invention. Flexible (e.g., elastomeric) molds or masters can be advantageous in this regard.

One advantage of forming structures such as microfluidic structures or channels from silicone polymers, such as PDMS, is the ability of such polymers to be oxidized, for example by exposure to an oxygen-containing plasma such as an air plasma, so that the oxidized structures contain, at their surface, chemical groups capable of cross-linking to other oxidized silicone polymer surfaces or to the oxidized surfaces of a variety of other polymeric and non-polymeric materials. Thus, structures can be fabricated and then oxidized and essentially irreversibly sealed to other silicone polymer surfaces, or to the surfaces of other substrates reactive with the oxidized silicone polymer surfaces, without the need for separate adhesives or other sealing means. In most cases, sealing can be completed simply by contacting an oxidized silicone surface to another surface without the need to apply auxiliary pressure to form the seal. That is, the pre-oxidized silicone surface acts as a contact adhesive against suitable mating surfaces. Specifically, in addition to being irreversibly sealable or bonded to itself, oxidized silicone such as oxidized PDMS can also be sealed irreversibly to a range of oxidized materials other than itself including, for example, glass, silicon, silicon oxide, quartz, silicon nitride, polyethylene, polystyrene, glassy carbon, and epoxy polymers, which have been oxidized in a similar fashion to the PDMS surface (for example, via exposure to an oxygen-containing plasma). Oxidation and sealing methods useful in the context of the present invention, as well as overall molding techniques, are described in the art, for example, in an article entitled “Rapid Prototyping of Microfluidic Systems and Polydimethylsiloxane,” Anal. Chem., 70:474-480, 1998 (Duffy et al.), incorporated herein by reference.

Thus, in certain embodiments, the design and/or fabrication of the article may be relatively simple, e.g., by using relatively well-known soft lithography and other techniques such as those described herein. In addition, in some embodiments, rapid and/or customized design of the article is possible, for example, in terms of geometry. In one set of embodiments, the article may be produced to be disposable, for example, in embodiments where the article is used with substances that are radioactive, toxic, poisonous, reactive, biohazardous, etc., and/or where the profile of the substance (e.g., the toxicology profile, the radioactivity profile, etc.) is unknown. Another advantage to forming channels or other structures (or interior, fluid-contacting surfaces) from oxidized silicone polymers is that these surfaces can be much more hydrophilic than the surfaces of typical elastomeric polymers (where a hydrophilic interior surface is desired). Such hydrophilic channel surfaces can thus be more easily filled and wetted with aqueous solutions than can structures comprised of typical, unoxidized elastomeric polymers or other hydrophobic materials.

Certain aspects of the invention are generally directed to techniques for scaling up or “numbering up” devices such as those discussed herein. For example, in one set of embodiments, a channel can have more than one opening or nozzle, which may be used to expel a plurality of droplets or particles into a collection region or into more than one collection region. As another example, an article may contain more than one channel, which may be used to expel a plurality of droplets or particles into a collection region or into more than one collection region. For instance, an article can contain at least 2 channels, at least 3 channels, at least 5 channels, at least 10 channels, at least 25 channels, at least 50 channels, at least 100 channels, some or all of which channels may have on or more openings or nozzles. As yet another example, more than one article may be present, some or all of which may have at least one opening through which droplets or particles are expelled, for instance, into a collection region or into more than one collection region. For example, multiple articles may positioned next to each other, and they may be connected via one or more distribution channels. In some cases, some or all of the articles may share one or more common sources of fluid (e.g., liquids, gases, etc.), such as those described herein. As still another example, combinations of any of these may be present.

If more than one article is present, the articles may independently be substantially the same or different. In some embodiments, for instance, greater production of droplets or particles can be achieved simply by adding additional substantially identical copies of the articles used to produce the droplets or particles. For example, a spray dryer may contain at least 2 articles, at least 3 articles, at least 5 articles, at least 10 articles, at least 25 articles, at least 50 articles, at least 100 articles, at least 250 articles, at least 500 articles, at least 1000 articles, etc., which may be used to expel a plurality of droplets or particles into a collection region or into more than one collection region. The articles can draw fluids from a common fluid source or more than one common fluid source in some embodiments. In certain embodiments, for example, each article can have its own fluid source.

Those of ordinary skill in the art will be aware of other techniques useful for scaling up or numbering up devices or articles such as those discussed herein. For example, in some embodiments, a fluid distributor can be used to distribute fluid from one or more inputs to a plurality of outputs, e.g., in one more devices. For instance, a plurality of articles may be connected in three dimensions. In some cases, channel dimensions are chosen that allow pressure variations within parallel devices to be substantially reduced. Other examples of suitable techniques include, but are not limited to, those disclosed in International Patent Application No. PCT/US2010/000753, filed Mar. 12, 2010, entitled “Scale-up of Microfluidic Devices,” by Romanowsky, et al., published as WO 2010/104597 on Nov. 16, 2010, incorporated herein by reference in its entirety.

The following documents are incorporated herein by reference in their entireties: U.S. patent application Ser. No. 11/246,911, filed Oct. 7, 2005, entitled “Formation and Control of Fluidic Species,” by Link, et al., published as U.S. Patent Application Publication No. 2006/0163385 on Jul. 27, 2006; U.S. patent application Ser. No. 11/024,228, filed Dec. 28, 2004, entitled “Method and Apparatus for Fluid Dispersion,” by Stone, et al., now U.S. Pat. No. 7,708,949, issued May 4, 2010; U.S. patent application Ser. No. 11/885,306, filed Aug. 29, 2007, entitled “Method and Apparatus for Forming Multiple Emulsions,” by Weitz, et al., published as U.S. Patent Application Publication No. 2009/0131543 on May 21, 2009; U.S. patent application Ser. No. 11/360,845, filed Feb. 23, 2006, entitled “Electronic Control of Fluidic Species,” by Link, et al., published as U.S. Patent Application Publication No. 2007/0003442 on Jan. 4, 2007; International Patent Application No. PCT/US2011/001993, filed Dec. 20, 2011, entitled “Spray Drying Techniques,” by Abate, et al.; and U.S. Provisional Patent Application Ser. No. 61/704,422, filed Sep. 21, 2012, entitled “Systems and Methods for Spray Drying in Microfluidic and Other Systems,” each of which is incorporated herein by reference in its entirety. Also incorporated herein by reference in its entirety is U.S. Provisional Patent Application Ser. No. 61/897,144, filed Oct. 29, 2013, entitled “Drying Techniques for Microfluidic and Other Systems,” by Weitz, et al.

The following examples are intended to illustrate certain embodiments of the present invention, but do not exemplify the full scope of the invention.

### Example 1

The poor water solubility of many newly developed drugs and nutrition supplements limits their bioavailability and therefore effectiveness as medication. The dissolution rate of hydrophobic moieties generally increases with decreasing particle size. It is therefore often beneficial to formulate poorly water soluble active substances as nanoparticles if they are intended for applications that require fast dissolution rates.

The size of active particles can often be tuned using various formulations. Spray drying is an often-used method to formulate drug particles for oral administration and inhalation due to its high throughput and cost effectiveness. Commercial spray driers typically include a nozzle where a solvent containing dissolved actives is atomized, a drying chamber where the solvent is evaporated under a steady air flow, and a collection chamber that can optionally be electrostatically charged to increase the yield of spray dried particles.

During the spray drying process, active nanoparticles nucleate and grow inside droplets in the drying chamber. The concentration of actives inside droplets steadily increases during solvent evaporation that occurs in the drying chamber. When the active concentration reaches the saturation concentration, actives start to nucleate and grow. Particles grow until the solvent is completely evaporated. Thus, the particle size decreases with increasing solvent evaporation rates as the nanoparticle growth time is directly proportional to the solvent evaporation rate. Particles formulated using commercially available spray driers typically are 500 nm to several micrometrs in diameter.

To decrease the size of spray dried particles, solvent evaporation rates are often increased by blowing pre-heated air into the evaporation chamber. However, the use of hot air introduces the risk of thermal degradation of thermosensitive substances during the formulation process. Alternatively, the evaporation rate can be increased by decreasing the size of droplets generated at the nozzle of the spray drier; this results in a higher surface-to-volume ratio of the droplets which accelerates solvent evaporation. The drop size in conventional spray driers is determined by the nozzle design and the liquid properties; for commercial spray drier, drop sizes range from 30 micrometers to several hundred micrometers.

These examples illustrate a PDMS (polydimethylsiloxane) based microfluidic spray drier, a “nebulator,” that forms droplets within the device. Droplets are accelerated by the high velocity of the air flow that is used as a continuous phase. The high air flow rates may also lead to a further break-up of the primary droplets into smaller secondary droplets downstream the microfluidic channel. The high surface-to-volume ratio of these secondary droplets and the high convection caused by the supersonic air flow may lead to high solvent evaporation rates. The microfluidic nebulator, as shown in this example, can be used to produce non-agglomerated, amorphous hydrophobic drug and CaCO₃ nanoparticles with diameters below 30 nm.

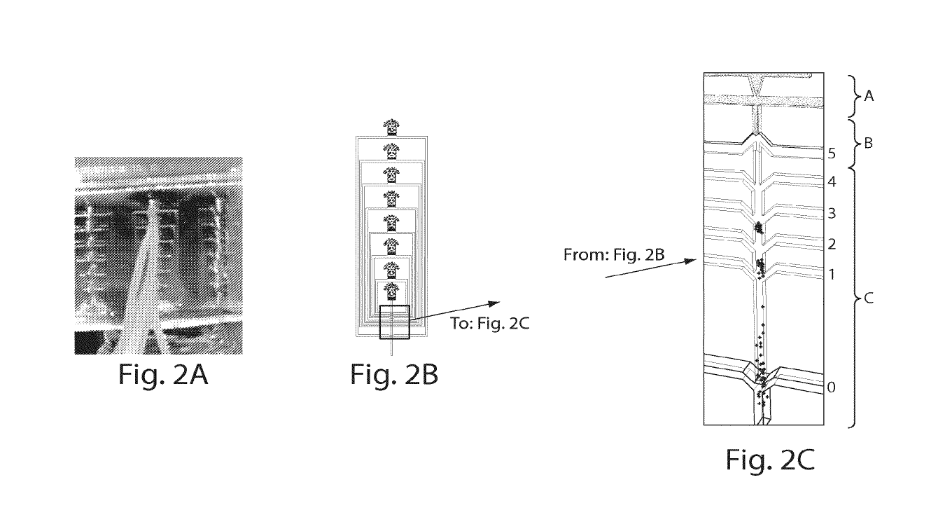

The nebulator used in this example was formed from a microfluidic PDMS device. It can be divided into three sections: (A) a liquid mixing unit where different solutions are mixed on chip, (B) followed by a nebulization unit where thin liquid films or droplets are generated, and (C) an evaporation unit where droplets are accelerated and solvents partially evaporated before they reach the device outlet (FIG. 2). The microfluidic nebulator was produced using soft lithography. To ensure a homogeneous pressure-driven expansion of all channel walls, the PDMS devices were bonded to PDMS substrates. The device nozzle was formed by slicing the device outlet with a razor blade. The PDMS channel surfaces were treated with dodecyltrichlorosilanes to render them hydrophobic. During operation, air was supplied to the nebulator through a gas regulator, and the dispersed liquid phase was fed into the microfluidic nebulator using volume controlled peristaltic pumps.

FIG. 2 shows the set-up of the microfluidic nebulator used in these examples. FIG. 2A shows the microfluidic nebulator. Air and a liquid were used as a continuous and dispersed phase, respectively. They were injected into the microfluidic device using polyethylene tubing. FIG. 2B shows an overview and FIG. 2C shows a close-up schematic of the design of the microfluidic nebulator.

### Example 2

This example describes a microfluidic spray drier, or a nebulator, that allows continuous, additive-free production of amorphous inorganic and organic nanoparticles that are below 30 nm in diameter. The nebulator allows on-chip formation of initial droplets that are broken up multiple times through the use of supersonic air flow in the final stages of the nebulator, resulting in liquid drops with sizes below 100 nm. These droplets exit the nebulator through the nozzle outlet. Fast evaporation of the liquid solvent result in rapid evaporation of the drops; this minimizes the time during which crystalline nuclei can form as droplets evaporate. In sufficiently small droplets, formation of crystalline nuclei is completely suppressed, and consequently, the resulting nanoparticles are amorphous. The nebulator therefore allows, for example, the formation of different types of additive-free inorganic and organic amorphous particles with sizes below 30 nm. In some cases, nanoparticles with a glass transition temperature T_{g} above room temperature may not crystallize if stored under ambient conditions for at least 3 weeks, making them attractive for many applications.

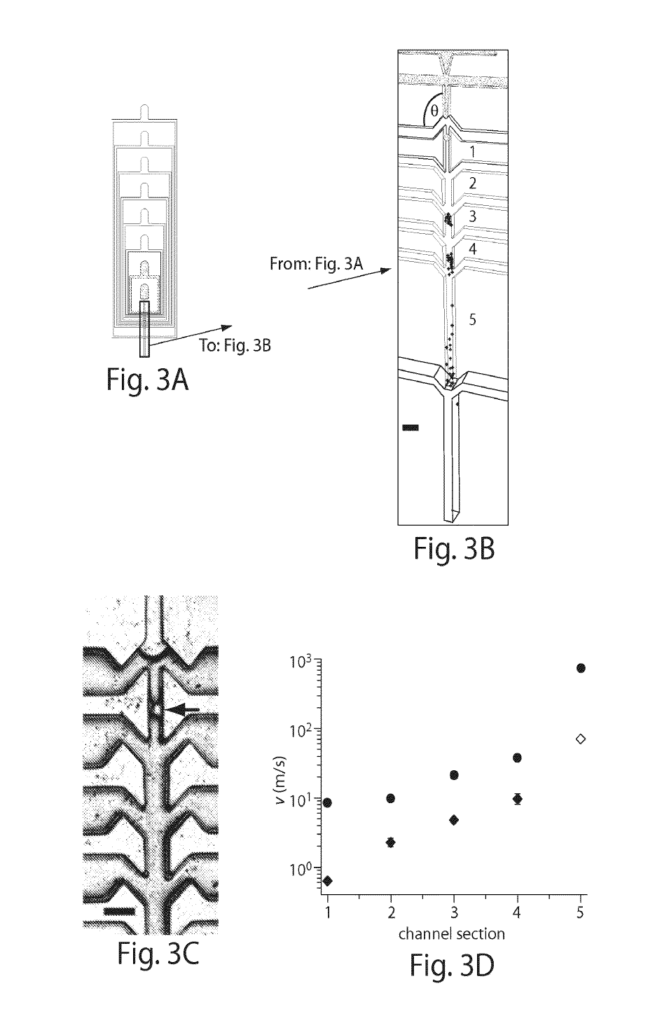

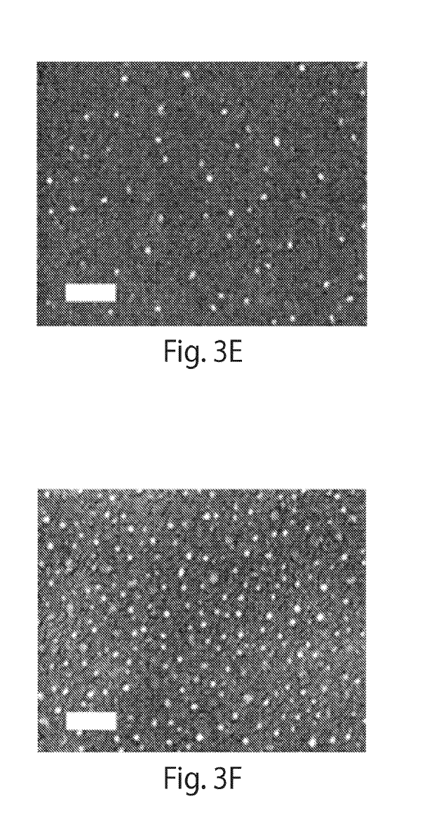

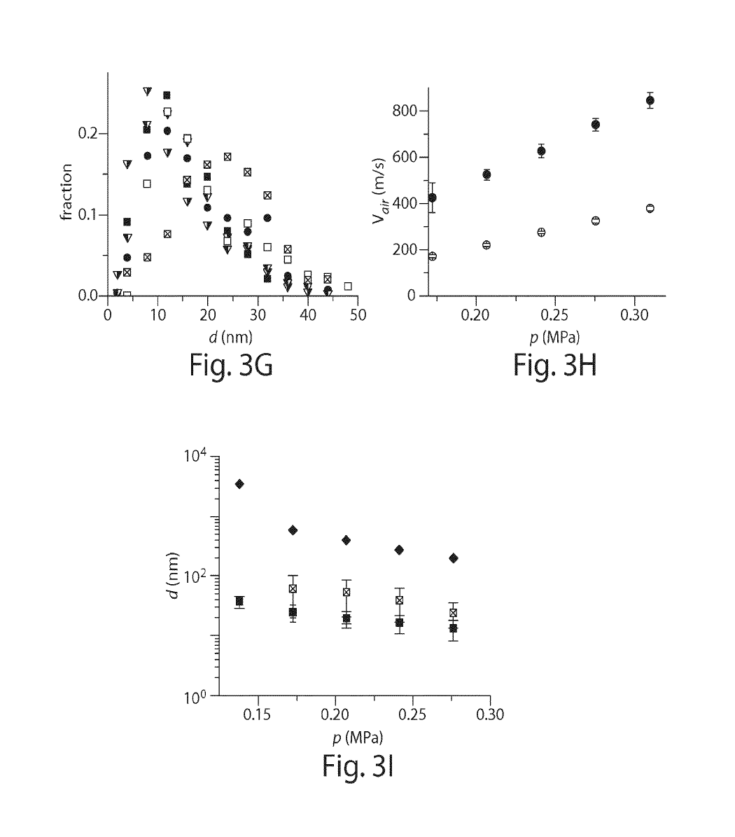

These examples use a microfluidic device made out of poly(dimethyl siloxane) (PDMS); it has inlets for two types of liquids followed by multiple inlets for compressed air. The liquid inlets are merged before they enter the main channel that is divided into five sections defined by the locations of junctions with the air inlets, as shown in FIGS. 3A-C. The liquids are the dispersed phase, the air is the continuous phase. For the device used in this particular example, the junction furthest downstream in the main channel is three-dimensional (3D); it is 300 micrometers tall, whereas all other junctions are two-dimensional (2D) and are 100 micrometers tall, as schematically shown in FIG. 1B. The 3D junction fully surrounds the liquid with air, minimizing the propensity of the liquid drops to contact the channel walls which would lead to their coalescence. The drops exit the nebulator through the nozzle outlet that is formed by slicing the end of the main channel with a razor blade.

To assess the performance of the nebulator, inorganic particles were spray dried. For demonstration, CaCO₃ nanoparticles were produced; this involves an on-chip precipitation reaction initiated by co-injecting two aqueous solutions containing CaCl₂ and Na₂CO₃. These liquids were merged immediately before the solution is spray-dried. The resulting CaCO₃ nanoparticles were collected on a silicon wafer located at a distance of 15 cm from the nebulator outlet. The collected nanoparticles were imaged using scanning electron microscopy (SEM). Alternatively, the particles were collected on a carbon supported transmission electron microscopy (TEM) grid and image them with TEM.

The size of particles produced in conventional spray driers depends on the size of the drops that are formed at their nozzles. A similar correlation was expected for the microfluidic nebulator. To verify this expectation, test different nebulator geometries were tested. Devices with three pairs of air inlets operated only in the jetting regime, where liquid jets were broken into drops at the outlet of the nozzle, similar to the behavior observed in a microfluidic spray drier. Operating in the jetting regime results in partial coalescence of the drops at the nozzle and leads to a broad drop size distribution. This nebulator design produced CaCO₃ particles with sizes ranging from 50 nm to several micrometers. The wide range of particle sizes may be attributed to the broad liquid drop size distribution at the nebulator outlet.

In these examples, control over the size of the drops was achieved by forming droplets on-chip; this can be achieved by operating the nebulator in the dripping regime, which required instability in the liquid jet. To create instability in this example, perturbations to the liquid jet interface must grow without advecting downstream; this was facilitated using a stagnation point of the flow velocity along the interface. The viscous stress in the air was comparable to that in the liquid, v_{liquid} η_{liquid}˜vₐᵢᵣ ηₐᵢᵣ; where v is the velocity and η is the dynamic viscosity.

The liquid velocity at the junction where the liquid first met the air, hereafter called junction 1, was approximately 7 cm/s; thus operating the nebulator in the dripping regime required the velocity of the air in this region of the device to be ˜3 m/s. Consequently, the pressure drop across channel section 1, calculated as the product of the air flow rate and the channel resistance, did not exceed 10 Pa. A pressure of 0.28 MPa was applied to the air inlets; the pressure at the outlet of the nozzle of the device was 0.1 MPa. Therefore, the pressure gradient in the main channel of a device containing only three pairs of air inlets was much larger than 10 Pa and precludes operation of this device in the dripping regime.

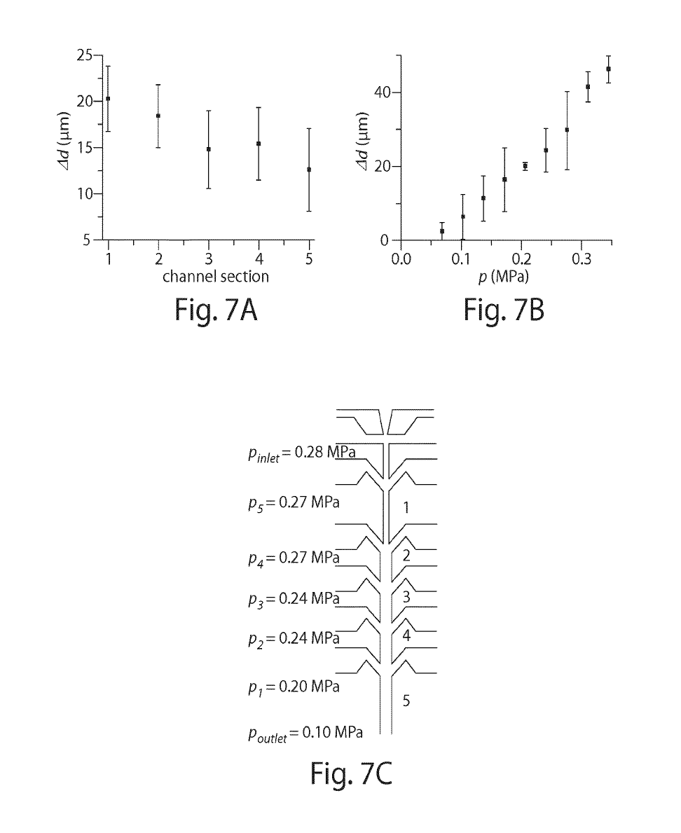

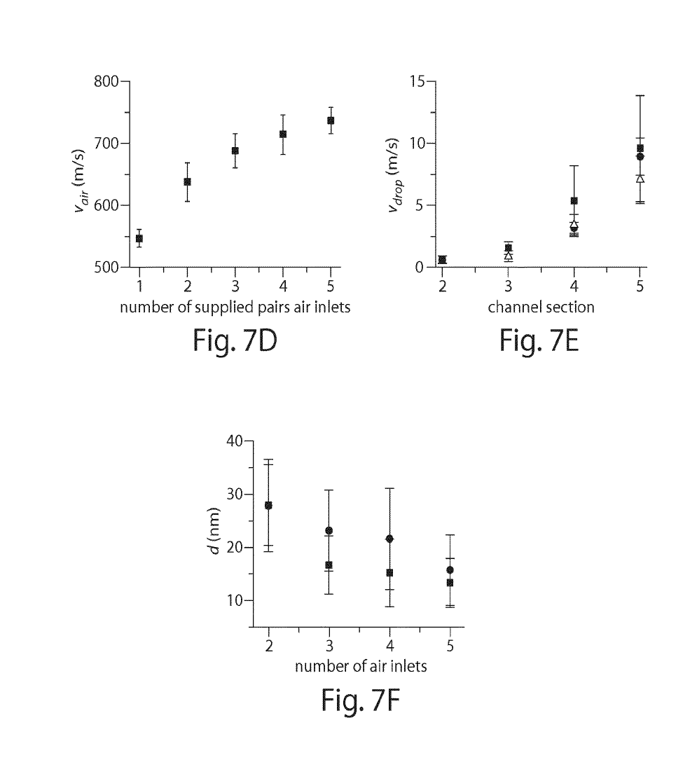

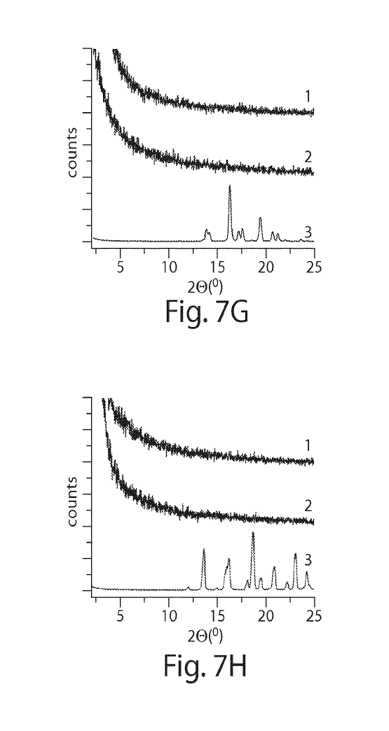

To reduce the pressure gradient in channel section 1, multiple pairs of air inlets were used; this resulted in a more gradual pressure drop along the main channel, as shown in FIGS. 7A-7E. Since the air velocity was proportional to the pressure gradient, it decreased as the pressure gradient between junctions decreased. However, even if the devices possessed as many as six pairs of air inlets, the air velocity in junction 1 was still ten times higher than is required for instability to occur, as shown in FIGS. 3D and 7D. Instead of small droplets, larger plugs were formed and broke up into smaller droplets at the nozzle outlet. Calcium carbonate particles produced with these types of nebulators had a considerably narrower size distribution.

One method of further reducing the nanoparticle size and preventing or reducing aggregation is to improve control over the drop size. This can be achieved by operating the nebulator in the dripping regime whereby an absolute instability is formed in the liquid jet. This required further reduction of the air velocity in junction 1; it could be achieved by designing nebulators that have even more air inlets. In some cases, an absolute instability may form if the interfacial velocity along the direction of the liquid flow vanishes. Thus, it is only the air velocity component along the direction of the main channel that must be small. If the direction of the air inlet is inverted in junction 1, the air flow direction would be opposed to that of the liquid flow. This type of junction has an angle Θ=135°, where Θ (theta) is the angle between the inlet for the liquid and that for the air in junction 1, as shown in FIG. 3B. It resembles the flow focusing geometry in glass capillary devices and forces the air to make a U-turn to enter the main channel. The component of the air velocity vector that is directed parallel to the main channel is therefore slowed down to close to 0 m/s before it is accelerated to the maximal speed the air reached close to the 3D junction. Thus, at some point in the main channel, the air reaches the velocity required to create a stagnation point. Indeed, nebulators with an angle Θ=135° and at least four pairs of air inlets display proper operation in the dripping regime, as shown in FIG. 3C.

Droplets generated in junction 1 were many times larger than those exiting the nebulator outlet. To investigate the reason for this observation, the droplets were monitored as they passed through the main channel using a high-speed camera operated at 38,000 frames per second. Using frame sequences from these movies, the speed of the drops was measured in the different sections of the main channel. In addition, the air velocities were estimated in the different channel sections by measuring the pressure profile in the main channel and the air velocity at the nebulator outlet.

It was found that both the air and droplets strongly accelerate toward the outlet of the device, as shown in FIG. 3D. The air velocity at the 3D junction was supersonic and creates high drag forces on the droplets. This drag force exceeded the surface tension of the drops and thus large droplets sequentially break into many smaller droplets. Consequently, CaCO₃ nanoparticles produced with this type of nebulator were smaller than those produced in devices with an angle Θ=45°. Furthermore, they did not agglomerate, as shown in FIG. 3E.

FIG. 3 shows the microfluidic nebulator used in this example. FIG. 3A shows an overview and FIG. 3B shows a close-up of the microfluidic nebulator; liquids are injected through the darker inlets; air is introduced through the lighter inlets. The angle between the liquid inlet and the first pair of air inlets was an angle Θ. The main channel was divided into sections 1-5, defined by the locations of the different pairs of air inlets. The junction located furthest downstream the main channel was a 300 micrometer tall 3D junction; all the other junctions were two dimensional with a height of 100 micrometers. The scale bar was 100 micrometers. FIG. 3C is an optical micrograph of an operating nebulator with five pairs of 2D air inlets and an angle Θ=135°. Water was used as a dispersed phase, and air as a continuous phase. The arrow indicates the location of a liquid water droplet. The pressure of the air at the inlets was 0.28 MPa, and the flow rate of water was 1 ml/h. The scale bar is 100 micrometers. FIG. 3D shows the evolution of the speeds of the air (circles) and the water drops (diamonds) in the main channel as a function of their location. FIG. 3E is a scanning electron micrograph of spray-dried CaCO₃ nanoparticles. The particles were produced by co-injecting two aqueous solutions containing either 1 mM CaCl₂ or Na₂CO₃. The solutions were combined immediately before droplets were formed. Spray-dried nanoparticles were collected 15 cm away from the outlet.

FIG. 7A shows the pressure-dependent expansion of the different sections of the main channel of a nebulator with five 100 micrometer tall pairs of air inlets. The pressure applied to all air inlets was 0.28 MPa. FIG. 7B shows the expansion of the main channel as a function of the pressure applied to all air inlets; this was a static, equilibrium measurement using nebulators with a sealed outlet. FIG. 7C shows a schematic illustration of the nebulator with the pressure profile for the main channel calculated from the measurements shown in FIGS. 7A and 7 B. The main channel was divided into sections 1-5, defined by the location of the air inlets in this example.

To estimate the air velocity in the main channel of the nebulator, the pressure profile was determined along the channel by measuring its pressure-dependent expansion using confocal microscopy, as shown in FIG. 7A. To convert the expansion of each channel section to pressure, use a calibration curve of the expansion of the main channel as a function of applied pressure in a nebulator that had no outlet was used, as shown in FIG. 7B. The pressure profile was determined in the different main channel sections, as shown in FIG. 7C.

The pressure profile was converted to mean air flow velocity in the different main channel sections using a second calibration curve that relates the velocity of air flow at the three-dimensional (3D) junction of the nebulator as a function of the number of pressurized air inlets. The number of pressurized air inlets was varied without changing the device design by supplying a fixed number of air inlets with air and sealing the remaining inlets. A constant pressure of 0.28 MPa was applied to the inlets supplied with air. A 60 mL gas-tight syringe was connected to the nebulator outlet and the volumetric flow rate was determined by measuring the time required to fill the syringe with 50 cm³ of air. The air velocity was calculated by dividing the volumetric flow rate by the channel cross-sectional area, assuming the pressure at the nebulator outlet was 0.1 MPa. It was found that air velocity increases with the number of inlets supplied with air, as shown in FIG. 7D. Velocity was independent of the location of supplied air inlets within the main channel; friction was neglected in these calculations. Based on these measurements, the air speed was calculated in the main channel. For example, the air speed in section 2, v₂, was calculated using v₂=v_{2 inlets}−v_{1 inlet}, where vᵢₙₗₑₜ and v_{2 inlets} were the air velocities measured if 1 and 2 pairs of inlets are supplied with air; these values are summarized in Table 1. The air speed in the other channel sections was calculated similarly, the results are summarized in Table 1.

TABLE 1

| channel | νₐᵢᵣ | V_{drop} |
|---|---|---|
| section | (m/s) | (m/s) |
| 1 | 23 | 0.6 |
| 2 | 26 | 1.6 |
| 3 | 50 | 5.3 |
| 4 | 26 | 1.6 |
| 5 | 740 | |

### Example 3

If the nanoparticle size was determined by the droplet size, and the droplet size is determined by the balance of the surface tension and drag forces, then this nebulator should be generally applicable to any system, including aqueous and non-aqueous systems. To test if the nebulator could also produce organic nanoparticles from organic solutions, 5 mg/ml of fenofibrate, a poorly water-soluble drug, was dissolved in ethanol. This solution was spray-dried by applying 0.28 MPa to the air inlets and the dried nanoparticles were collected on a silicon substrate that is located 8 cm away from the nebulator outlet. The same nebulator design allowed the production of fenofibrate nanoparticles with sizes below 20 nm, as shown in FIG. 3F. This was more than 10 times smaller than the smallest particles produced with commercially available spray driers.

It was expected that the size of spray-dried nanoparticles would depend on the droplet size and the initial solute concentration but not on the chemical composition of the solute. To probe this expectation, clotrimazole, danazol, and estradiol, all poorly water-soluble drugs, were dissolved in ethanol. The solute concentration was kept constant at 5 mg/ml. It was found the size of the resulting spray-dried drug particles was essentially identical, as shown in FIG. 3G.

Nanoparticle size was expected to increase as the cube root of the solute concentration. To test this idea, the solute concentration was increased. However, if the drug concentration exceeded 10% of its saturation concentration, drugs started to crystallize in the main channel; these crystals adsorb on the main channel walls and clogged the device. By contrast, a fivefold increase of the CaCl₂ and Na₂CO₃ did not compromising the operation of the nebulator.

The size of the resulting spray-dried CaCO₃ nanoparticles increased by a factor of

$$\sqrt[x]{5} \approx 1.7 ,$$

as shown in FIG. 3G. The nanoparticle size should also increase with increasing drop size. To test this idea, the droplet size was varied by changing the drag force exerted on droplets and keeping the surface tension constant. The drag force was proportional to the velocity of the air squared. Therefore, the droplet and consequently the nanoparticle size was expected to decrease with increasing air velocity. To test this expectation, the air velocity was varied by applying different pressures to the air inlets. It was found that the size of spray-dried CaCO₃ and fenofibrate nanoparticles decreased with increasing pressure applied to the air inlets, as shown in FIGS. 3H and 3I. Thus, the size of spray-dried particles could be controlled by controlling the pressure applied to the air inlets.

FIG. 3F shows a scanning electron micrograph of spray-dried fenofibrate nanoparticles produced from an ethanol-based solution containing 5 mg/ml of fenofibrate. The drug/ethanol solution was spray-dried using the same flow parameters as for the production of CaCO₃ nanoparticles. Spray-dried fenofibrate nanoparticles were collected at a distance of 10 cm from the device outlet. The scale bar is 200 nm. FIG. 3G shows the size distribution of spray dried fenofibrate (closed boxes), clotrimazole (circles), danazol (half-filled triangles, left half filled), estradiol (half-filled triangles, right half filled), and CaCO₃ nanoparticles produced from an solutions containing initially 10 mM (open squares) and 50 mM salts (boxes with x's). FIG. 3H shows the velocity of air at the 3D junction (open circles) and at the outlet of the nebulator (filled circles) as a function of the pressure applied to the air inlets. FIG. 3I shows the size of liquid drops (filled diamonds), spray-dried fenofibrate (filled squares), and CaCO₃ nanoparticles produced from 5 mM salt solutions (boxes with x's), as a function of the pressure applied to the air inlets.

FIG. 7F shows the influence of the device geometry on the size of spray-dried fenofibrate nanoparticles. Fenofibrate nanoparticles were spray-dried with nebulators containing two to five pairs of 100 micrometer tall air inlets and Θ=135° (filled squares) and Θ=45° (filled circles). Fenofibrate was dissolved in ethanol at 5 mg/ml. 0.28 MPa was applied to the air inlets and the ethanol-fenofibrate solution was injected at a rate of 1 ml/h. The size of the resulting spray dried fenofibrate nanoparticles from SEM images was measured.

To investigate the influence of the design of the nebulator on the size of spray-dried fenofibrate nanoparticles, fenofibrate was dissolved in ethanol at a concentration of 5 mg/ml and nanoparticles were created using nebulators comprised of two to five pairs of 100 micrometer tall air inlets and Θ=135° and Θ=45°. The size of fenofibrate nanoparticles decreased with increasing number of air inlets; this can be attributed to increasing air velocity at the 3D junction of the device. This resulted in higher drag forces and consequently smaller droplets. Interestingly, fenofibrate nanoparticles produced in devices with Θ=135° were consistently smaller than those produced in devices with Θ=45°, in analogy to spray dried CaCO₃ nanoparticles, as shown in FIG. 7F.

### Example 4

The size of inorganic nanoparticles can influence their structure. To assess if this is also true for organic nanoparticles, this example investigates the structure of fenofibrate nanoparticles as a function of their size using differential scanning calorimetry (DSC). Fenofibrate has a melting point Tₘ of 80° C. Thus, for the crystals, the endothermic melting peak can be expected to be at 80° C.

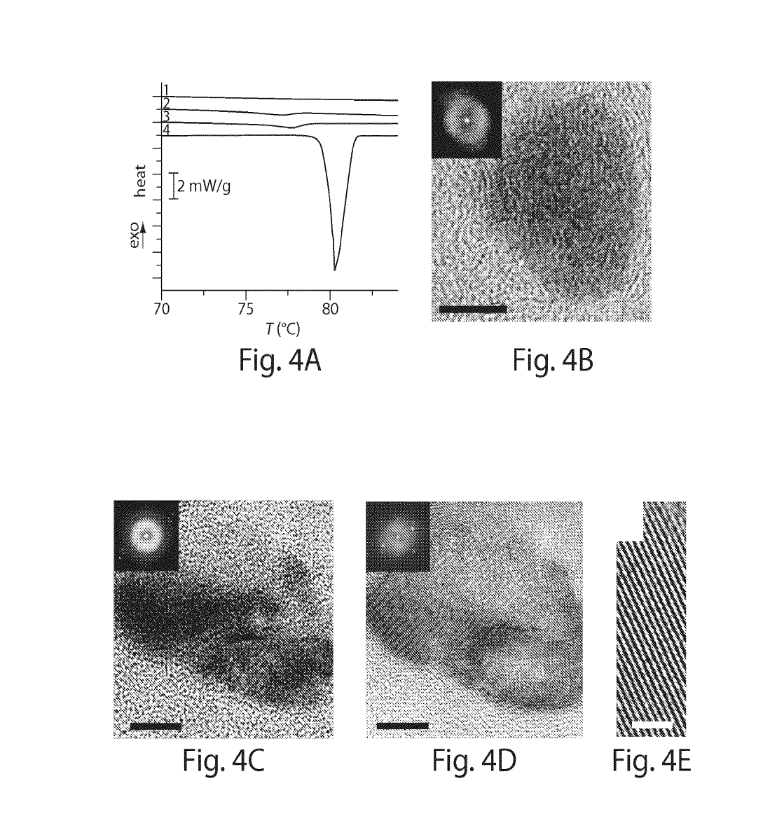

However, surprisingly, for spray-dried fenofibrate nanoparticles below 20 nm, no melting peak was observed. By contrast, a small melting peak was present for particles larger than 20 nm. Integrating the area of the melting peak revealed that 3 vol % of particles with a diameter of 20 nm, and 6 vol % of 25 nm large particles is crystalline, as shown in FIG. 4A. The depression of Tₘ from 80.3° C. for bulk fenofibrate to 77.7° C. for 25 nm and to 77.3° C. for 20 nm nanoparticles was believed to be due to their small sizes.

The absence of a melting peak for particles smaller than 20 nm suggests that these particles were amorphous. To further explore this finding, high resolution TEM was performed on of spray-dried fenofibrate particles. In agreement with the DSC results, there was no sign of crystallinity for nanoparticles smaller than 15 nm, as shown in FIG. 4B. By contrast, nanoparticles larger than 25 nm contained many small crystals, as shown in the Fourier transformation in FIG. 4C. However, even particles as large as 30 nm were not entirely crystalline; instead, small crystals were embedded in an amorphous matrix. The amorphous matrix crystallized into a single crystal if heated with an electron beam. By contrast, the size of the crystalline nuclei did not change precipitously if exposed to the same amount of heat, as shown in FIGS. 4C-4E. Thus, the resulting particles included a mixture of the crystalline nuclei formed during the spray-dry process, and the crystallized matrix; they were therefore polycrystalline, as shown in the Fourier transformation of FIG. 4D.

To understand the absence of crystalline structures in nanoparticles smaller than 15 nm, along with the co-existence of amorphous and crystalline phases for larger particles, the mechanism by which nanoparticles form in droplets was examined. Nanoparticles usually form by nucleation and growth; the nuclei, and therefore the resulting particles, can be crystalline or amorphous. However, if droplets evaporate too quickly for nuclei to form, solute molecules may cluster together by the surface tension force of the evaporating droplet; the resulting nanoparticles are then expected to be amorphous. To determine which mechanism dominates particle formation, the cumulative number of nucleation events in a droplet was estimated as it evaporates; this depends on the time available for nucleation and therefore the solvent evaporation rate, and the initial droplet size.

Nucleation becomes possible if the solute concentration exceeds the saturation concentration. The solute concentration in a droplet steadily increased as the solvent evaporates, and eventually reaches the saturation concentration; thereafter, nuclei can form. Nucleation ceases when all the solvent is evaporated; the final droplet size may be taken to be equal to that of the resulting nanoparticle. Thus, the time available for nuclei to form depended on the solute saturation concentration and the solvent evaporation rate.

To estimate the time during which fenofibrate nuclei can form in ethanol based drops, the saturation concentration of crystalline fenofibrate was determined in ethanol at 20° C. to be 50 mg/ml. The amorphous phase was calculated using the regular solution model, as detailed below; it was 13 times higher than that of the crystalline phase. Consequently, the time available for nucleation was much longer for the crystalline than for the amorphous phase.

To quantify the nucleation time, the time required to evaporate a drop, tₑᵥₐₚ, was calculated as a function of its initial size, using the impingement law from the kinetic theory, as detailed below. The supersonic speed in the last part of the nebulator prevented saturation of the air with solvent as it quickly transported the gaseous solvent molecules to the nebulator outlet; it can be assumed that the partial pressure of the solvent in the air was negligible. The initial droplet size was estimated from the known initial solute concentration and the size of spray-dried nanoparticles, assuming that only one nanoparticle forms per droplet. Irrespective of the mechanism by which nanoparticles form, exactly one nanoparticle per droplet was expected; even if multiple nuclei form within a single droplet, these nuclei are pulled together by the surface tension force of the evaporating droplet, resulting in a single agglomerate. It was found that nanoparticles with a diameter of 14 nm were produced in ethanol drops with ˜85 nm diameter. For example, in droplets of this size, initially containing 5 mg/ml fenofibrate, crystalline nuclei can form for 1.6 microseconds, while amorphous ones form for only 0.2 microseconds. By contrast, 40 nm nanoparticles formed in ethanol drops with a diameter of 250 nm, crystalline nuclei can then form for 4.4 microseconds, amorphous ones for 0.6 microseconds.

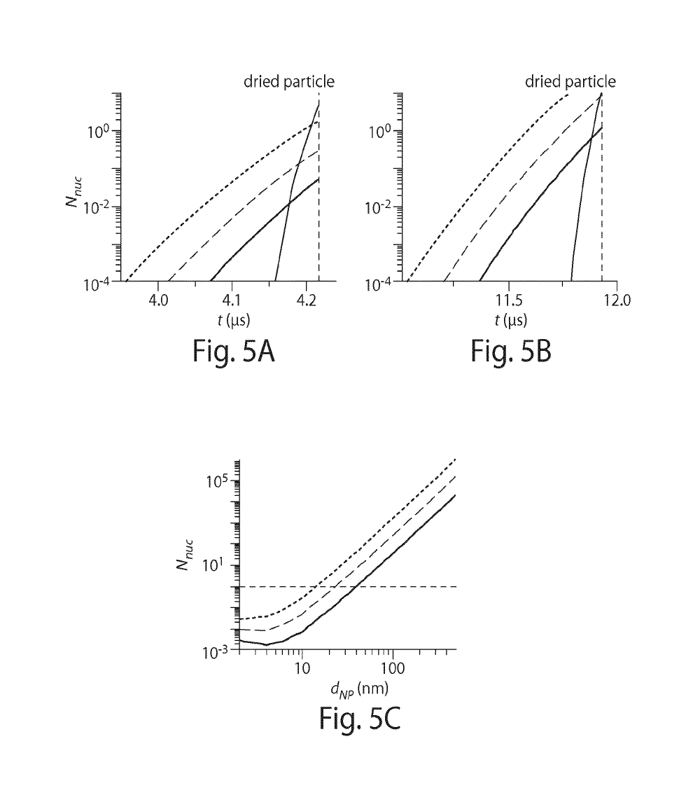

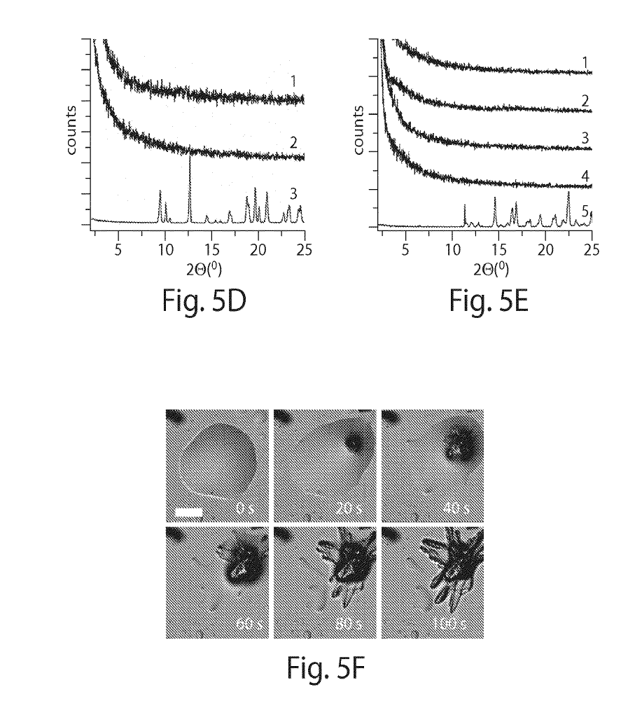

To estimate the probability for one nucleus to form as a droplet evaporates, the cumulative number of nucleation events N_{nuc} in a single droplet was calculated, as detailed below. The only unknown parameters in this calculation were the interfacial energies (gamma) between the solution and the nucleation phase for the amorphous nucleus γₐₘₒᵣₚₕ (gamma-amorph) or the crystalline one γ_{cryst} (gamma-cryst). The DSC and TEM data allow an estimate of a lower and upper limit of γ_{cryst} (gamma-cryst). Nanoparticles with sizes below 15 nm were amorphous; this implied that no crystalline nuclei forms during the evaporation process, indicating γ_{cryst} (gamma-cryst)>12.1 mJ/m². By contrast, nanoparticles larger than 40 nm were crystalline; consequently, at least one crystalline nucleus forms during the evaporation of 250 nm droplets. This implies that γ_{cryst} (gamma-cryst)<13.7 mJ/m². Indeed, these values were similar to those of the crystal melt interfacial energies of alkanes of similar molecular weights. If amorphous particles with diameters of 14 nm grow from amorphous nuclei, at least one amorphous nucleus forms in 85 nm droplets; this requires γₐₘₒᵣₚₕ (gamma-amorph)<4.1 mJ/m². That the interfacial energy for the amorphous phase is lower than that for the crystal can be attributed to the lower entropy loss associated with the localization of the solvent molecules near a disordered surface compared to a periodic crystalline one. Based on these values, calculate N_{nuc} was calculated for droplets with diameters of 85 nm and 250 nm, as shown in FIGS. 5A-5B.

The extremely short time available for nucleation combined with the low probability for nuclei to form in 85 nm drops indicated that the nanoparticles form during the final stage of droplet evaporation: fenofibrate molecules are then clustered together by the surface tension forces. This mechanism is further supported by the co-existence of amorphous and crystalline phases in particles with diameters between 30 and 40 nm. Particles of these sizes are produced in droplets with diameters between 180-250 nm; the time available for nuclei to form was more than twice that in 85 nm drops, strongly increasing the probability for crystalline nuclei to form. These larger nanoparticles often had multiple crystalline domains that are separated by an amorphous matrix, as shown in FIG. 4C; this indicated that multiple crystalline nuclei form during evaporation of these droplets. However, these nuclei cannot grow to consume all the solute; instead, the remaining solute molecules clustered together by surface tension and formed an amorphous matrix that crystallized into a single crystal upon heating, as shown in FIGS. 4D-4E. The percentage of the amorphous matrix decreased with increasing nanoparticle size, as shown in FIG. 4A. The time available for nucleation and growth of a crystal increases with increasing droplet size and hence nanoparticle size; the fraction of crystals was therefore higher in larger particles.

To determine the characteristic size below which fenofibrate nanoparticles are entirely amorphous, the cumulative nucleation events in a droplet as a function of its size were calculated. Assuming exactly one particle per droplet forms, the characteristic droplet size below which N_{nuc} of the crystalline phase can be determined to be less than 1 into a characteristic nanoparticle size below which they are amorphous. It was found that N_{nuc} of the crystalline phase was less than one for droplets that are smaller than 85 nm; consequently, nanoparticles smaller than 15 nm may be amorphous, as shown in FIG. 5C.

While the characteristic size below which particles were entirely amorphous is system-specific, the suppression of nucleation in small droplets relies on a generally applicable physical principle. Hence, it was expected that other drugs formulated with the nebulator under identical conditions may be amorphous as well. To test this expectation, the structure of spray-dried clotrimazole, estradiol, and danazol was examined using X-ray diffraction (XRD). To keep the nucleation time the same, they were dissolved at 10% of their saturation concentration. In agreement with the above, these spray-dried drug nanoparticles were amorphous.

Production of amorphous drugs may require the addition of excipients. However, despite the presence of excipients, these drugs tend to crystallize with time leading to a change in their dissolution kinetics; this prevents their application in industry. Strikingly, clotrimazole, estradiol, and danazol did not crystallize if stored under ambient conditions for at least four weeks, as shown in FIGS. 5D, 7G, and 7H. These drugs all had glass transition temperatures, T_{g}, above room temperature; they were therefore glasses at room temperature. By contrast, T_{g} of fenofibrate is =−20° C.; thus, it was a metastable undercooled liquid at room temperature. Consequently, fenofibrate crystallizes within a few weeks if stored under ambient conditions, as shown in FIG. 5E. Once nucleated, the crystal grows rapidly into the liquid phase, as shown in FIG. 5F. This suggests that nucleation is a rate determining step in the crystallization of this system.

FIG. 4A shows the morphology of spray-dried fenofibrate nanoparticles. FIG. 4A shows differential scanning calorimetry (DSC) spectra of fenofibrate nanoparticles spray-dried by applying 1) 0.28 MPa, 2) 0.21 MPa, and 3) 0.17 MPa to the air inlets are compared to the 4) reference spectrum of bulk fenofibrate. FIG. 4B shows high resolution TEM micrograph of fenofibrate spray-dried by applying 0.28 MPa to the air inlets with a Fourier transform in the inset. The scale bar is 5 nm. FIGS. 4C-4D show high resolution TEM images of fenofibrate spray-dried by applying 0.17 MPa to the air inlets. FIG. 4C shows that fenofibrate particles were only partially crystalline and FIG. 4D shows fully crystallized particles if irradiated with an electron beam for more than 30 s. The scale bar is 10 nm. FIG. 4F shows that the amorphous phase transformed into a single crystal with a characteristic lattice plane spacing of 3.6 \acute{Å} (0.36 nm) perpendicular to the electron beam. The scale bar is 2 nm.

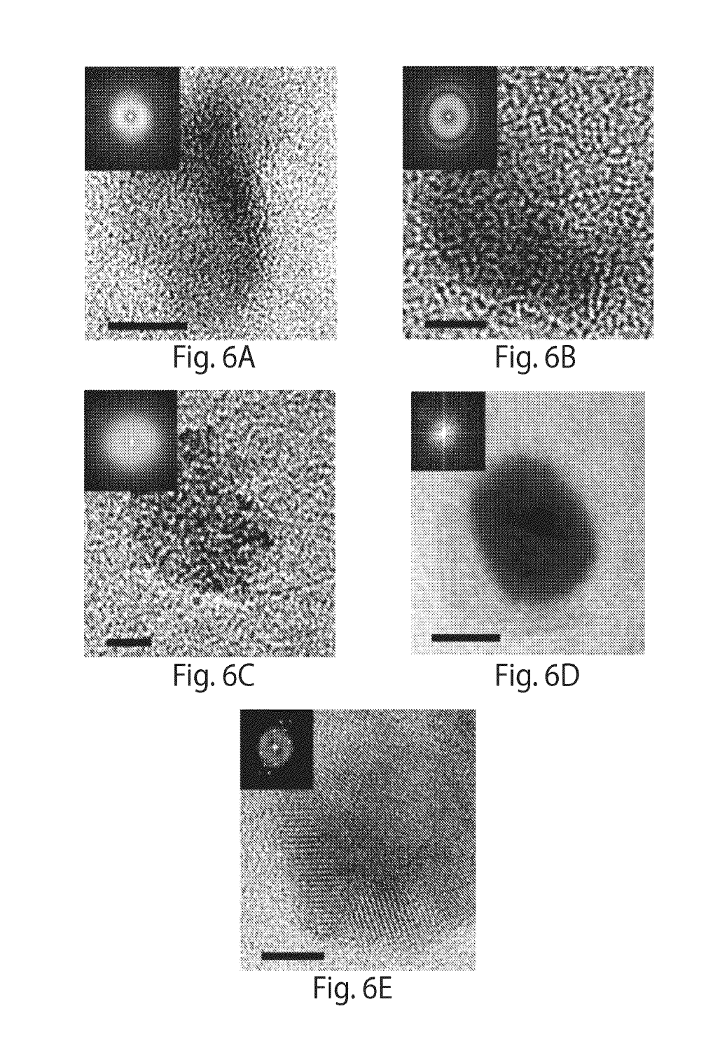

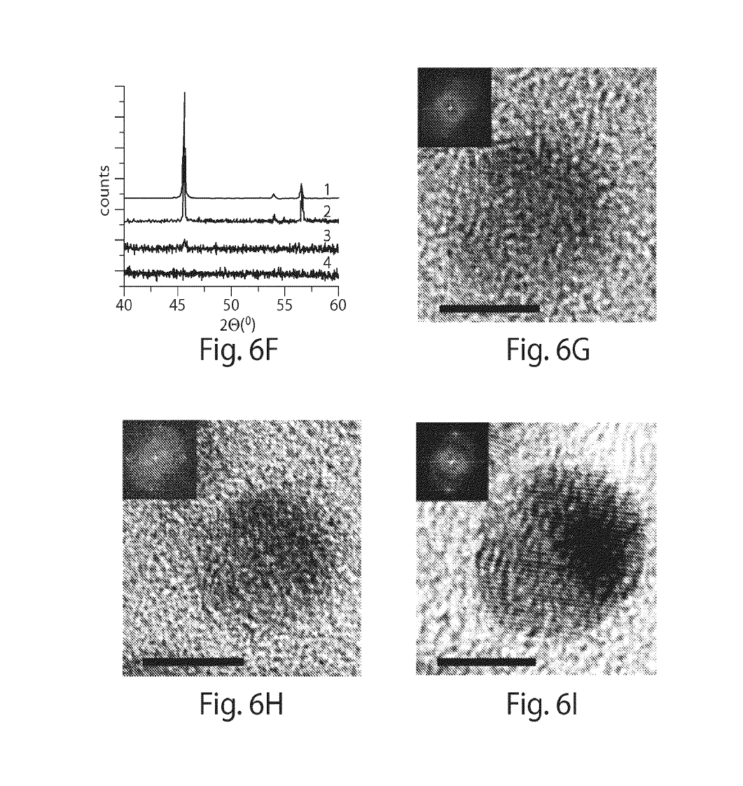

FIG. 6 shows examples of nucleation and crystal growth in droplets. The cumulative number of nucleation events N_{nuc} in a (FIG. 6A) 85 nm and (FIG. 6B) 250 nm diameter droplet calculated as a function of the time during its evaporation is shown in these figures. The results are shown for the amorphous phase with interfacial energy γₐₘₒᵣₚₕ (gamma-amorph)=4.1 mJ/m² (steep solid line), and for the crystalline phase with γ_{cryst} (gamma-cryst)=12.1 mJ/m² (dotted line), γ_{cryst} (gamma-cryst)=12.7 mJ/m² (dashed line), and γ_{cryst} (gamma-cryst)=13.3 mJ/m² (solid line). The vertical dashed line indicates the time when solvent evaporation was complete. Droplets with a diameter of 85 nm produce 14 nm fenofibrate nanoparticles, those with a diameter of 250 nm yield 40 nm nanoparticles. FIG. 5C shows the cumulative number of nucleation events in an ethanol drop initially containing 5 mg/ml fenofibrate calculated as a function of the size of the resulting fenofibrate nanoparticles. FIG. 5D shows X-ray diffraction spectra of clotrimazol spray-dried by applying a pressure of 0.28 MPa to the air inlets 1) immediately after the sample is produced, and 2) after storing it for 4 weeks under ambient conditions; 3) reference spectrum of bulk clotrimazol. FIG. 5E shows X-ray diffraction spectra of fenofibrate spray-dried by applying a pressure of 0.28 MPa to the air inlets 1) immediately after the sample is collected, after storing it at 2) 25° C. for 4 weeks, 3) 40° C. for 4 weeks, and 4) 60° C. for 3 d; 5) reference spectrum of bulk fenofibrate. FIG. 5F shows an optical time lapse micrograph of the crystallization of amorphous fenofibrate. The sample was spray-dried by applying 0.28 MPa to the air inlets. It was then heated to 50° C. where its crystallization was initiated with crystalline fenofibrate seeds. The scale bar is 50 micrometers.

FIGS. 7G-7H show the stability of amorphous drugs at room temperature. FIG. 7G shows danazol and FIG. 7H shows estradiol spray dried by applying a pressure of 0.28 MPa to the air inlets. X-ray diffraction spectra were acquired 1) directly after samples are prepared, and 2) after storing them for 2 weeks at 25° C. 3) Reference spectra of bulk drugs.

Spray-dried amorphous drugs with a T_{g} above room temperature did not crystallize if stored under ambient conditions for at least 2 weeks, as shown in FIGS. 7G-7H. By contrast, amorphous drugs that were stabilized with excipients typically crystallized over time. This indicates that drugs stabilized with excipients crystallized through heterogeneous nucleation.

### Example 5

It was expected that fast evaporation of sufficiently small droplets not only suppressed nucleation in ethanol-based drops, but also in droplets of other solvents. This suggests that different types of amorphous organic and inorganic nanoparticles can be produced. To probe this expectation, in this example, the structure of CaCO₃ nanoparticles, spray-dried from aqueous solutions, was examined using high resolution TEM. The nebulator operated at an air pressure of 0.28 MPa produces 120 nm water drops; these drops evaporate within 27 microseconds. By contrast to the drugs, these inorganic nanoparticles formed through a chemical reaction; the solution became supersaturated upon combining the two liquids, immediately before droplets were formed. Therefore, the time available for nuclei to form was more than 10 times longer than that for drug nuclei in ethanol drops. To prevent nucleation of CaCO₃ crystals before droplets were formed, aqueous solutions were injected containing maximally 5 mM of salts. Despite the longer time for nuclei to form, no sign of crystallinity was observed in these CaCO₃ nanoparticles, as shown in FIG. 6A. By contrast to the currently known amorphous CaCO₃ nanoparticles that require appropriate organic surfactants to prevent crystallization, these amorphous nanoparticles formed in the absence of any organic additives.

To test if amorphous inorganic nanoparticles could be produced from materials whose amorphous phase is less well known than that of CaCO₃, BaSO₄ nanoparticles were formed through an on-chip aqueous precipitation reaction and iron oxide nanoparticles by increasing the pH of an aqueous solution containing a mixture of FeCl₂ and FeCl₃. Remarkably, none of these nanoparticles revealed any sign of crystallinity, as shown in FIGS. 6B-6C. This indicated that the formation of crystalline nuclei is also suppressed in these systems.

To examine if nucleation of materials that have a very strong tendency to crystallize could be suppressed, NaCl nanoparticles were spray-dried from aqueous solutions and characterized with high resolution TEM and XRD. Nucleation could only be suppressed if the nucleation time is very short. To test this expectation, two types of aqueous solutions initially containing 40 mM and 400 mM NaCl were spray-dried. To calculate the time available for crystalline nuclei to form, the saturation concentration of NaCl was measured in water at room temperature to be 6.3 mol/l. The nebulator in this example operated at an air pressure of 0.28 MPa produces 120 nm sized water drops. In these droplets, crystalline NaCl nuclei can form for 2.4 microseconds if the initial NaCl concentration was 40 mM, and for 5.1 microseconds if the initial NaCl concentration is 400 mM. Strikingly, NaCl nanoparticles spray-dried from solutions containing 40 mM NaCl showed no sign of crystallinity, as indicated in FIG. 6D. By contrast, nanoparticles produced from solutions initially containing 400 mM NaCl were polycrystalline, as shown in FIG. 6E. These results were supported by XRD measurements where no diffraction peaks were seen if NaCl nanoparticles are spray-dried from solutions initially containing 40 mM salt. By contrast, the (220) reflection of the NaCl structure at 2θ=45.5° was seen if an equal mass of NaCl nanoparticles produced from a solution containing 400 mM NaCl was examined, as shown in FIG. 6F. This suggested that nucleation can be suppressed if the nucleation time is sufficiently short, even for materials that have a strong propensity to crystallize, such as NaCl.

To test if NaCl nanoparticles lacking long-range periodic order possess small crystals that cannot be resolved with HRTEM, they were exposed to an electron beam to locally increase the temperature; this increases the probability for nuclei to form and accelerates the crystal growth. Surprisingly, these nanoparticles crystallized into a single domain if exposed to an electron beam, as shown in FIG. 6G-6I. If multiple small crystals were present, they would be expected to grow simultaneously and result in a polycrystalline nanoparticle. The transformation of a disordered nanoparticle into a single crystal thus suggested that it was initially amorphous. These results demonstrate the potential of the microfluidic nebulator to suppress nucleation in all systems. The nebulator was therefore a powerful, generally applicable tool to produce amorphous organic and inorganic nanoparticles.

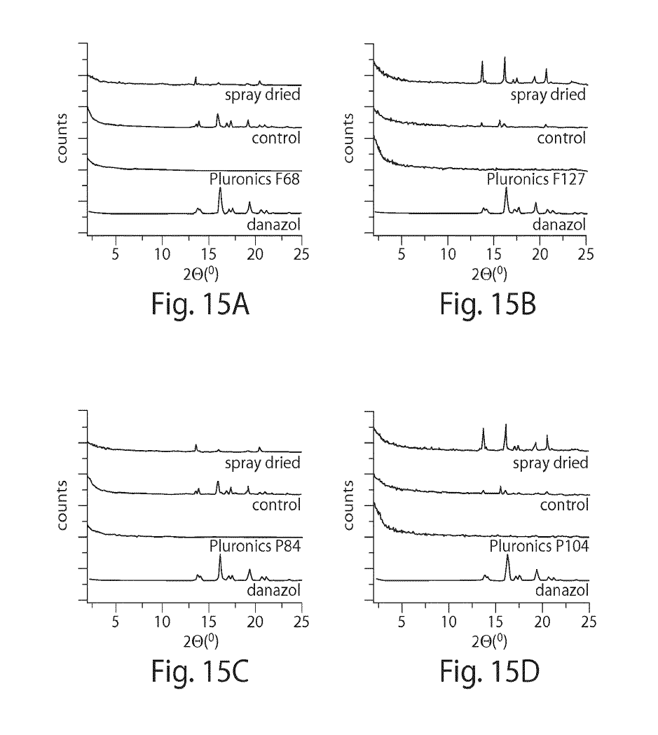

FIG. 6 shows examples of the morphology of spray-dried inorganic nanoparticles, including high resolution TEM images, with Fourier transform inlets, of (FIG. 6A) CaCO₃, (FIG. 6B) BaSO₄, and (FIG. 6C) iron oxide nanoparticles produced in the nebulator in this example. The pressure at the air inlets was 0.28 MPa, and the total flow rate of the aqueous phases was 1 ml/h. CaCO₃ and BaSO₄ nanoparticles were produced through a precipitation reaction, iron oxide nanoparticles by increasing the pH of an aqueous solution containing FeCl₂ and FeCl₃. The scale bars are 10 nm. FIG. 6D shows a high resolution TEM image of NaCl nanoparticles spray-dried from an aqueous solution initially containing 40 mM NaCl. The scale bar is 5 nm. FIG. 6E shows a high resolution TEM micrograph of a NaCl nanoparticle spray-dried from an aqueous solution initially containing 400 mM NaCl. The scale bar is 5 nm. FIG. 6F shows an X-ray diffraction spectrum of 1) reference NaCl, 2) crystalline NaCl produced by slowly evaporating an aqueous solution containing 40 mM NaCl, spray-dried NaCl nanoparticles produced from an aqueous solution containing 3) 400 mM and 4) 40 mM NaCl. The pressure applied to the air inlets was 0.28 MPa. FIG. 15G-15I shows a time series of high resolution TEM images of NaCl nanoparticles spray-dried from an aqueous solution containing 40 mM NaCl (FIG. 15G) with minimal and (FIGS. 15H-15I) increasing exposure to the electron beam. The scale bars are 5 nm.

To demonstrate feasibility to parallelize microfluidic nebulators, three adjacent nebulators were simultaneously operated; this was achieved by delivering liquids and air through 500 micrometer tall by 1.5 mm wide distribution channels to the individual nebulators. The parallelization strategy employed here is not limited to just three, but can be extended to parallelize many units. Thus, the possibility to produce additive-free, amorphous inorganic and organic nanoparticles with unprecedented small sizes in a continuous process renders the nebulator an attractive, versatile tool for many different scientific and industrial applications.

### Example 6

This example illustrates calculations of nucleation events, in accordance with certain embodiments of the invention. To investigate the mechanism by which nanoparticles form, the homogeneous nucleation frequency was calculated, which is the number of events per unit volume and unit time, J=J₀e^{-w*/k}^{_{B}}^{T}, for the amorphous and crystalline phase; w* is the work of forming the nucleus, k_{B} is the Boltzmann constant and T is the temperate. The pre-factor was approximated as J₀=cλ²/D, where c is the concentration of fenofibrate in the drop, λ is the average distance between two solute molecules and D is the diffusion coefficient of fenofibrate in ethanol. D was taken to be 2.38×10⁻⁶ cm²/s, a value measured for fenofibrate dispersed in a mixture of water and methanol.

The work of formation of an isotropic, spherical nucleus is defined as

$${w}^{*} = \frac{16 π}{3} \frac{{γ}^{s}}{{( Δμρ )}^{z}} ,$$

where γ is the interfacial energy associated with the nucleus-solution interface, Δμ is the chemical potential, and ρ is the density of the nucleating phase. The density of fenofibrate is 1.18 g/cm³. Δμ=ε(1−x_{squ})²+RT ln(x_{equ}), where ε is the interaction parameter, x_{equ} the equilibrium mole fraction, and R is the gas constant. The equilibrium mole fraction is defined as

$${x}_{equ} = \frac{1}{1 - \frac{{V}_{solute}^{0}}{{V}_{solvent}^{0}} + \frac{1}{{V}_{solvent}^{0} {c}_{solute , equ}}} ,$$

where V⁰ is the molar volume of the solute and solvent and c_{solute,equ} is the solute concentration in equilibrium with its bulk. It was assumed that Vₛₒₗᵤₜₑ⁰=Vₛₒₗᵥₑₙₜ⁰=0.05838 l/mol; thus, x_{equ}=Vₛₒₗᵥₑₙₜ⁰c_{solvent,equ}. c_{equ} was experimentally determined to be 0.14 mol/l for crystalline fenofibrate in ethanol. C_{equ, amorph} was estimated using a regular solution model. For this, the heat of melting ΔH_{f} of fenofibrate was experimentally determine to be 28.88 kJ/mol using DSC. The difference between the chemical potential of the amorphous and crystalline phase at

$$T = {T}_{rn} - Δ T is {μ}_{amorph}^{0} - {μ}_{cryst}^{0} = \frac{Δ {H}_{f} Δ T}{{T}_{m}} ,$$

where Tₘ is the melting point of fenofibrate. The chemical potential of the solute is modeled by a regular solution model with a single interaction parameter, ε, and with the amorphous phase as the pure state; μₛₒₗᵤₜₑ=μₐₘₒᵣₚₕ⁰+ε(1−x)²+Rt ln(x), where x is the mole fraction of the solute. The interaction parameter was obtained from the equilibrium condition with the crystal μ_{cyst}⁰=μₐₘₒᵣₚₕ⁰+ε(1−x_{cryst,equ})²+RT ln(x_{cryst,equ}), since X_{cryst, equ} is known, this gives ε=6.699 J/mol. The equilibrium concentration against the amorphous phase was obtained from μₐₘₒᵣₚₕ⁰=μₐₘₒᵣₚₕ⁰+ε(1−x_{amorph,equ})²+RT ln(x_{amorph,equ}), which gives x_{amorph, equ}=0.105 or c_{amorph, equ}=1.80 mol/l. Thus, c_{amorph,equ} is 13 times higher than c_{cryst, equ}.

To determine c and λ as a function of time, the evaporation time of the droplet was calculated using the impingement law from the kinetic theory. The number of molecules that impinge a surface per time and surface area is

$$Z = \frac{{p}_{equ}}{\sqrt{2 π m {k}_{B} T}} ,$$

where p_{equ} is the vapor pressure of the solvent at room temperature, and m the mass of a solvent molecule. The time required to decrease the radius of the droplet by the size of one molecule as

$$Δ t = \frac{1}{{zA}_{molecule}} ,$$

where A_{molecule} is the area of a solvent molecule. By iterating this calculation until the radius of the drop equals that of the dry nanoparticle, the volume of the droplet as a function of the evaporation time could be calculated.

The total number of molecules per droplet was calculated using the known initial concentration of solutes and the initial drop size. The number of solute molecules remained unchanged during drop evaporation; this enables the calculation of the solute concentration as the drop evaporates by dividing the number of molecules per droplet by the droplet volume at each time step. Similarly, the intermolecular distance λ and J₀ was calculated as the drop evaporates. The only unknown parameters required to calculate J are γ_{cryst} and γₐₘₒᵣₚₕ. Therefore, the cumulative number of nucleation events was calculated in a drop N, defined as N=Σᵢ Jᵢ ΔtV_{drop,i}, for different values of γ, as described above.

### Example 7

This example illustrates various materials and methods used in the above examples.

Materials. Na₂CO₃, FeCl₂, FeCl₃, BaCl₂, K₂SO₄, NaOH, trichlorododecylsilane, and polyethylene glycol mono-acrylate (PEGMA) were obtained from Sigma Aldrich, ethanol from VWR, CaCl₂ from Mallinckrodt Baker, Sylgard 184 PDMS from Dow Corning, and SU-8 2100 from MicroChem Corp. Fenofibrate, clotrimazole, danazol and estradiol are obtained from BASF.

Device fabrication. The 2D and 3D microfluidic nebulators were fabricated using soft lithography. Briefly, masks were designed using AutoCAD and printed with a resolution of 20000 dpi. Single-side polished Si wafers (University Wafer) were spin coated with SU-8 2100 photoresist at 3000 rpm for 30 seconds. The photoresist was pre-baked at 95° C. before the pattern of the mask was transferred to the SU-8 2100 through UV illumination (OAI Model 150). The photoresist was post baked and developed using PGMEA, resulting in master molds. Subsequently, PDMS replicas were made: the base and crosslinker were mixed at a mass ratio of 10 to 1, poured into the master mold and baked at 65° C. for at least 24 h. Certain two dimensional nebulators had maximally five pairs of air inlets and did not contain the last 3D air inlet junction. To simplify the alignment of the top and bottom parts of the 3D devices, complementary features were introduced into the top and bottom halves of the PDMS devices; they serve as lock and key structures and facilitate alignment. The top and bottom halves of the PDMS devices were bonded using an O₂ plasma (Gala Instruments). After bonding, the microfluidic channel walls were rendered fluorophilic by incubating the channels with a perfluorinated oil solution containing 1 vol % perfluorinated trichlorosilanes 10 min, subsequently rinsing them with perfluorinated oil and drying them with compressed air. The outlet of the nebulator was formed by slicing through the nozzle channel with a razor blade.

Parallelized nebulators were fabricated identically to the single devices. The distance between adjacent 3D nebulators was 8 mm. The liquid and air inlets of the parallelized nebulators were connected on a second layer via 500 micrometer tall distribution channels. Distribution channels were bonded to the parallelized devices using O₂ plasma in analogy to the bonding of the top and bottom part of the nebulators.

Device operation. The pressure at the air inlets was set to 0.28 MPa (40 psi). Liquids and the air were connected to the microfluidic device using polyethylene tubing with an inner diameter of 0.33 mm (Scientific Commodities Inc.). The operation of the microfluidic nebulator was monitored using a high-speed camera (Phantom V7.3) operated at a frame rate of 38000 fps.

Calcium carbonate nanoparticles were synthesized by co-injecting two aqueous solutions: one containing 1 mM Na₂CO₃ and the other 1 mM CaCl₂. By analogy, BaSO₄ nanoparticles were produced by co-injecting two aqueous solutions containing 1 mM BaCl₂ and 1 mM K₂SO₄. Iron oxide nanoparticles were synthesized by co-injecting an aqueous solution containing 1.4 mM FeCl₃ and 0.7 mM FeCl₂ and a second aqueous solution containing 0.2 M NaOH. Solutions were injected in the nebulator at 2×0.5 ml/h using volume controlled peristaltic pumps (Harvard Apparatus PHD2000 infusion syringe pumps). Fenofibrate, clotrimazole, danazol and estradiol were dissolved in ethanol at 5 mg/ml; they were injected into the nebulator in a single phase flowing at 1 ml/h. Drug particles were collected onto a single side polished silicon wafer substrate 10 cm from the nebulator outlet, while CaCO₃, BaSO₄, and iron oxide nanoparticles were collected 15 cm from the device outlet.

Sample characterization and imaging. Dried nanoparticle samples were collected on a one-side polished Si wafer for imaging and characterization using scanning electron microscopy (SEM). The samples were coated with a thin layer of Pt/Pd to minimize charging and visualized with an Ultra55 Field Emission SEM (Zeiss) operated at an extraction voltage of 5 kV using the in-lens detector. For transmission electron microscopy (TEM) analysis, samples are collected on a carbon coated 300 mesh Cu-grid (Electron Sciences). They were imaged with a JEOL2100 TEM operated at 200 kV.

XRD. X-ray diffraction was performed on spray-dried nanoparticles collected onto a one-side polished (100) Si wafer for 11 h. Measurements were acquired on a XDS2000 instrument (Scintac Inc.) using a Cu K_{α} (K alpha) source with a wavelength λ=0.154056 nm. The angle 2θ was varied between 2 and 70° at a rate of 1°/min.

DSC. Differential scanning calorimetry was performed on spray-dried nanoparticles that are collected in an aluminium based Tzero DSC pan. To obtain sufficient amounts of samples, nanoparticles were collected for 11 h before being sealed in the DSC pan with a Tzero Hermetic Lid (TA Instruments). DSC was measured on a DSC Q200 at a nitrogen flow rate of 50 ml/min (TA instruments); the temperature was increased from 40° C. to 85° C. at a rate of 1° C./min and subsequently the sample was cooled to 25° C. at the same rate. To quantify the amount of nanoparticles, thermogravimetry analysis (TGA) was performed on these samples after completing the DSC analysis. For this, the hermetically sealed pans were opened and TGA was performed on these samples by increasing the temperature from 25° C. to 400° C. at a rate of 10° C./min under a N₂ flow of 10 ml/min (Q5000, TA instrument). The absolute mass loss was measured between 100° C. and 300° C. and the DSC data was normalized accordingly.

### Example 8

Microfluidic production of attoliter-sized, droplet reaction vessels to produce nanoparticles. The small size of nanoparticles imparts properties to them that greatly differ from those of their bulk. For example, the peak plasmon absorption wavelength strongly depends on the size of gold nanoparticles. Furthermore, ferromagnetic particles become superparamagnetic if their size falls below a characteristic value. These size-dependent properties can be exploited if the nanoparticle size can be closely controlled; the extent of this control depends on the processing route. Nanoparticles typically grow from nuclei; their size depends on the growth rate of the nucleus characterized by the reaction conditions such as the solute concentration and the time nuclei can grow. Thus, monodisperse nanoparticles can be produced if nucleation and growth is separated in time; then, all nuclei grow simultaneously under identical conditions. This requires close control over the nuclei formation. However, even trace amounts of impurities or solid-liquid interfaces such as those between the solution and the reaction vessel can act as heterogeneous nucleation sites that hamper this control. This makes the production of monodisperse nanoparticles challenging and highly system specific; synthesis protocols cannot easily be generalized to the production of different types of nanoparticles.

Droplets are attractive reaction vessels that minimize the risk for uncontrolled formation of nuclei as they have no solid-liquid interfaces and the reaction volume, characterized by the drop size, is in the attoliter to picoliter range; this small volume minimizes the probability for inclusion of impurities. However, nucleation is a statistical process. Hence, if fabricated in monodisperse drops of identical composition, different drops contain varying numbers of nuclei. If these nuclei simultaneously grow until the solute contained in the drop is depleted, nanoparticles grown in a single drop have identical sizes. However, the nanoparticle size depends on the number of nuclei present in a single drop; nanoparticles produced in different drops therefore have different sizes.

The problem of the dependence of the nanoparticle size on the number of nuclei formed in a single drop can be circumvented if only one nanoparticle is formed per drop; this is possible if nanoparticles are produced in a drop, surrounded by a gas, that dries before it encounters another solid or liquid object. If multiple nuclei form in such a drop, they are pulled together by the surface tension forces of the evaporating drop resulting in a single agglomerate. Alternatively, if drops evaporate sufficiently fast, nucleation is kinetically suppressed; instead, the surface tension force of the drying drop clusters individual solute molecules together to a single, amorphous nanoparticle. In either case, the size of the resulting nanoparticle is characterized by the size of the initial drop and its primary solute concentration. Thus, if produced in monodisperse, air-born drops, nanoparticles are monodisperse. However, formation of monodisperse, air-born drops is challenging.

The exquisite flow control afforded by microfluidic technologies enables the production of highly monodisperse emulsion drops. However, microfluidic production of drops surrounded by a gas is hindered by the low viscosity of gases; this makes a controlled, on-chip break-up of drops difficult. The detailed design requirements for a successful production of these drops including the control over their size and structure remain to be shown.

This example describes design criteria to produce drops of different surface tensions inside a microfluidic device using a gas as a continuous phase, in accordance with some embodiments of the invention. This example demonstrates how the drop size can be controlled and what influence it has on the size of spray-dried nanoparticles. This technique not only allows the production of nanoparticles of controlled size and composition but also paves the way to study their nucleation and growth process on a microsecond time-scale.

The nebulator used in this example was formed from poly(dimethyl siloxane) (PDMS), and contained inlets for two types of liquids. Upon joining, the liquids entered the main channel where they intersect with the first pair of air inlets, herewith called junction 1. The angle between the liquid and the air inlet in junction 1 is called θ (theta). The 80 micrometer wide and 100 micrometer tall main channel was intersected by 1-4 additional pairs of air inlets. Located further downstream of these air inlets, there was one additional pair of air inlets; these air inlets and the main channel located further downstream that junction are 300 micrometers tall. This style of junction, herewith called three dimensional (3D) junction, fully surrounds the liquid with air, minimizing the risk that the liquid contacts the channel walls. The liquid exits the device through the nozzle outlet that is formed by slicing the main channel with a razor blade.

The microfluidic devices could be operated in the dripping regime, where drops break-up at a fixed location or in the jetting regime, where the location of drop break-up varies. At low flow rates of the dispersed and continuous phase, the devices typically were operated in the dripping regime. By contrast, if the flow rate of the dispersed or continuous phase was increased above a characteristic value, the devices were operated in the jetting regime; the jet broke into drops downstream of the junction where the two immiscible liquids form through Rayleigh-Plateau instabilities. Thus, to control the drop formation, it is important to tune the flow rates of the dispersed and continuous phase. By contrast to conventional microfluidic devices, where the continuous phase is a liquid, the nebulator operates with a gas as a continuous phase. To gain control over the drop formation in the nebulator, the velocity of the air was controlled, e.g., by tuning the pressure applied to the air inlets.

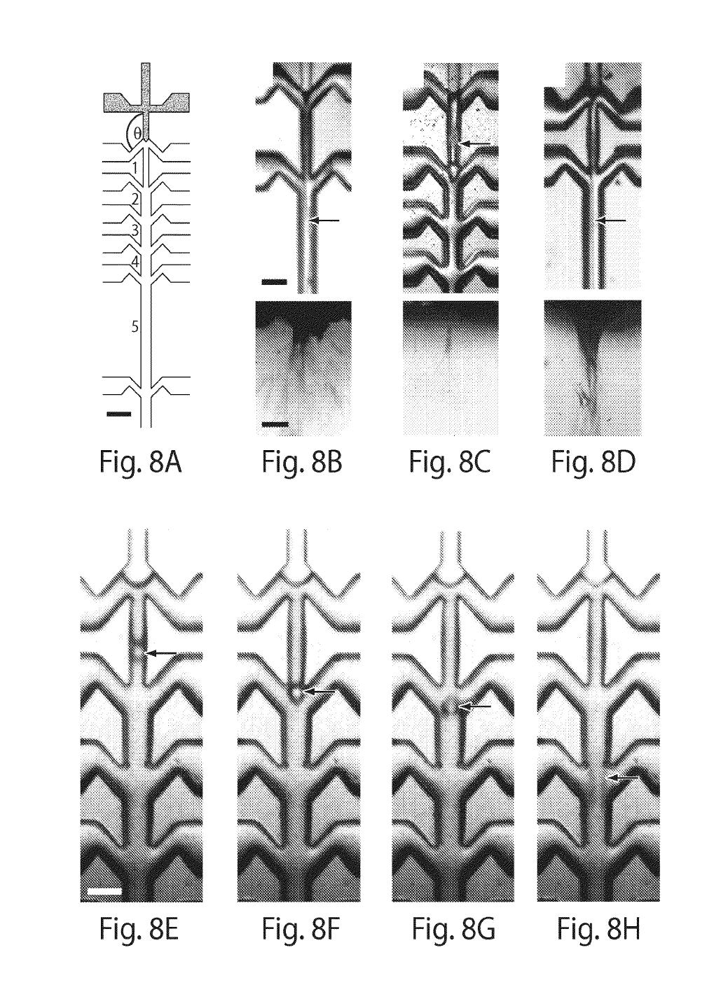

The air velocity was proportional to the pressure gradient in the main channel and inversely proportional to its resistance. The resistance was proportional to the viscosity of the continuous phase, which was three orders of magnitude lower for air than for fluids typically used in microfluidic devices making the resistance of the nebulator much lower than that of a conventional microfluidic device. Consequently, if the pressure at the air inlet is comparable to a typical value used for a conventional microfluidic experiment, the velocity of the air was three orders of magnitude higher than that of a fluid. By analogy to liquid-liquid systems, the high air velocity confined the liquid dispersed phase into a thin jet, as shown in FIG. 8A. However, by contrast to liquid-liquid systems, the high velocity of the air resulted in a slow growth of the Rayleigh-Plateau instabilities that advect downstream. In fact, the growth rate of these instabilities is too low to break the jet into drop while it is inside the 2 mm long main channel. Consequently, the jet broke into drops upon exiting the device. Thus, in some experiments, droplets were formed on-chip by operating the device in the dripping regime.

Operation of the nebulator in the dripping regime required the air velocity at the location of drop formation to be small. Thus, the pressure gradient in this region of the device should be small. This was achieved if the pressure applied to the air inlet is small. However, the propensity for liquid drops and jets to wet the channel walls, which precluded control over the drop size as drops coalesce upon touching the walls, increases with decreasing pressure. To alleviate the problem of liquids wetting the walls, the pressure applied to the air inlets was maximized. This warranted a device design that allowed a gradual pressure drop across the main channel; the pressure in junction 1 must be high but the pressure gradient low; it can be achieved if the main channel is intersected by multiple pairs of air inlets. However, even if the devices possess as many as six pairs of air inlets, the air velocity in junction 1 was still relatively high. Instead of small drops, large plugs were formed that break into smaller drops at the nozzle outlet, as shown in FIG. 8B.

Operation of the device in the dripping regime did not require the total velocity of the air to be small; instead only the velocity component parallel to the main flow direction was low. Thus, if the direction of the air inlets in junction 1 was θ=135°, this air is forced to make a U-turn to enter the main channel. Hence, the air velocity component parallel to the main channel is slowed down to close to stagnation before it is accelerated to the maximum speed reached close to the 3D junction. Indeed, devices with θ=135° and at least three pairs of 100 μm tall air inlets could be operated in the dripping regime. However, even if θ=135°, devices with only two pairs of air inlets cannot be operated in the dripping regime, as shown in FIG. 8C. This suggested that a high pressure gradient in junction 1, resulting in a high acceleration of the air in this region, pulls liquid into a thin jet in the dripping regime, irrespective of the geometry of junction 1.

The size of spray-dried nanoparticles was characterized by the initial solute concentration and the drop size. Drops produced in junction 1 of devices with θ=135° and six pairs of air inlets are approximately 80 micrometers in diameter. Thus, to use drops as reaction vessels to synthesize nm-sized particles, the droplet size was increased, and/or very low initial solute concentration was used. A low initial solute concentration resulted in a low throughput; hence, the drop size was minimized in some embodiments.

The size of the primary drops could be reduced by breaking them up into smaller secondary drops; this could be accomplished, for example, if the viscous force exceeds the surface tension force. The small dimensions of microfluidic devices, combined with the low viscosity of air, resulted in high air velocities; even if the pressure drop across the main channel is as low as 0.18 MPa, the air velocity reaches up to 740 m/s. This high velocity translated in a high viscous force; strongly deforming primary drops and breaks them into many, much smaller secondary drops, as shown in FIGS. 8D-8F.

FIG. 8A shows a schematic illustration of the nebulator containing inlets for two types of liquid (dark) and six pairs of air inlets (white), as was used in this particular example. The angle between the liquid inlet and the first pair of air inlet is called θ. The main channel is divided into sections 1-5 defined by the location of the air inlets. The air inlet pair located furthest downstream is three times as tall as the other air inlets; this type of junction, called 3D junction, fully surrounds the liquid with air. The scale bar was 200 micrometers. FIGS. 8B-8D are optical micrographs of the microfluidic nebulator (top) and its outlet (bottom). The nebulator has (FIG. 8B) θ=45° and two pairs of 100 micrometer tall air inlets, (FIG. 8C) θ=45° and five pairs of 100 micrometer tall air inlets and (FIG. 8D) θ=135° and two pairs of 100 micormeter tall air inlets. The water flow rate was 1 ml/h. The pressure applied to the air inlets was 0.28 MPa. The scale bars are 100 micrometers (top) and 200 micrometers (bottom). FIGS. 8E-8H show time lapse images of a nebulator with θ=135° and five pairs of 100 micrometer tall air inlets, operated in the dripping regime. Images are taken (FIG. 8E) 120 microseconds, (FIG. 8F) 180 microseconds, (FIG. 8G) 240 microseconds, and (FIG. 8H) 300 microseconds after the drop pinches off. The flow rate of water was 1 ml/h. The air pressure was 0.28 MPa. The scale bar is 100 micrometers.

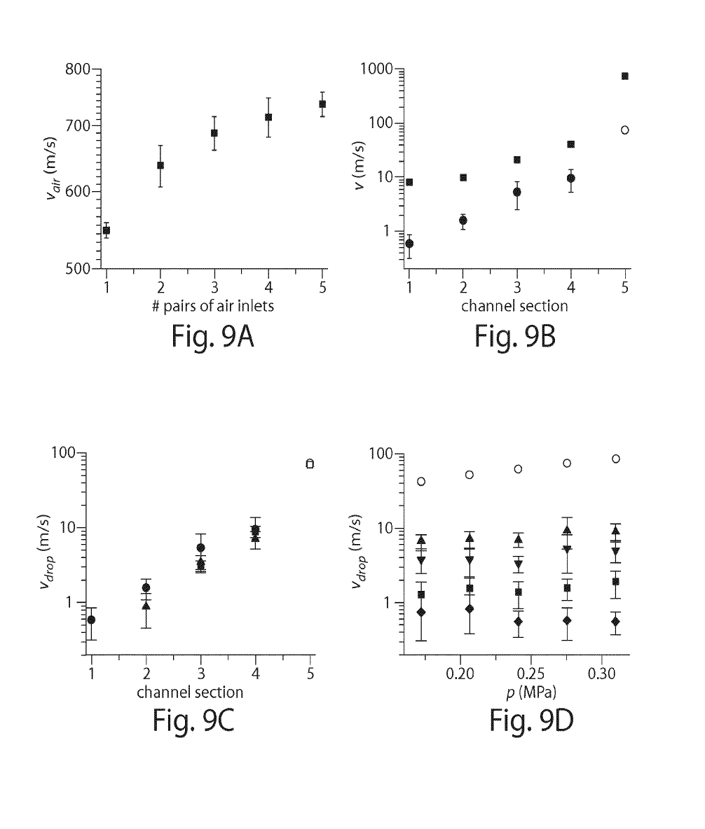

The size of these secondary drops strongly influenced the size of spray-dried nanoparticles. To gain a better understanding of the mechanism by which primary drops were broken into secondary drops, the air velocity was measured at the outlet of nebulators as a function of the number of inlets that are supplied with air. While the air velocity increased with increasing number of supplied air inlets, the difference in air velocity between adjacent channel sections decreases with increasing number or air inlets, as shown in FIG. 9A. Based on these measurements, the air velocity in the different sections of the main channel. The velocity strongly increased towards the 3D junction, as shown in FIG. 9B. To correlate the air velocity to the drop velocity, the drop velocity was measured in the different channel sections using movies acquired with a high-speed camera operated at 38000 frames per second (fps). By analogy to the air velocity, the drops are strongly accelerated in channel section 5; interestingly, it was about 10 times below the air velocity, as shown in FIG. 9B. This suggested that the final secondary drops were primarily formed in channel section 5 where the shear forces are highest.

To investigate the role of the different air inlets, the drop velocity was measured in the different channel sections of devices with 3-4 pairs of air inlets; devices with less than 3 pairs of air inlets only operate in the jetting regime. Interestingly, the strong increase in drop velocity towards the 3D junction observed for devices with 5 pairs of air inlets was also seen for those with only 3-4 pairs of air inlets. Indeed, the drop velocity was independent of the number of air inlets located further upstream the section under investigation, if the channel section located immediately upstream the 3D junction is defined as section 5, as shown in FIG. 9C. This suggested that the air velocity only depended on the number of air inlets located further downstream but not on those located further upstream the main channel section under investigation. Thus, multiple air inlets were used to ensure a low air velocity in junction 1, required for the formation of primary drops, but they only marginally influence the break-up of primary into secondary drops. However, because the air velocity in junction 1 decreased with increasing number of air inlets, the characteristic liquid flow rate above which the device jets increases with decreasing number of air inlets. It therefore may be beneficial to have multiple pairs of air inlets to ensure the nebulator operates in a stable dripping regime.

By contrast to primary drops that are strongly deformed by the viscous forces, liquid jets formed in nebulators with only two pairs of inlets, or large plugs formed in nebulators with θ=45° cannot be deformed to the same extent. This prevented or inhibited their break-up into many small secondary drops. Instead small liquid drops were sheared off the surface of these jets. However, the time required to break the entire jet into small drops by shearing off small drops from their surface exceeds the time the jet resides in the main channel section 5, where the air velocity is highest. Hence, the remaining jet that passes channel section 5 without losing its integrity is broken into big drops upon exiting the nebulator, as shown in FIGS. 8A and 8C.

If the break-up of primary into secondary drops was caused by the high shear forces, the drop size should decrease with increasing air velocity and therefore with increasing applied pressure. To differentiate between the influence of the pressure on the formation of primary and secondary drops, the speed of primary drops was measured in the different sections of the main channel as a function of pressure applied to the air inlets. Strikingly, the velocity of primary drops in channel sections 1-4 is only very weakly dependent on the air pressure. By contrast, the speed of secondary drops in channel section 5, approximated as 10% of the air speed, strongly increases with increasing pressure, as shown in FIG. 9D. This suggests that the pressure applied to the air inlets almost exclusively influences the formation of secondary drops. It was expected that the size of secondary drops to scale with the size of spray-dried nanoparticles which can be measured using scanning electron microscopy (SEM).

FIG. 9 shows flow profiles in nebulators. FIG. 9A shows that the air velocity measured at the 3D junction as a function of the number of inlet pairs supplied with air at a pressure of 0.28 MPa. FIG. 9B shows the velocity of air (squares) and the drops (circles) in the different channel sections of a nebulator with θ=135° and five pairs of 100 micrometer tall air inlets. The velocity of the drops is measured using movies acquired a with high speed camera (solid symbols) and calculated based on the air velocity (open symbols). FIG. 9C shows the velocity of water drops in the different channel sections of nebulators with θ=135° and three (squares), four (triangles), and five pairs of 100 micrometer tall air inlets (circles). The pressure at the air inlet was 0.28 MPa. Water was injected into the nebulator at 1 ml/h. FIG. 9D shows the velocity of drops in channel section 1 (diamonds), 2 (squares), 3 (triangles pointing down), 4 (triangles pointing up) and 5 (open circles) as a function of the pressure applied to the air inlets of a nebulator with θ=135° and five pairs of 100 μm tall air inlets. Water was injected at 1 ml/h.

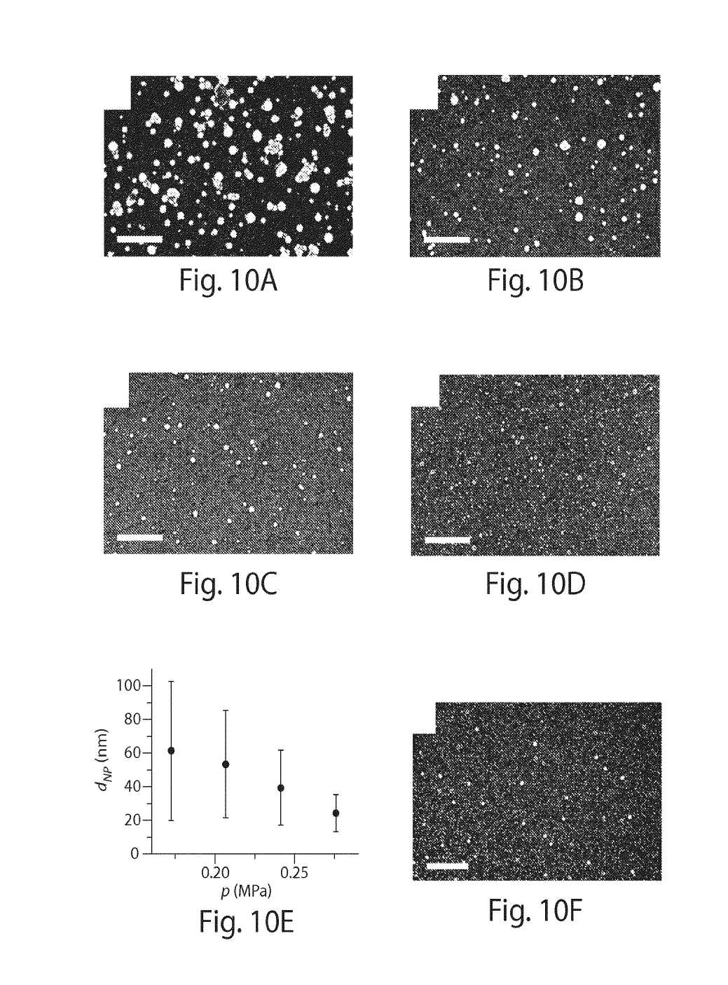

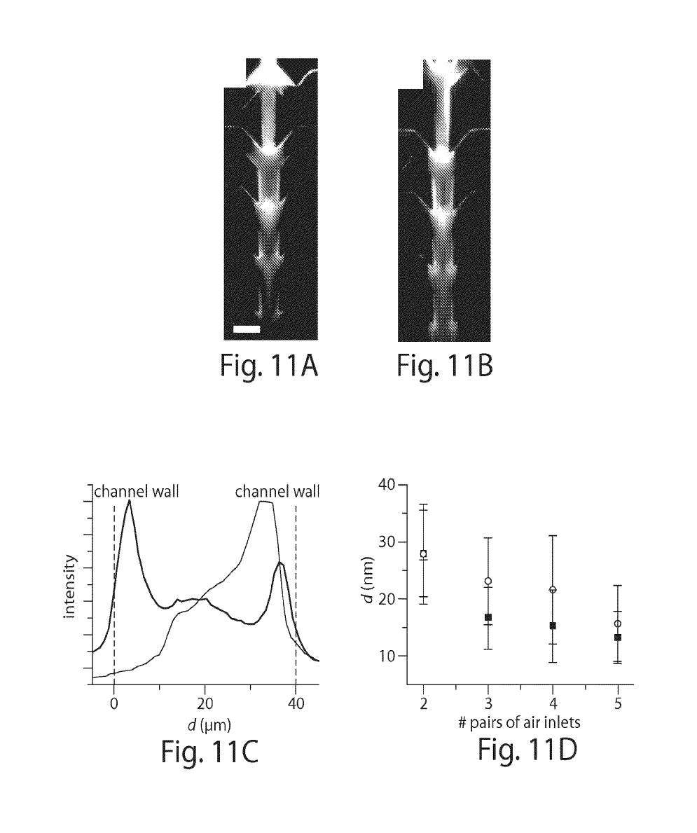

To test this expectation, CaCO₃ nanoparticles were spray dried; they were produced through a precipitation reaction by co-injecting two aqueous solutions containing CaCl₂ and Na₂CO₃ in the nebulator and collect the spray-dried particles on a Si-wafer located 20 cm apart from the nebulator nozzle. The size of CaCO₃ nanoparticles produced in devices with θ=135° and six pairs of air inlets decreased with increasing pressure applied to the air inlets, as shown in FIG. 10. Furthermore, the size distribution of particles produced using a pressure of 0.28 MPa was monomodal, suggesting a monomodal size distribution of secondary drops. By contrast, particles produced in nebulators with only two pairs of air inlets, operating in the jetting mode, display a bimodal size distribution. This suggested that, indeed, small drops are sheared off the surface of jets while they reside in channel section 5. As expected, the size of these small CaCO₃ nanoparticles was comparable to that of particles produced in devices operated in the dripping regime as the shear force, that strongly influences their size, is similar. However, by contrast to devices operated in the dripping regime, devices run in the jetting regime also produced micrometer-sized particles; these particles were likely produced in big droplets formed after the jet exits the nebulator.

If nanoparticles were produced through a precipitation reaction, nuclei can start to form upon joining the two aqueous solutions. To understand the process by which nanoparticles form, it is important to know if nuclei form in primary drops that are several tens of micrometers large or if they form in sub-100 nm sized secondary drops. To address this question, section 1 of the main channel was elongated by a factor of 3. This prolongs the time nuclei can form in primary drops before they are broken up. If the majority of CaCO₃ particles in conventional nebulators is formed in the secondary drops, a bimodal size distribution should result if channel section 1 is elongated, as this prolongs the time CaCO₃ nanoparticles can form inside primary drops where more solute molecules are available to nuclei to grow. Indeed, the size of CaCO₃ nanoparticles spray-dried with devices with an elongated channel section 1 was bimodal, supporting the suggestion that the majority of nanoparticles produced in conventional nebulators form only inside secondary drops. These experiments do not only provide insights into the mechanism by which nanoparticles form but they also demonstrate the potential of the nebulator to study the nucleation and growth mechanism of nanoparticles on a microsecond time-scale, a time window is difficult to assess with other techniques.

FIG. 10 shows spray-dried CaCO₃ nanoparticles, including scanning electron micrographs of CaCO₃ nanoparticles spray-dried in nebulators with θ=135° and five pairs of 100 micrometer tall air inlets. The pressure at the air inlets was (FIG. 10A) 0.17 MPa, (FIG. 10B) 0.21 MPa, (FIG. 10C) 0.24 MPa, and (FIG. 10D) 0.28 MPa. FIG. 10E shows the size of spray-dried CaCO₃ nanoparticles as a function of the pressure applied to the air inlets. FIG. 10F shows a scanning electron micrograph of CaCO₃ nanoparticles produced in a device with a channel section 1 that is three times larger than that of devices used to produce particles shown in FIGS. 10A-10D. The pressure at the air inlets was 0.28 MPa. The scale bars are 500 nm.

By contrast to water, the low surface tension organic solvents prevented break-up of these liquids into primary drops in junction 1, instead these liquids wet the walls. However, by analogy to the aqueous system, nanoparticles spray-dried from organic solutions, such as ethanol, have sizes below 15 nm; they therefore must be produced in drops with diameters around 80 nm. To investigate how drops of low surface tension liquids are formed in nebulators, ethanol was fluorescently labeled measure the fluorescence intensity profile was measured across the main channel in section 1. Interestingly, ethanol films homogeneously wet all four channel walls if θ=135°. By contrast, a liquid jet is pushed towards one side of the channel wall if θ=45°.

If thin films and jets are exposed to the shear force caused by the fast flowing air, small drops are sheared off the surface of these films. If the surface-to-volume ratio of the films is sufficiently high, films are broken into many small. However, if the surface-to-volume ratio of these films falls below a characteristic value, the time these films reside in areas of the nebulator, where the shear force is sufficiently high rip to drops off the liquid interface, is insufficient to completely break films into drops. Instead, the remaining films are broken up into drops downstream the 3D junction where the air velocity is lower due to the larger crosssection of the main channel or after films exit the nebulator. It was expected that nebulators that produce liquid jets with low surface-to-volume ratios would yield larger spray-dried nanoparticles than those that produce thin liquid films with high surface-to-volume ratios. To test this expectation, fenofibrate was dissolved in ethanol at an initial concentration of 5 mg/ml, sprayed using nebulators with θ=135° and θ=45°, both having 5 pairs of 100 μm tall air inlets, and the spray-dried fenofibrate particles were imaged using SEM. Remarkably, the average size and size distribution of nanoparticles produced in devices with θ=45° was consistently higher than those of particles produced in nebulators with θ=135°. This suggested that the surface-to-volume ratio of ethanol films formed in nebulators with θ=135° was sufficiently high to break the entire film before it reaches the 3D junction, in stark contrast to jets formed in devices with θ=45° where parts of the liquid is broken-up into smaller drops only after passing the 3D junction. Thus, nebulators with θ=135° were better suited to produce smallest sized nanoparticles than those with θ=45° not only if water is employed as a liquid but also if low surface tension liquids such as certain types of organic solvents are used.

If the formation of drops downstream from junction 1 is caused by the high shear force exerted on them, their size was expected to not only scale with air velocity but also with the surface tension of the liquid. To test this expectation, fenofibrate was dissolved in isopropanol, decanol and dimethyl sulfoxide (DMSO). The surface tensions of ethanol and isopropanol were similar, that of decanol is 30% higher. By contrast, the surface tension of DMSO is twice as high as that of ethanol. It was therefore expected that fenofibrate nanoparticles spray-dried from DMSO-based solutions to be significantly larger than those produced from ethanol-based solutions whereas the size of fenofibrate nanoparticles produced from the other types of solvents was not expected to differ significantly. In agreement with this expectation, the size of fenofibrate nanoparticles spray-dried from isopropanol- and decanol-based solutions was very similar to that of particles spray-dried from ethanol-based solutions. By contrast, the average size of fenofibrate nanoparticles produced from DMSO-based solution was 1.6 times that of nanoparticles produced in ethanol-based solutions. The control over the drop size was achieved by adjusting the surface tension and/or air velocity.

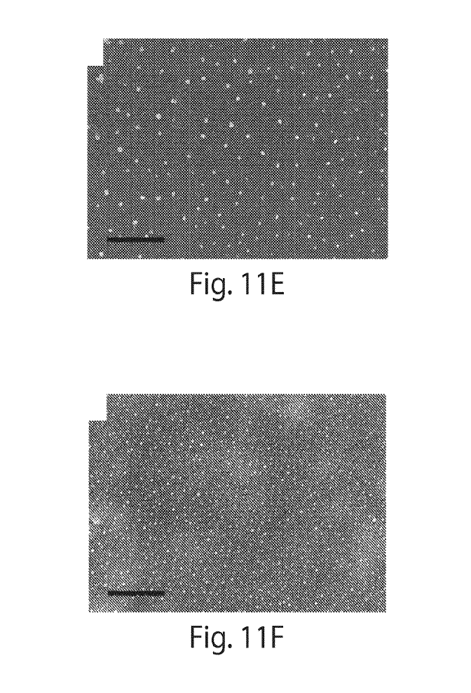

FIG. 11 shows spray drying of organic solutions. FIGS. 11A and 11B show fluorescence micrographs of a nebulator with (FIG. 11A) θ=135° and (FIG. 11B) θ=45°. Both devices had five pairs of 100 micrometer tall air inlets. The fluorescently labeled ethanol was injected at 4 ml/h. The pressure at the air inlets was 0.28 MPa. The scale bar is 100 micrometers. FIG. 11B shows the fluorescence intensity profile measured across channel section 1 of the nebulator with θ=135° (left peak) and θ=45° (right peak). FIG. 11C shows that the size of fenofibrate nanoparticles produced in nebulators with θ=135° (squares) and θ=45° (circles) and different numbers of air inlets. The pressure at the air inlets was 0.28 MPa. The ethanol flow rate was 1 ml/h. FIGS. 11E and 11F are scanning electron micrographs of fenofibrate nanoparticles produced in devices with (FIG. 11E) θ=135° and (FIG. 11F) θ=45°. The scale bars are 500 nm.

In conclusion, design criteria for microfluidic devices that enable the formation of air-born drops with diameters below 100 nm were presented. Smallest-sized drops are formed inside the nebulator due to the high shear forces that act on liquids and broke it into drops. These high shear forces are a result of the small dimensions of microfluidic channels through which the air flows; even if the pressure applied to the air inlets does not exceed 0.3 MPa, the air reached supersonic speeds. It was demonstrated the use of these attoliter-sized drops as reaction vessels to fabricate monodisperse nanoparticles whose size is characterized by the initial solute concentration, the drop size, and the nebulator design. However, the nebulator does not only allow control over the size of spray-dried nanoparticles but also over their structure; this control was achieved by adjusting the time nanoparticles can grow inside the liquid before it is broken into sub-micrometer sized drops. The nebulator used in this particular non-limiting example was a powerful tool not only to produce nanoparticles, but also to gain scientific insights into the mechanism by which they form.

### Example 9

This example demonstrates a method to produce amorphous nanoparticles that are contained in an excipient matrix. This is achieved by spraying amorphous nanoparticles onto a layer of excipients thereby facilitating their handling. This example also shows that excipients that are co-spray dried with drugs can act as heterogeneous nucleation sites, thereby promoting the formation of crystalline nuclei. These small crystals can often not be detected with XRD, making the materials initially XRD-amorphous. However, they compromise the long-term stability of amorphous nanoparticles; a significant fraction of amorphous drug nanoparticles containing these nuclei crystallizes within two weeks; this is significantly faster than amorphous nanoparticles produced in the absence of excipients. If pure drugs are spray dried onto a matrix of excipients, they retain the excellent long-term stability of amorphous nanoparticles produced in the absence of excipients but the presence of excipients facilitates their handling.

The nebulator used in this example was a poly(dimethyl siloxane) (PDMS)-based microfluidic device containing inlets for two types of liquids. Upon merging, the liquids intersect the first pair of air inlets; the angle between the liquid and air inlet is 135°. Four additional pairs of air inlets intersect the main channel further downstream at an angle of 45°. These air inlets are 80 micrometers wide and 100 micrometers tall. There is one additional pair of air inlet located furthest downstream; these inlets and the main channel located further downstream that junction are 300 micrometers tall, making this junction three dimensional (3D). The nebulator outlet is formed by slicing the main channel with a razor blade.

The nebulator allowed production of amorphous drug nanoparticles with sizes below 20 nm in this example. However, to control the dissolution kinetics of drug nanoparticles, their size was controlled. The size was expected to scale with the drop size, by analogy to that of inorganic nanoparticles. To test this, 5 mg/ml fenofibrate was dissolved in ethanol and this solution was injected into the nebulator at 1 ml/h. The inlet for the second type of liquid was clogged and pressures between 0.17 and 0.28 MPa were applied to the air inlets. Spray dried nanoparticels were collected on a one-side polished Si-wafer located 10 cm apart from the nozzle outlet and measure their size using scanning electron microscopy (SEM). In agreement with this expectation, the size of spray dried fenofibrate nanoparticles decreased with increasing pressure applied to the air inlets. This suggests that the drop size at the nebulator outlet decreased with increasing pressure, by analogy to water drops produced in the nebulator.

The drop size not only influenced the size of spray dried nanoparticles but also their structure; crystalline nuclei can form when the solute concentration exceeds its saturation concentration and they stop forming when the drop is completely dried. Therefore, the time nuclei can form depends on the time it takes to dry a drop if the initial solute concentration was kept constant; it increased with increasing drop and therefore nanoparticle size. Thus, large particles, formed in big drops, were more likely to contain crystalline nuclei than small ones. This example studied amorphous particles; thus, certain experiments focused on 15 nm sized nanoparticles produced in 85 nm sized ethanol drops. These droplets were formed in the nebulator by applying 0.28 MPa to the air inlets. The time crystalline nuclei can form in these drops is 1.6 microseconds, this is too short for them to form; thus, nanoparticles produced under these conditions were amorphous.

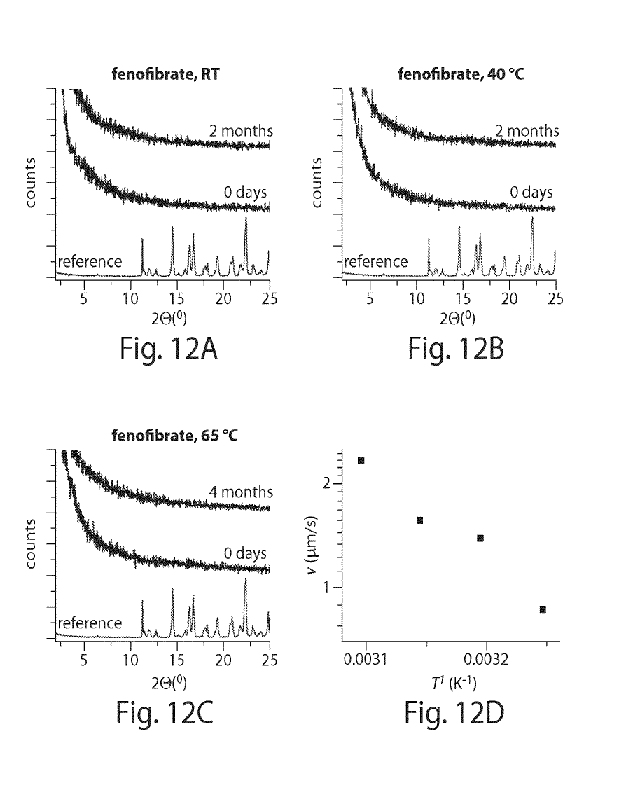

Fenofibrate has a glass transition temperature T_{g} of −20° C.; it is an undercooled liquid at room temperature. It was expected to crystallize over time if stored at room temperature even if it is initially fully amorphous. Surprisingly, it remained XRD-amorphous even if stored at 65° C. for more than 4 weeks, as is shown in FIG. 12. To elucidate the reason for this high stability of the amorphous phase, crystal growth was decoupled from the formation of crystalline nuclei by seeding an undercooled liquid with a fenofibrate crystal and acquiring a time lapse of confocal images for different temperatures for growing crystals. Notably, the undercooled liquid rapidly crystallized if seeded with a fenofibrate crystal at 35° C.

FIG. 12 shows stability of amorphous fenofibrate. FIGS. 12A-C show X-ray diffraction (XRD) spectra of fenofibrate directly after spray drying (middle) and after incubating the sample at (FIG. 12A) 20° C., (FIG. 12B) 40° C. and (FIG. 12C) 65° C. for 1-2 months (top). The reference spectrum of crystalline fenofibrate is shown at the bottom. FIG. 12 D shows the maximum growth rate of the crystal as a function of the temperature.

It was expected that temperature-dependent growth rate of the crystals would display an Arrhenius-like behavior. To test this expectation, the maximum speed crystals grown was measured at as a function of the temperature using the time-lapse confocal micrographs. In good agreement with this expectation, the growth rate displays an Arrhenius-like behavior. It was more than three times higher at 50° C. than it is at 35° C. Extrapolating the crystal growth rate to room temperature, it was found that the crystal growth rate at 20° C. was 1.6 mm/h. This suggested that nucleation was the rate-limiting step in the crystallization of amorphous fenofibrate, by analogy to most organic compounds and demonstrated the importance to completely suppress formation of crystalline nuclei to achieve long-term stability of amorphous drugs.

To experimentally test the difference in solubility between the amorphous and crystalline phase, amorphous fenofibrate was spray dried onto a microscopy slide and a fenofibrate crystal was grown next to it by slowly evaporating an fenofibrate containing ethanol solution. The behavior of the amorphous and crystal phase at 35° C. if contacted with a drop of water was simultaneously imaged using confocal microscopy. Indeed, the amorphous phase dissolved significantly faster and in higher quantities than the crystal. This is in agreement with computations that predict a 15 times higher solubility of the amorphous phase compared to its crystal.

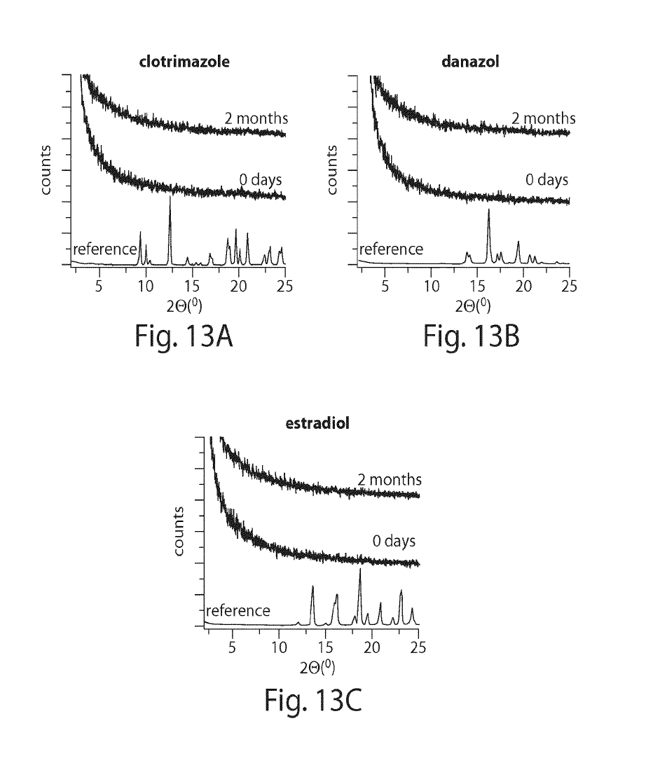

To test if the nebulator also allowed production of amorphous nanoparticles from materials with a T_{g} below room temperature, which would be a glass at room temperature, clotrimazole, estradiol, and danazol, all poorly water soluble drugs with T_{g}s above 20° C., were spry dried. The size of the spray dried nanoparticles was determined by the initial solute concentration and the initial drop size. To ensure similar sizes of the spray dried nanoparticles, the initial solute concentration was fixed to 5 mg/ml. Remarkably, all these drugs were amorphous and did not crystallize for at least 2 months if stored at room temperature, as is shown in FIG. 13. Thus, the nebulator allowed formulation of poorly water soluble drugs as amorphous nanoparticles. For substances that had a T_{g} below room temperature, they were an undercooled liquid at room temperature, and those with T_{g} above room temperature, they were a glass at room temperature, without requiring the addition of excipients.

FIG. 13 shows spray drying drugs with a T_{g} above room temperature. These are XRD spectra of the crystalline drug (bottom) spray dried drug directly after the sample is prepared (middle) and after storing the sample under ambient conditions at 20° C. for 2 months (top). FIG. 13A is clotrimazole, FIG. 13B is danazol, and FIG. 13C is estradiol.

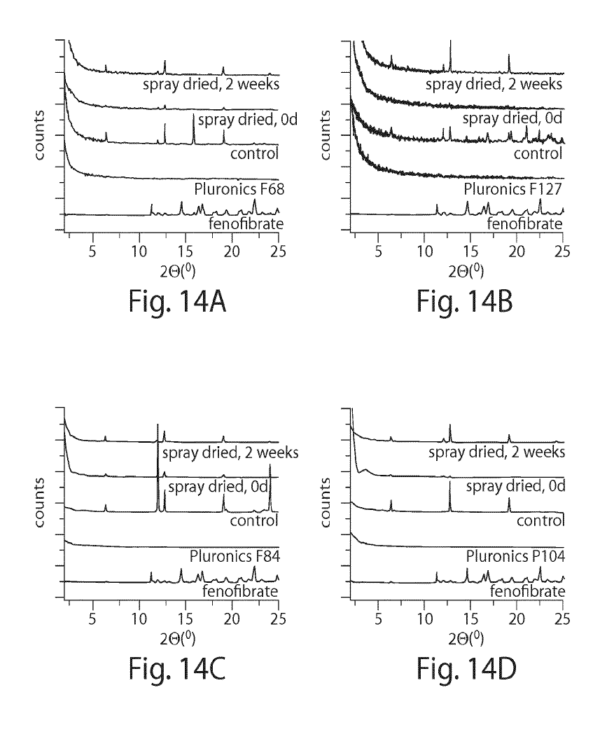

To ease the handling drugs, fenofibrate was co-spray dried with different types of excipients. 5 mg/ml fenofibrate and 5 mg/ml Pluronics was dissolved in ethanol and spray drie using the same conditions than used to spray dry pure fenofibrate. Four different types of Pluronics were compared: F68 and F127 are solids at room temperature and Pluronics P84 and P104 are pastes at room temperature. While fenofibrate nanoparticles were embedded in Pluronics F68 and F127, individual nanoparticles were easily discernible if co-spray dried with Pluronics P84 or Pluronics P104. Surprisingly, fenofibrate nanoparticles spray dried in the presence of any of these types of excipients contain crystalline regions, as indicated by X-ray diffraction (XRD) spectra. Importantly, a significant fraction of fenofibrate, that initially is XRD-amorphous, crystallizes within two weeks if stored under ambient conditions. See FIG. 14. This is in stark contrast to fenofibrate nanoparticles spray dried in the absence of excipients; they are stable for at least two months if stored under identical conditions.

FIG. 14 shows co-spray drying fenofibrate with Pluronics excipients. Fenofibrate is co-spray dried with (FIG. 14A) Pluronics F68, (FIG. 14B) Pluronics F127, (FIG. 14C) Pluronics P84, and (FIG. 14D) Pluronics P104. From bottom to top: XRD spectra of crystalline fenofibrate, spray dried Pluronics, fenofibrate mixed with equal weights of Pluronics dissolved in ethanol and slowly dried in air, fenofibrate, mixed with equal weights of Pluronics dissolved in ethanol and spray dried directly after the sample is prepared, and after storing the sample for 2 weeks under ambient conditions.

To test if the higher propensity of fenofibrate to crystallize if spray dried in the presence of Pluronics is related to the fact, that it is an undercooled liquid at room temperature if amorphous, danazol, with a T_{g} above room temperature, was co-spray dried with the identical excipients. By analogy to fenofibrate, danazol particles spray dried in the presence of these excipients also contain a significant fraction of crystalline regions; this fraction markedly increases after storing the sample for two weeks under ambient conditions, as is shown in FIG. 15.

FIG. 15 shows co-spray drying danazol with Pluronic excipients. Danazol is co-spray dried with (FIG. 14A) Pluronics F68, (FIG. 14B) Pluronics F127, (FIG. 14C) Pluronics P84, and (FIG. 14D) Pluronics P104. Bottom to top: XRD spectra of crystalline danazol, spray dried Pluronics, danazol mixed with equal weights of Pluronics dissolved in ethanol and slowly dried in air, danazol mixed with equal weights of Pluronics dissolved in ethanol and spray dried directly after the sample is prepared, and after storing the sample for 2 weeks under ambient conditions.

These results suggested that excipients act as heterogeneous nucleation sites, thereby promoting the formation of crystalline nuclei during the spray dry process irrespective of the T_{g} of the drug. Importantly, even if drugs prepared in the presence of excipients are XRD-amorphous directly after their preparation, such as fenofibrate that is co-spray dried with Pluronics P104, it crystallizes within two weeks. The much lower stability of these nanoparticles indicated that the formation of crystalline nuclei was not completely suppressed during the spray dry process if any of the tested excipients are co-spray dried with the drug; these crystalline nuclei subsequently grew by consuming the amorphous drug. Because nucleation as the rate limiting step in the crystallization of these drugs, the stability of amorphous drugs containing crystalline nuclei was much lower than that of fully amorphous ones.

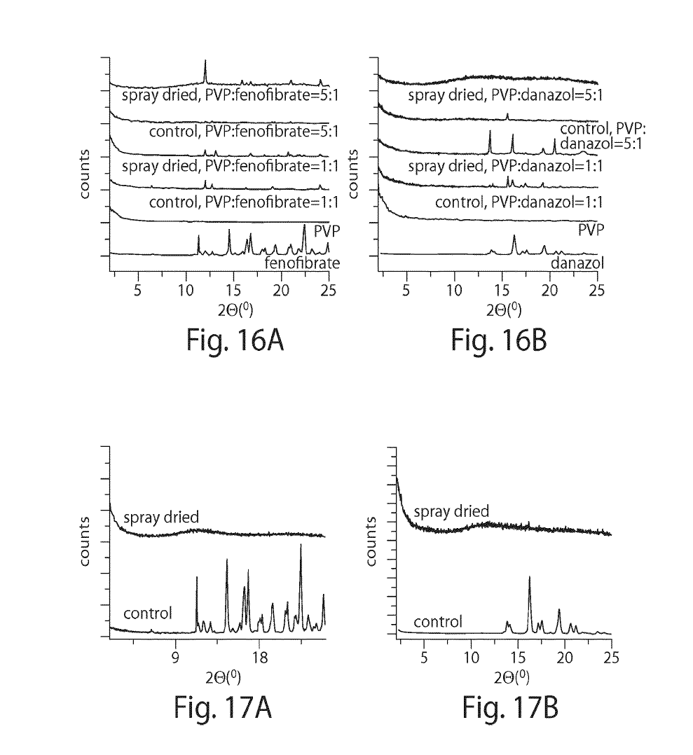

To test if the inferior stability of amorphous nanoparticles spray dried in the presence of Pluronics is related to the chemical composition of the excipient, fenofibrate and danazol were co-spray dried with poly(vinyl pyrrolidone) (PVP), an excipient often used to suppress the formation of crystalline nuclei in drugs. By analogy to Pluronics, drugs spray dried from solutions containing equal weights of drugs and excipients were crystalline. By contrast, increasing the weight fraction of PVP five-fold results in XRD-amorphous danazol nanoparticles, in agreement to danazol nanoparticles formulated using the microfluidic spray drier. However, fenofibrate particles co-spray dried with PVP were still crystalline, even if the concentration of PVP is five times higher than that of the drug. See FIG. 16. This demonstrated the main disadvantage of using excipients to suppress the formation of crystalline nuclei: they were highly system-specific and appropriate excipients must be identified for each system separately. These findings further suggest that all the tested excipients promote formation of crystalline nuclei, thereby decreasing the stability of amorphous nanoparticles.

FIG. 16 shows co-spray drying drugs with poly(vinyl pyrrolidone) (PVP) with XRD spectra of (FIG. 16A) fenofibrate and (FIG. 16B) danazol. The spectra are (bottom to top) crystalline drug, spray dried PVP, drug mixed with PVP at a weight ratio of 1:1 dissolved in ethanol and slowly dried in air, and the same solution spray dried, drug mixed with PVP at a weight ratio of 1:5 dissolved in ethanol and slowly dried in air, and the same solution spray dried.

To maintain the stability of spray dried amorphous nanoparticles and facilitate their handling by embedding them in an excipient matrix, PVP was deposited on a Si-wafer and subsequently drugs were spray dried into the PVP matrix. It was found that both fenofibrate and danazol nanoparticles were XRD-amorphous if spray dried on a PVP matrix. See FIG. 17. However, because excipients were not required to make the drug amorphous but rather to ease their handling, this procedure is generally applicable to the formulation of many different types of drugs, by contrast to the solid dispersions where the appropriate excipients must be selected for each system individually. Thus, the nebulator used in this example allowed the formulation of amorphous drug nanoparticles that optionally could be sprayed into an excipient matrix to facilitate their handling. However, the nanoparticles were amorphous because the formation of crystalline nuclei was kinetically suppressed and did not appear to be due to the presence of excipients. Thus, the amorphous structure does not depend on the choice of excipients but on the initial solute concentration and initial drop size, parameters that can easily be tuned during operation.

(FIG. 17A) Fenofibrate and (FIG. 17B) danazol is spray dried onto a PVP matrix. XRD spectra of the crystalline drug (bottom) and drugs spray dried onto a PVP matrix (top) are shown. The PVP matrix is formed by depositing an ethanol solution containing 25 mg/ml PVP on a polished Si-wafer and slowly evaporating the ethanol in air.

This example illustrates one method to produce amorphous drug nanoparticles that are embedded in an excipient matrix; it was achieved through the use of a microfluidic nebulator that produces amorphous drug nanoparticles with sizes below 20 nm. They were sprayed into an excipient matrix to ease their handling. By contrast to solid dispersions, the amorphous structure of the drug did not rely on interactions with excipients, but on the fast evaporation of drops that kinetically suppresses the formation of crystalline nuclei during the spray dry process. Thus, amorphous drug nanoparticles could be embedded into different types of excipients without compromising their stability. This method is generally applicable to the formulation of many different types of amorphous drug nanoparticles that can be embedded into a variety of excipients; this makes the identification of an appropriate excipient for each drug superfluous and therefore significantly facilitates the formulation of amorphous drug nanoparticles that are embedded in an excipient matrix.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method, comprising:
providing a fluidic droplet having an average diameter of less than about 100 nm, wherein the droplet initially comprises less than about 10 wt % of a species contained within a fluid, and wherein the species has a solubility of at least about 0.1 g/L in the fluid; and
drying the fluidic droplet within a microfluidic channel to remove at least about 30 wt % of the fluid from the droplet to produce a substantially amorphous particle comprising the species.

2. The method of claim 1, wherein the particle is substantially amorphous as determined using differential scanning calorimetry.

3. The method of claim 1, wherein the particle is substantially amorphous as determined using transmission electron microscopy.

4. The method of claim 1, wherein the particle is substantially amorphous as determined using x-ray diffraction.

5. The method of claim 1, wherein the species is at least partially dissolved within the fluid.

6. The method of claim 1, wherein the species comprises a dissolved ionic salt.

7. (canceled)

8. The method of claim 1, wherein the droplets have an average diameter of less than about 100 nm.

9-13. (canceled)

14. The method of claim 1, wherein the species has a solubility of at least about 1 g/L in the fluid.

15-16. (canceled)

17. The method of claim 1, comprising drying the fluidic droplet to remove at least about 75 wt % of the fluid from the droplet.

18-19. (canceled)

20. The method of claim 1, wherein at least about 50 wt % of the amorphous particle comprises the species.

21-22. (canceled)

23. A composition, comprising:
a plurality of particles that are substantially amorphous, wherein at least about 90% of the particles comprise at least about 75 wt % of a metal.

24. (canceled)

25. The composition of claim 23, wherein the particles have an average diameter of no more than about 50 nm.

26-29. (canceled)

30. The composition of claim 1, wherein at least about 90% of the particles comprise at least about 90 wt % of a pure metal.

31. (canceled)

32. The composition of any claim 23, wherein the pure metal is selected from the group consisting of beryllium, magnesium, zinc, aluminum, gallium, indium, iron, cobalt, copper, titanium, gold, silver, and nickel.

33. A method of evaporating a liquid, comprising:
passing a liquid droplet comprising a metal through a microfluidic channel such that at least about 20 vol % of the liquid evaporates from the droplet while the droplet is contained within the microfluidic channel to produce a substantially amorphous particle comprising at least 75 wt % of the metal.

34. (canceled)

35. The method of any claim 33, wherein at least about 50 vol % of the liquid evaporates while the droplet is contained within the microfluidic channel.

36-37. (canceled)

38. The method of any claim 33, wherein the liquid is miscible in water.

39. (canceled)

40. The method of any claim 33, wherein the liquid within the microfluidic channel is surrounded by a gas.

41-43. (canceled)

44. The method of claim 33, wherein the liquid droplet solidifies into the particle prior to exiting the microfluidic channel.

45. The method of claim 33, wherein the liquid droplet solidifies into the particle after exiting the microfluidic channel.

46-102. (canceled)
